(19) United States
(12) Patent Application Publication
(10) Pub. No.: US 2024/0115726 A1
(43) Pub. Date: Apr. 11, 2024

(54) HYDRAZONE-BASED SAPONIN DERIVATIVES

(71) Applicant: SAPREME TECHNOLOGIES B.V., Bilthoven (NL)

(72) Inventors: Ruben POSTEL, Bilthoven (NL); Guy HERMANS, Bilthoven (NL)

(21) Appl. No.: 18/012,791

(22) PCT Filed: Jun. 22, 2021
(86) PCT No.: PCT/NL2021/050392

§ 371 Date: Dec. 23, 2022

(30) Foreign Application Priority Data
Jun. 24, 2020 (NL) 2025904
Jan. 26, 2021 (NL) 2027439

(51) Int. Cl. A61K 47/68; A61K 47/54

(52) U.S. Cl. CPC A61K 47/6851; A61K 47/542; A61K 47/545; A61K 47/549; A61K 47/6889

(57) ABSTRACT

The invention relates to a saponin derivative The invention also relates to a first pharmaceutical composition comprising the saponin derivative of the invention. In addition, the invention relates to a pharmaceutical combination comprising the first pharmaceutical composition of the invention and a second pharmaceutical composition comprising for example an ADC or AOC. The invention also relates to the first pharmaceutical composition or the pharmaceutical combination of the invention, for use as a medicament, or use in the treatment or prophylaxis of a cancer or an auto-immune disease. Furthermore, the invention relates to an in vitro or ex vivo method for transferring a molecule from outside a cell to inside said cell, comprising contacting said cell with the molecule and with a saponin derivative of the invention. The invention also relates to a saponin conjugate comprising a cell-surface molecule binding-molecule, capable of binding to a target cell, covalently bound to the saponin. The invention also relates to a pharmaceutical combination comprising a pharmaceutical composition comprising the saponin conjugate of the invention and a second pharmaceutical composition comprising an active pharmaceutical ingredient, or to a pharmaceutical composition comprising the saponin conjugate of the invention and an active pharmaceutical ingredient. In addition, the invention relates to said pharmaceutical combination of the invention or said pharmaceutical composition of the invention, for use as a medicament, or for use in the treatment or the prophylaxis of a cancer or an auto-immune disease.

### CROSS-REFERENCE TO RELATED APPLICATIONS

This application is the National Stage of International Application No. PCT/NL2021/050392, filed on Jun. 22, 2021, which claims the benefit of and priority to Netherlands Patent Application No. 2025904, filed on Jun. 24, 2020, and Netherlands Patent Application No. 2027439, filed on Jan. 26, 2021, the contents of which are incorporated by reference in their entirety.

### TECHNOLOGICAL FIELD

The invention relates to a saponin derivative based on a saponin comprising a triterpene aglycone and a saccharide chain linked to the aglycone core structure, wherein the saponin derivative comprises an aglycone core structure comprising an aldehyde functional group which has been transformed to a hydrazone functional group. The invention also relates to a first pharmaceutical composition comprising the saponin derivative of the invention. In addition, the invention relates to a first pharmaceutical combination comprising the first pharmaceutical composition of the invention and a second pharmaceutical composition comprising e.g. an ADC, AOC. The invention also relates to the first pharmaceutical composition or the first pharmaceutical combination of the invention, for use as a medicament, or use in the treatment or prophylaxis of a cancer or an auto-immune disease. Furthermore, the invention relates to an in vitro or ex vivo method for transferring a molecule from outside a cell to inside said cell, comprising contacting said cell with the molecule and with a saponin derivative of the invention. The invention also relates to a saponin conjugate comprising a cell-surface molecule binding-molecule, capable of binding to a cell, and covalently bound to the saponin. The invention also relates to a second pharmaceutical combination comprising a pharmaceutical composition comprising the saponin conjugate of the invention and a pharmaceutical composition comprising an active pharmaceutical ingredient, such as an ADC or AOC, or to a pharmaceutical composition comprising the saponin conjugate of the invention and an active pharmaceutical ingredient. In addition, the invention relates to such pharmaceutical combination of the invention or such pharmaceutical composition of the invention, for use as a medicament, or for use in the treatment or the prophylaxis of a cancer or an auto-immune disease.

### BACKGROUND OF THE INVENTION

Targeted tumor therapy is a cancer treatment that uses drugs to target specific genes and proteins that are involved in the growth and survival of cancer cells. Immunotoxins, which are targeted toxins that contain an antibody as targeting moiety, are very promising because they combine the specificity of an antibody against tumor-specific antigens, which enables them to channel the toxin to the aimed point of action, and can introduce additionally cell killing mechanisms such as antibody-dependent cell-mediated cytotoxicity and complement-dependent cytotoxicity. To exhibit its effect, the toxin needs to be released into the cytosol after internalization. A major drawback is that the targeting moiety which bears the payload is often not fully internalized, directly recycled to the surface after internalization, or degraded in lysosomes, therewith hampering the sufficient delivery of the payload into the cell cytosol. To ensure a toxic payload concentration for tumor cells and to overcome insufficient cytosolic entry, high serum levels of the targeted toxin are required often resulting in severe side effects, in particular including immunogenicity and vascular leak syndrome. Thus, a sufficiently wide therapeutic window remains a concern when treating cancer patients with antibody-drug conjugates (ADCs).

To cope with the drawback of insufficient cytosolic entry, several strategies were developed relating to for example the redirection of toxins to endogenous cellular membrane transport complexes of the biosynthetic pathway, disruption of endosomes, attenuation of the membrane integrity of endosomal membranes, or use of cell penetrating peptides.

For example, glycosylated triterpenes such as saponins were found to act as endosomal escape enhancers for targeted toxins, such as ribosome-inactivating proteins (RIPs), in tumor therapy. Structural-activity relationship analysis of saponins revealed that the presence of inter alia an aldehyde at the C-4 position appears to be beneficial for the ability of saponins to enhance the cytotoxicity of RIPs (see Formula (1) with A¹=H or OH and A²=a polysaccharide moiety).

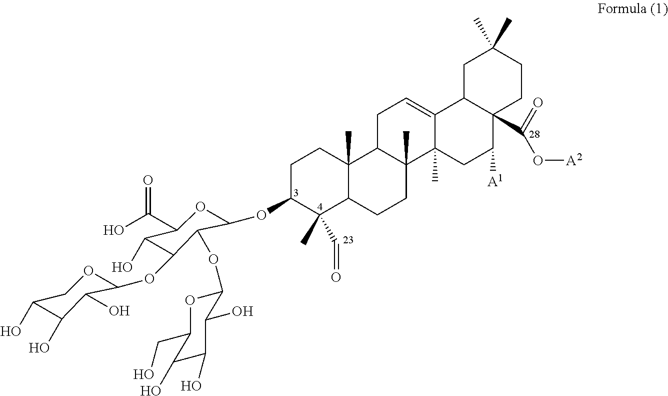

Especially, saponin SO1861 (Formula (2), sometimes also referred to as SPT001 or SPT1), a triterpenoid saponin, was identified as a potent molecule in order to enhance the endosomal escape of tumor-cell targeted toxins. A dual effect for the enhancer mechanism is postulated: first, a direct increase of the endosomal escape resulting in caspase-dependent apoptosis that is, second, combined with lysosomal-mediated cell death pathways, which are triggered after the release of cathepsins and other hydrolytic enzymes following destruction of lysosomal membranes.

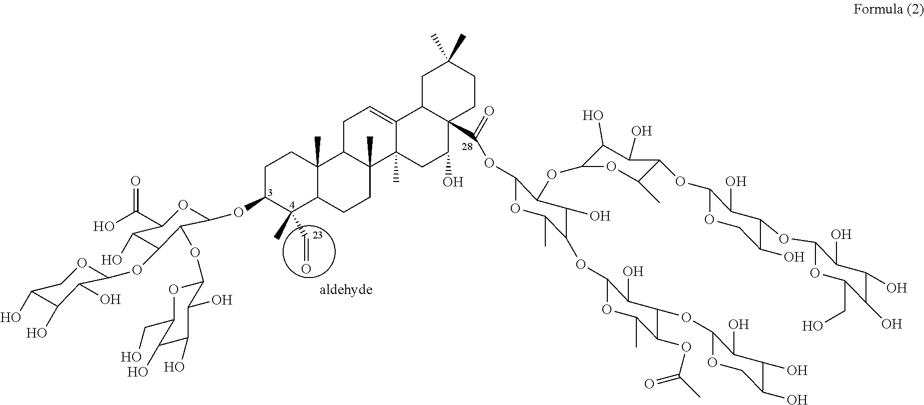

The application of saponins as endosomal escape enhancers is based on the recognition that these saponins have the ability to rupture erythrocyte membranes. However, at the very same time, cell rupturing activity of saponins contribute to (the risk for) side effects when a subject is treated with such saponins, therewith influencing optimal therapeutic windows in view of limiting therapeutic index. Indeed, toxicity of such saponins, extracellularly and/or intracellularly, when administered to a patient in need of anti-tumor therapy, is of concern when for example the optimal dosing regimen and route and frequency of administration are considered.

All characteristics of the chemical composition of the saponins themselves, including the structure of the triterpene backbone, a pentacyclic C30 terpene skeleton (also known as sapogenin or aglycone), number and length of saccharide side chains as well as type and linkage variants of the sugar residues linked to the backbone, contribute to the hemolytic potential and/or cytotoxicity of such saponins.

The saponins are per se not target-specific when the endosome and the cytosol of cells are considered, and saponins expectedly and most often distribute in a (human) subject with other kinetics than the targeted toxins, even when the same route of administration would be considered for a combination of a saponin and e.g. an ADC. Thus, after application to a patient in need thereof of a therapeutic combination comprising e.g. an ADC and a saponin, the saponin molecules can be found in any organ connoting that specificity is only mediated by the targeted toxin. Distribution of saponins in the whole body (systemic distribution) requires higher concentrations for a successful treatment when compared to specific accumulation in target cells as is achieved for the ADC. Hence, the toxicity of the modified saponins needs to be low enough for a successful application in view of the systemic application of saponins in the body, in order to achieve a suitable therapeutic window.

Therefore, there is a still a need to improve the therapeutic index when co-administration of a saponin together with e.g. an ADC is considered: need for better controlling (or better: lower) the cytotoxicity of saponins while at the same time maintaining sufficient efficacy when potentiation of the cytotoxic effect of an ADC is considered.

ADCs are mainly composed of an antibody, a cytotoxic moiety such as a payload, and a linker. Several novel strategies have been proposed and carried out in the design and development of new ADCs to overcome the existing problems, targeting each of the components of ADCs. For example, by identification and validation of adequate antigenic targets for the antibody component, by selecting antigens which have high expression levels in tumor and little or no expression in normal tissues, antigens which are present on the cell surface to be accessible to the circulating ADCs, and antigens which allows internalizing of ADCs into the cell after binding; and alternative mechanisms of activity; design and optimize linkers which enhance the solubility and the drug-to-antibody ratio (DAR) of ADCs and overcome resistance induced by proteins that can transport the chemotherapeutic agent out of the cells; enhance the DAR ratio by inclusion of more payloads, select and optimize antibodies to improve antibody homogeneity and developability. In addition to the technological development of ADCs, new clinical and translational strategies are also being deployed to maximize the therapeutic index, such as, change dosing schedules through fractionated dosing; perform bio-distribution studies; include biomarkers to optimize patient selection, to capture response signals early and monitor the duration and depth of response, and to inform combination studies.

An example of ADCs with clinical potential are those ADCs such as brentuximab vedotin, inotuzumab ozogamicin, moxetumomab pasudotox, and polatuzumab vedotin, which are evaluated as a treatment option for lymphoid malignancies and multiple myeloma. Polatuzumab vedotin, binding to CD79b on (malignant) B-cells, and pinatuzumab vedotin, binding to CD22, are tested in clinical trials wherein the ADCs each were combined with co-administered rituximab, a monoclonal antibody binding to CD20 and not provided with a payload [B. Yu and D. Liu, Antibody-drug conjugates in clinical trials for lymphoid malignancies and multiple myeloma; Journal of Hematology & Oncology (2019) 12:94]. Combinations of monoclonal antibodies such as these examples are yet a further approach and attempt to arrive at the ‘magic bullet’ which combines many or even all of the aforementioned desired characteristics of ADCs.

Meanwhile in the past few decades, nucleic acid-based therapeutics are under development. Therapeutic nucleic acids can be based on deoxyribonucleic acid (DNA) or ribonucleic acid (RNA), Anti-sense oligonucleotides (ASOs, AONs), and short interfering RNAs (siRNAs), MicroRNAs, and DNA and RNA aptamers, for approaches such as gene therapy, RNA interference (RNAi). Many of them share the same fundamental basis of action by inhibition of either DNA or RNA expression, thereby preventing expression of disease-related abnormal proteins. The largest number of clinical trials is being carried out in the field of gene therapy, with almost 2600 ongoing or completed clinical trials worldwide but with only about 4% entering phase 3. This is followed by clinical trials with ASOs. Similarly to ADCs, despite the large number of techniques being explored, therapeutic nucleic acids share two major issues during clinical development: delivery into cells and off-target effects. For instance, ASOs such as peptide nucleic acid (PNA), phosphoramidate morpholino oligomer (PMO), locked nucleic acid (LNA) and bridged nucleic acid (BNA), are being investigated as an attractive strategy to inhibit specifically target genes and especially those genes that are difficult to target with small molecules inhibitors or neutralizing antibodies. Currently, the efficacy of different ASOs is being studied in many neurodegenerative diseases such as Huntington's disease, Parkinson's disease, Alzheimer's disease, and amyotrophic lateral sclerosis and also in several cancer stages. The application of ASOs as potential therapeutic agents requires safe and effective methods for their delivery to the cytoplasm and/or nucleus of the target cells and tissues. Although the clinical relevance of ASOs has been demonstrated, inefficient cellular uptake, both in vitro and in vivo, limit the efficacy of ASOs and has been a barrier to therapeutic development. Cellular uptake can be <2% of the dose resulting in too low ASO concentration at the active site for an effective and sustained outcome. This consequently requires an increase of the administered dose which induces off-target effects. Most common side-effects are activation of the complement cascade, the inhibition of the clotting cascade and toll-like receptor mediated stimulation of the immune system.

Chemotherapeutics are most commonly small molecules, however, their efficacy is hampered by the severe off-target side toxicity, as well as their poor solubility, rapid clearance and limited tumor exposure. Scaffold-small-molecule drug conjugates such as polymer-drug conjugates (PDCs) are macromolecular constructs with pharmacologically activity, which comprises one or more molecules of a small-molecule drug bound to a carrier scaffold (e.g. polyethylene glycol (PEG)).

Such conjugate principle has attracted much attention and has been under investigation for several decades. The majority of conjugates of small-molecule drugs under pre-clinical or clinical development are for oncological indications. However, up-to-date only one drug not related to cancer has been approved (Movantik, a PEG oligomer conjugate of opioid antagonist naloxone, AstraZeneca) for opioid-induced constipation in patients with chronic pain in 2014, which is a non-oncology indication. Translating application of drug-scaffold conjugates into treatment of human subjects provided little clinical success so far. For example, PK1 (N-(2-hydroxypropyl)methacrylamide (HPMA) copolymer doxorubicin; development by Pharmacia, Pfizer) showed great anti-cancer activity in both solid tumors and leukemia in murine models, and was under clinical investigation for oncological indications. Despite that it demonstrated significant reduction of nonspecific toxicity and improved pharmacokinetics in man, improvements in anticancer efficacy turned out to be marginal in patients, and as a consequence further development of PK1 was discontinued.

The failure of scaffold-small-molecule drug conjugates is at least partially attributed to its poor accumulation at the tumor site. For example, while in murine models PK1 showed 45-250 times higher accumulation in the tumor than in healthy tissues (liver, kidney, lung, spleen, and heart), accumulation in tumor was only observed in a small subset of patients in the clinical trial. A potential solution to the aforementioned problems is application of nanoparticle systems for drug delivery such as liposomes. Liposomes are sphere-shaped vesicles consisting of one or more phospholipid bilayers, which are spontaneously formed when phospholipids are dispersed in water. The amphiphilicity characteristics of the phospholipids provide it with the properties of self-assembly, emulsifying and wetting characteristics, and these properties can be employed in the design of new drugs and new drug delivery systems. Drug encapsulated in a liposomal delivery system may convey several advantages over a direct administration of the drug, such as an improvement and control over pharmacokinetics and pharmacodynamics, tissue targeting property, decreased toxicity and enhanced drug activity. An example of such success is liposome-encapsulated form of a small molecule chemotherapy agent doxorubicin (Doxil: a pegylated liposome-encapsulated form of doxorubicin; Myocet: a non-pegylated liposomal doxorubicin), which have been approved for clinical use.

Therefore, a solution still needs to be found that allows for drug therapies such as anti-tumor therapies and anti-auto-immune disease therapies (e.g. rheumatoid arthritis treatment options), applicable for non-systemic use when desired, wherein the drug has for example an acceptable safety profile, little to no off-target activity, sufficient efficacy, sufficiently low clearance rate from the patient's body, a sufficiently wide therapeutic window, etc.

### SUMMARY OF THE INVENTION

The current invention relates to saponin derivatives and conjugates based on such saponin derivatives, wherein the wild-type or natural saponin on which the derivatives and conjugates are based, has at least an aldehyde functional group in the aglycone core structure. According to the invention, typically, the saponins on which the derivatised saponins and saponin-comprising conjugates are based, are penta-cyclic triterpene saponins of the 12,13-dehydrooleanane type, preferably with an aldehyde function in position C-23 of the aglycone core structure of the saponin, and preferably the saponin is SO1861.

Surprisingly, the inventors have found that saponin derivatives according to the invention, wherein the aldehyde functional group is transformed to a hydrazone functional group according to formula (I)

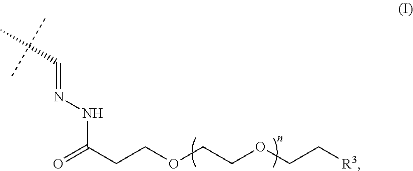

wherein n is an integer selected from 0-15,
R³ is a linker according to formula (III):

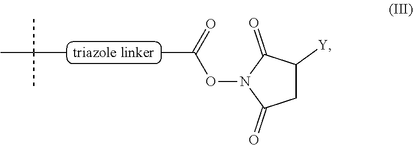

wherein Y is H or SO₂ONa,
at least have one, preferably two, more preferably all three, benefit(s) selected from:
(i) a reduced toxicity when cell viability is considered of cells contacted with the saponin derivatives;
(ii) activity when potentiation of e.g. toxin cytotoxicity or BNA mediated gene silencing is considered (without wishing to be bound by any theory: relating to similar or improved endosomal escape enhancing activity of the modified saponin compared to the activity of the wild-type (natural) saponin); and/or
(iii) reduced hemolytic activity,
when compared with the toxicity, activity and haemolytic activity of unmodified saponin. Therewith, the inventors provide saponin derivatives with an improved therapeutic window, since the ratio between 1050 values for cell toxicity and e.g. 1050 values for toxin potentiation or 1050 values for gene silencing is increased, and/or since the ratio between 1050 values for saponin haemolytic activity and e.g. 1050 values for toxin potentiation or 1050 values for gene silencing is increased.

An aspect of the invention relates to saponin derivatives, wherein the aldehyde functional group is transformed to a hydrazone functional group according to formula (I)

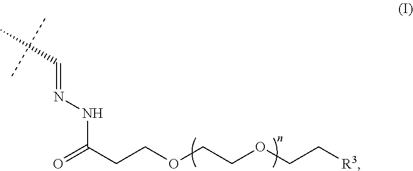

wherein n is an integer selected from 0-15,
R³ is a linker according to formula (III):

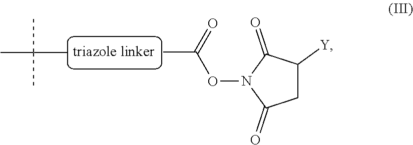

wherein Y is H or SO₂ONa.

An aspect of the invention relates to saponin derivatives, wherein the aldehyde functional group is transformed to a hydrazone functional group according to formula (I)

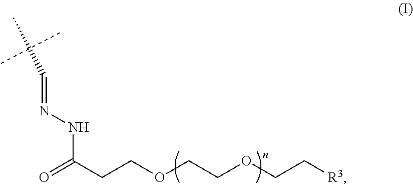

wherein n is an integer preferably selected from 0-15, and R³ is OH. Such saponin derivatives comprise a hydrazone functional group that is covalently linked to a polyethylene glycol (PEG) based on the structure HO—CH₂—CH₂—(O—CH₂—CH₂)ₙ—OH wherein n is an integer preferably selected from 0-15, more preferably 1-13 or 2-12 or 3-11 or 4-10.

Furthermore, the inventors have found that saponin derivatives according to the invention, wherein the aldehyde functional group is transformed to a hydrazone functional group according to formula (I)

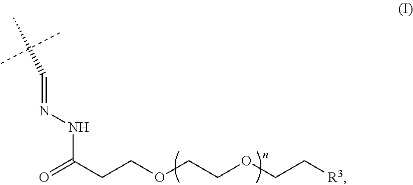

wherein n is an integer selected from 0-15, and
R³ is azide or OH, or R³ is a cyclooctyne moiety, are valuable intermediate saponin derivatives towards the synthesis of saponin derivatives according to the invention, which saponin derivatives have a reduced toxicity when cell viability is considered of cells contacted with the saponin derivatives, have activity when potentiation of e.g. toxin cytotoxicity or BNA mediated gene silencing is considered (without wishing to be bound by any theory: relating to similar or improved endosomal escape enhancing activity of the modified saponin) and/or have reduced hemolytic activity, when compared with the toxicity, activity and haemolytic activity of unmodified saponin.

An embodiment of the invention is a saponin derivative, wherein the aldehyde functional group is transformed to a hydrazone functional group according to formula (I)

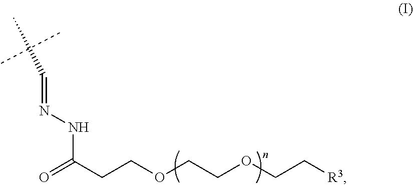

wherein n is an integer selected from 0-15,
R³ is azide or R³ is OH, or R³ is a cyclooctyne moiety. It is part of the invention that when the saponin derivative is a derivative of SO1861, said SO1861 derivative is not a derivative wherein the aldehyde functional group is transformed to a hydrazone functional group according to formula (I)

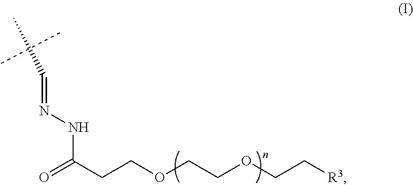

wherein n is 3 and R³ is azide. That is to say, the saponin derivative is not the molecule according to Formula (IV):

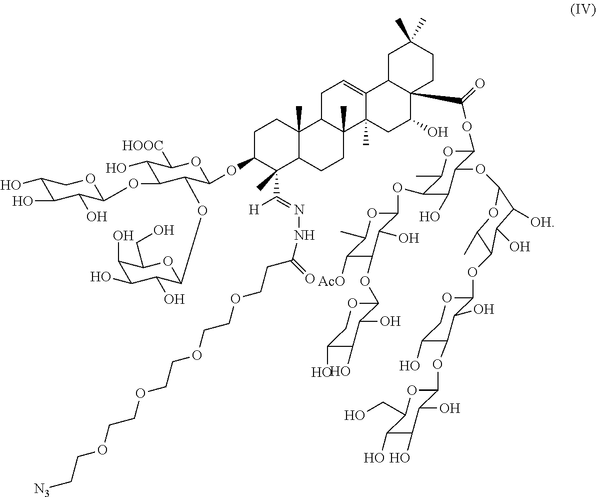

An embodiment of the invention is a saponin derivative, wherein the aldehyde functional group is transformed to a hydrazone functional group according to formula (I)

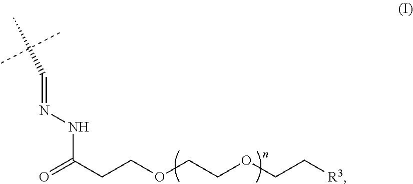

wherein n is an integer selected from 0-15,
R³ is azide or R³ is OH, or R³ is a cyclooctyne moiety, with the proviso that if the saponin derivative is an SO1861 derivative, n is not 3 if R³ is azide, and R³ is not azide if n is 3.

An embodiment of the invention is a saponin derivative, wherein the aldehyde functional group is transformed to a hydrazone functional group according to formula (I)

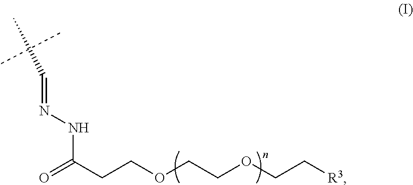

wherein n is an integer selected from 0-15,
R³ is azide or R³ is OH, or R³ is a cyclooctyne moiety, with the proviso that if the saponin derivative is an SO1861 derivative, said SO1861 derivative is not an SO1861 derivative with structure according to Formula (IV):

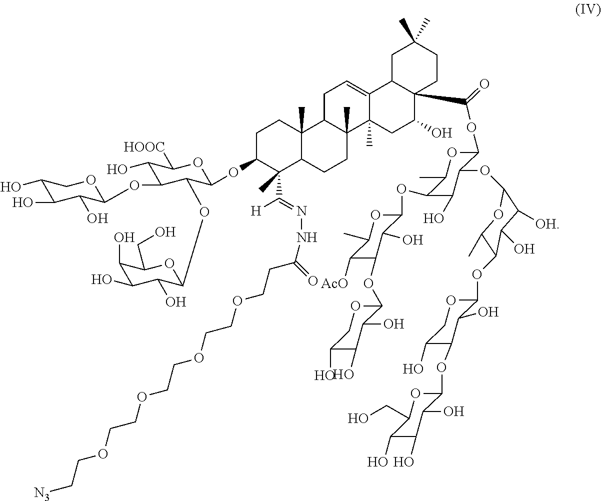

An aspect of the invention relates to a first pharmaceutical composition comprising the saponin derivative according to the invention and optionally a pharmaceutically acceptable excipient and/or diluent.

An aspect of the invention relates to a first pharmaceutical combination comprising:
the first pharmaceutical composition of the invention; and
a second pharmaceutical composition comprising any one or more of: a conjugate of a cell-surface molecule binding-molecule and an effector moiety, such as an antibody-effector moiety conjugate, a receptor-ligand—effector moiety conjugate, an antibody-toxin conjugate, a receptor-ligand—toxin conjugate, an antibody-drug conjugate, a receptor-ligand—drug conjugate, an antibody-oligonucleotide conjugate and a receptor-ligand—oligonucleotide conjugate, and optionally comprising a pharmaceutically acceptable excipient and/or diluent. The effector moiety is not a saponin on which the saponin derivative or the saponin conjugate of the invention are based. The effector moiety is not the saponin derivative or the saponin conjugate of the invention.

An aspect of the invention relates to a third pharmaceutical composition comprising the saponin derivative of the invention and further comprising any one or more of: a conjugate of a cell-surface molecule binding-molecule and an effector moiety, an antibody-effector moiety conjugate, a receptor-ligand—effector moiety conjugate, an antibody-toxin conjugate, a receptor-ligand—toxin conjugate, an antibody-drug conjugate, a receptor-ligand—drug conjugate, an antibody-nucleic acid conjugate or a receptor-ligand—nucleic acid conjugate, and optionally comprising a pharmaceutically acceptable excipient and/or diluent. The effector moiety is not a saponin on which the saponin derivative or the saponin conjugate of the invention are based. The effector moiety is not the saponin derivative or the saponin conjugate of the invention.

An aspect of the invention relates to the first pharmaceutical composition of the invention, the first pharmaceutical combination comprising the first pharmaceutical composition of the invention or the third pharmaceutical composition of the invention, for use as a medicament.

An aspect of the invention relates to the first pharmaceutical composition of the invention, the first pharmaceutical combination comprising the first pharmaceutical composition of the invention or the third pharmaceutical composition of the invention, for use in the treatment or prophylaxis of a cancer or an auto-immune disease.

An aspect of the invention relates to an in vitro or ex vivo method for transferring a molecule from outside a cell to inside said cell, preferably into the cytosol of said cell, comprising the steps of:
a) providing a cell;
b) providing the molecule for transferring from outside the cell into the cell provided in step a);
c) providing a saponin derivative according to the invention;
d) contacting the cell of step a) in vitro or ex vivo with the molecule of step b) and the saponin derivative of step c), therewith establishing the transfer of the molecule from outside the cell into said cell.

An aspect of the invention relates to a saponin conjugate comprising a cell-surface molecule binding-molecule such as a first proteinaceous molecule (‘proteinaceous molecule 1’) or a second proteinaceous molecule (‘proteinaceous molecule 2’) that is covalently bound to the saponin derivative according to the invention.

An aspect of the invention relates to a second pharmaceutical combination comprising:
a) a fourth pharmaceutical composition comprising the saponin conjugate according to the invention and optionally a pharmaceutically acceptable excipient and/or diluent, wherein the proteinaceous molecule 1 and the proteinaceous molecule 2 comprise a same first binding site for binding to a first epitope of a first cell-surface molecule, or wherein the proteinaceous molecule 1 comprises the first binding site for binding to a first epitope of a first cell-surface molecule and the proteinaceous molecule 2 comprises a second binding site for binding to a second epitope of a second cell-surface molecule, wherein the first binding site and the second binding site are different, and optionally wherein the first cell surface molecule and the second cell surface molecule are present on the same cell, preferably the first cell surface molecule and the second cell surface molecule are present on the same cell; and
b) a fifth pharmaceutical composition comprising a conjugate comprising a cell-surface molecule binding-molecule, such as a third proteinaceous molecule (′proteinaceous molecule 3′), and an effector moiety, wherein the proteinaceous molecule 3 is the same or different from the proteinaceous molecule 1 and the proteinaceous molecule 2 present in the saponin conjugate, the proteinaceous molecule 3 comprising a third binding site for binding to a third epitope of a third cell-surface molecule, wherein the third cell-surface molecule, if different from the first cell surface molecule and/or the second cell surface molecule, is present on the same cell as the first cell surface molecule and the second cell surface molecule,
the fifth pharmaceutical composition optionally further comprising a pharmaceutically acceptable excipient and/or diluent. The effector moiety is not a saponin on which the saponin derivative or the saponin conjugate of the invention are based. The effector moiety is not the saponin derivative or the saponin conjugate of the invention.

An aspect of the invention relates to a third pharmaceutical combination comprising:
a) a fourth pharmaceutical composition comprising the saponin conjugate according to the invention and optionally a pharmaceutically acceptable excipient and/or diluent, wherein the proteinaceous molecule 1 and the proteinaceous molecule 2 comprise a same first binding site for binding to a first epitope of a first cell-surface molecule, or wherein the proteinaceous molecule 1 comprises the first binding site for binding to a first epitope of a first cell-surface molecule and the proteinaceous molecule 2 comprises a second binding site for binding to a second epitope of a second cell-surface molecule, wherein the first binding site and the second binding site are different, and optionally wherein the first cell surface molecule and the second cell surface molecule are present on the same cell, preferably the first cell surface molecule and the second cell surface molecule are present on the same cell; and
b) a sixth pharmaceutical composition comprising a conjugate comprising a cell-surface molecule binding-molecule, such as a fourth proteinaceous molecule (‘proteinaceous molecule 4’), and an effector moiety, wherein the proteinaceous molecule 4 comprises the first binding site for binding to the first epitope on the cell-surface molecule of (a), the sixth pharmaceutical composition optionally further comprising a pharmaceutically acceptable excipient and/or diluent,
wherein the first binding site of the proteinaceous molecule 1 or proteinaceous molecule 2 and the first binding site of the proteinaceous molecule 4 are the same, and wherein the first cell-surface molecule and the first epitope on the first cell-surface molecule, to which the proteinaceous molecule 1 or the proteinaceous molecule 2 can bind, and the first cell-surface molecule and the first epitope on the first cell-surface molecule, to which the proteinaceous molecule 4 can bind, are the same. The effector moiety is not a saponin on which the saponin derivative or the saponin conjugate of the invention are based. The effector moiety is not the saponin derivative or the saponin conjugate of the invention.

An aspect of the invention relates to a fourth pharmaceutical combination comprising:
a) the fourth pharmaceutical composition according to the invention; and
b) the sixth pharmaceutical composition according to the invention.

An aspect of the invention relates to a seventh pharmaceutical composition comprising the saponin conjugate according to the invention and the conjugate comprising proteinaceous molecule 3 and an effector moiety, or the conjugate comprising proteinaceous molecule 4 and an effector moiety, and optionally further comprising a pharmaceutically acceptable excipient and/or diluent. The effector moiety is not a saponin on which the saponin derivative or the saponin conjugate of the invention are based. The effector moiety is not the saponin derivative or the saponin conjugate of the invention.

An aspect of the invention relates to the second pharmaceutical combination or third pharmaceutical combination or fourth pharmaceutical combination according to the invention or the seventh pharmaceutical composition according to the invention, for use as a medicament.

An aspect of the invention relates to the second pharmaceutical combination or third pharmaceutical combination or fourth pharmaceutical combination according to the invention or the seventh pharmaceutical composition according to the invention, for use in the treatment or prevention of a cancer or of an autoimmune disease, such as rheumatoid arthritis.

### Definitions

The term “saponin” has its regular scientific meaning and here refers to a group of amphipatic glycosides which comprise one or more hydrophilic glycone moieties combined with a lipophilic aglycone core which is a sapogenin. The saponin may be naturally occurring (‘wild-type’) or synthetic (i.e. non-naturally occurring). The term “saponin” includes naturally-occurring saponins, derivatives of naturally-occurring saponins as well as saponins synthesized de novo through chemical and/or biotechnological synthesis routes. Saponin has a triterpene backbone, which is a pentacyclic C30 terpene skeleton, also referred to as sapogenin or aglycone. Within the context of the invention saponin is not considered an effector molecule nor an effector moiety in the conjugates according to the invention. Thus, in the conjugates comprising a saponin and an effector moiety, the effector moiety is a different molecule than the conjugated saponin.

The term “cell-surface molecule” has its regular scientific meaning and here refers to a molecule that is present and exposed at the outside surface of a cell such as a blood cell or an organ cell, such as a mammalian cell, such as a human cell.

The term “saponin derivative” has its regular scientific meaning and here refers to a saponin, i.e. a modified saponin, which has a chemical modification at a position where previously an aldehyde group was present in the non-derivatised saponin before being subjected to chemical modification for provision of the saponin derivative. For example, the saponin derivative is provided by chemical modification of an aldehyde group, in a saponin upon which the saponin derivative is based, i.e. the saponin is provided and an aldehyde group is chemically modified therewith providing the saponin derivative. For example, the saponin that is derivatised for provision of the saponin derivative is a naturally occurring saponin. Typically, the saponin derivative is a synthetic saponin, typically the saponin derivative is a derivatisation of a natural saponin, and is thus derived from a natural saponin, although a saponin derivative can also be derived from a synthetic saponin which may or may not have a natural counterpart. Typically, the saponin derivative has not a natural counterpart, i.e. the saponin derivative is not produced naturally by e.g. plants or trees. Optionally, the saponin derivative further has one or more chemical modifications at positions where previously any of a carboxyl group, an acetate group and/or an acetyl group was present in the non-derivatised or derivatised saponin before being subjected to chemical modification for provision of the saponin derivative. For example, the saponin derivative is provided by chemical modification of any one or more of an carboxyl group, an acetate group and/or an acetyl group in a saponin upon which the saponin derivative is based, i.e. the saponin is provided and an aldehyde group, a carboxyl group, an acetate group and/or an acetyl group is chemically modified therewith providing the saponin derivative.

The term “mono-desmosidic saponin” has its regular scientific meaning and here refers to a triterpenoid saponin containing a single saccharide chain bound to the aglycone core, wherein the saccharide chain consists of one or more saccharide moieties.

The term “bi-desmosidic saponin” has its regular scientific meaning and here refers to a triterpenoid saponin containing two saccharide chains bound to the aglycone core, wherein each of the two saccharide chains consists of one or more saccharide moieties.

The term “triterpenoid saponin” has its regular scientific meaning and here refers to a saponin having a triterpenoid-type of aglycone core structure. The triterpenoid saponin differs from a saponin based on a steroid glycoside such as sapogenol in that such saponin comprising steroid glycoside has a steroid core structure, and the triterpenoid saponin differs from a saponin based on an alkaloid glycoside such as tomatidine in that such saponin comprising alkaloid glycoside has a alkaloid core structure.

The term “conjugate” has its regular scientific meaning and here refers to at least a first molecule that is covalently bound through chemical bonds to at least a second molecule, therewith forming a covalently coupled assembly comprising or consisting of the first molecule and the second molecule. Typical conjugates are an ADC, an AOC, and SO1861-EMCH (EMCH linked to the aldehyde group of the aglycone core structure of the saponin SO1861).

The term “linker” has its regular scientific meaning, and linkers are commonly known in the art of bioconjugation. Common linkers are for example described in G. T. Hermanson (Bioconjugation Techniques, Third edition, Elsevier, 2013, ISBN: 978-0-12-382239-0). Here, the term linker refers to a chemical moiety, which is suitable for covalently attaching (binding) a first molecule, such as a saponin, to another molecule, e.g. to a (proteinaceous) ligand or to an effector molecule or to a scaffold, for example composed of or comprising amino-acid residues, nucleic acids, etc. Typically, the linker comprises a chain of atoms linked by chemical bonds. Any linker molecule or linker technology known in the art can be used in the present disclosure. Where indicated, the linker is a linker for covalently binding of molecules through a chemical group on such a molecule suitable for forming a covalent linkage or bond with the linker. The linker may be a non-cleavable linker, e.g., the linker is stable in physiological conditions. The linker may be a cleavable linker, e.g. a linker that is cleavable, in the presence of an enzyme or at a particular pH range or value, or under physiological conditions such as intracellular conditions in the endosomes such as the late endosomes and the lysosomes of mammalian cells such as human cells. Exemplary linkers that can be used in the context of the present disclosure include, but are not limited to, N-ε-maleimidocaproic acid hydrazide (EMCH), succinimidyl 3-(2-pyridyldithio)propionate or 3-(2-Pyridyldithio)propionic acid N-hydroxysuccinimide ester (SPDP), PEG-azide, and 1-[Bis(dimethylamino)methylene]-1H-1,2,3-triazolo[4,5-b]pyridinium 3-oxid hexafluorophosphate (HATU), and a hydrazone functional group according to formula (I)

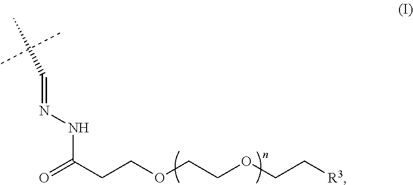

wherein n is an integer selected from 0-15,
R³ is azide or R³ is OH, or
R³ is a cyclooctyne moiety selected from the group consisting of cyclooctyne moieties (II)a-d

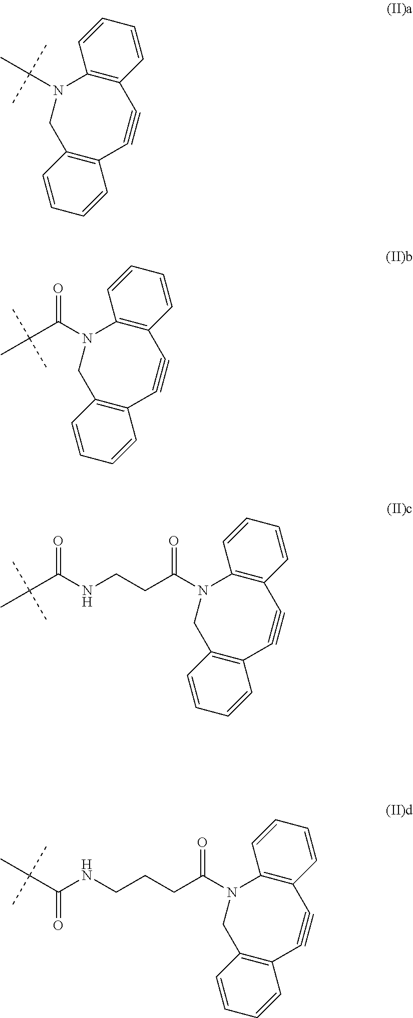

or,
R³ is a linker according to formula (III):

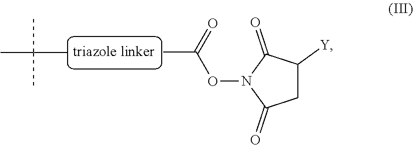

wherein Y is H or SO₂ONa,
and the linker according to formula (III) is selected from the group consisting of linkers (IV)a-j:

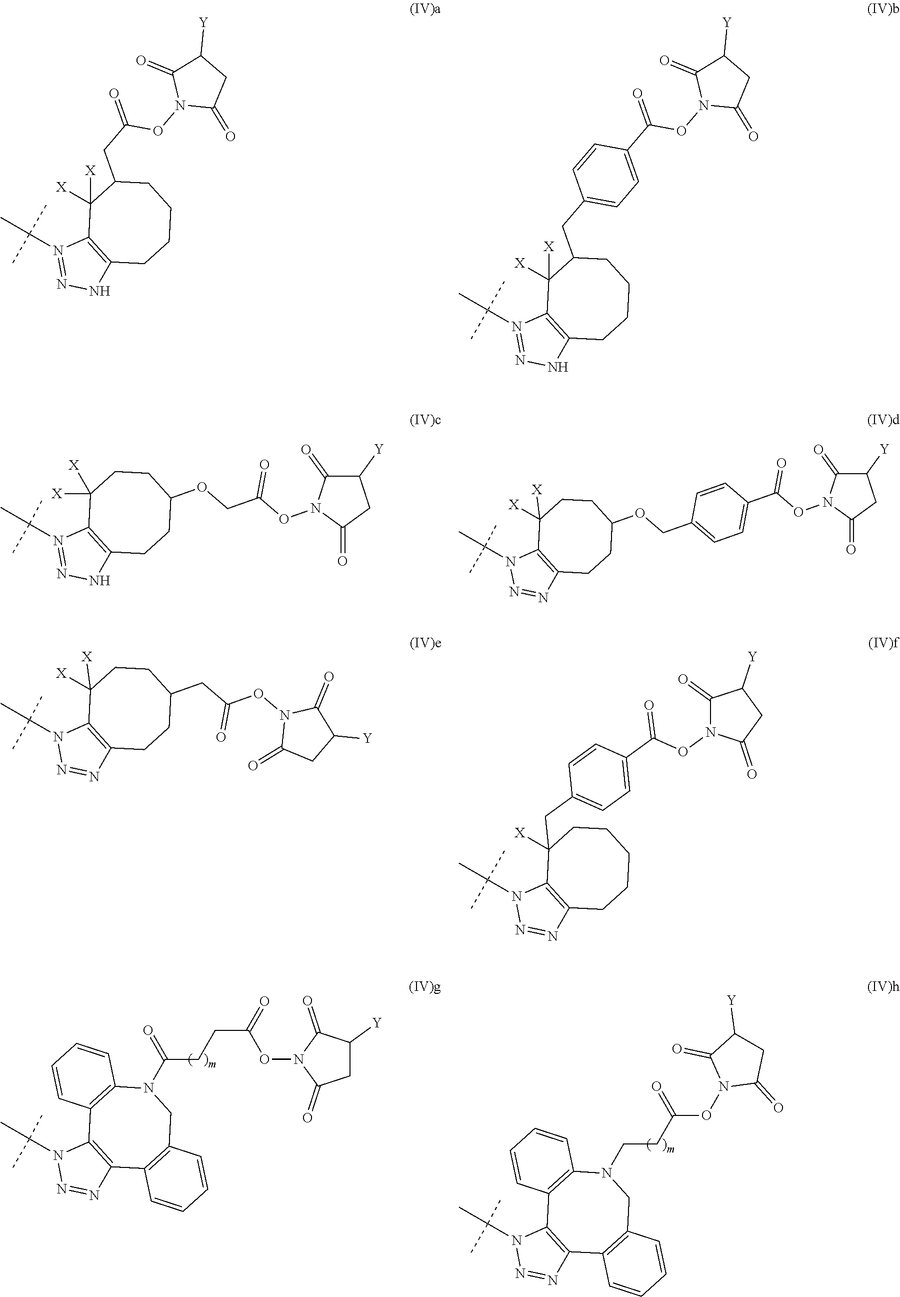

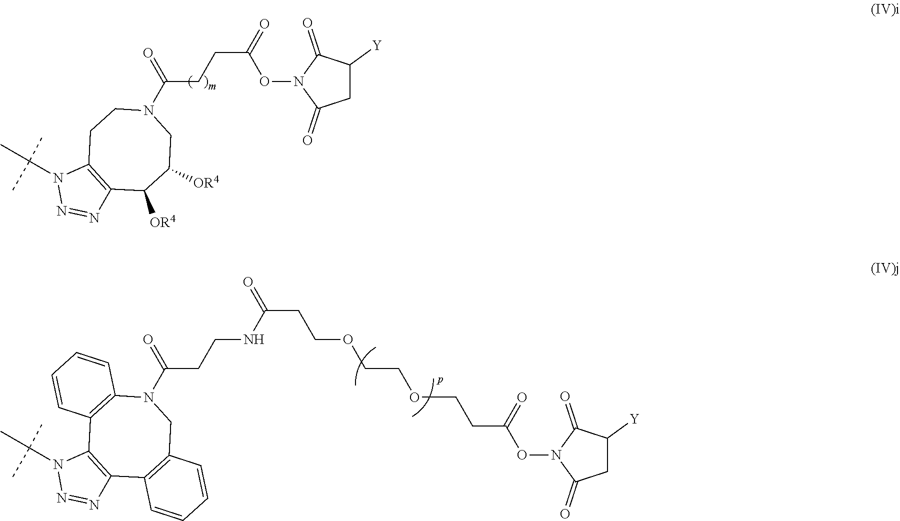

wherein X is H or F, preferably F;
m is 1 or 2;
p is an integer selected from 0-5, preferably 1→4, more preferably 2-3; and
R⁴ is a C1-C3 alkyl chain, preferably a C1 alkyl chain.

The term “tumor cell-specific surface molecule” and the term “tumor cell-specific receptor” have their regular scientific meaning and here refer to a molecule or a receptor that is expressed and exposed at the surface of a tumor cell and not at the surface of a healthy, non-cancerous cell, or is expressed at the surface of a healthy, non-cancerous cell to a lower extent than the level of expression (number of molecules or receptors) at the surface of the tumor cell.

The term “oligonucleotide” has its regular scientific meaning and here refers to a string of two or more nucleotides, i.e. an oligonucleotide is a short oligomer composed of ribonucleotides or deoxyribonucleotides. Examples are RNA and DNA, and any modified RNA or DNA, such as a string of nucleic acids comprising a nucleotide analogue such as a bridged nucleic acid (BNA), also known as locked nucleic acid (LNA), wherein the nucleotide is a ribonucleotide or a deoxyribonucleotide.

The term “payload” has its regular scientific meaning and here refers to a biologically active molecule such as for example a cytotoxic (anti-cancer) drug molecule.

The term “proteinaceous” has its regular scientific meaning and here refers to a molecule comprising at least two amino acid residues linked via a peptide bond with each other so that the molecule is of, relates to, resembles, or is a polypeptide or a protein, meaning that the molecule possesses, to some degree, the physicochemical properties characteristic of a protein, is of protein, relating to protein, containing protein, pertaining to protein, consisting of protein, resembling protein, or being a protein. The term “proteinaceous” as used in for example ‘proteinaceous molecule’ refers to the presence of at least two amino acid residues linked via a peptide bond with each other so that at least a part of the molecule that resembles or is a protein, wherein ‘protein’ is to be understood to include a chain of amino-acid residues at least two residues long, thus including a peptide, a polypeptide and a protein and an assembly of proteins or protein domains. In the proteinaceous molecule, the at least two amino-acid residues are for example linked via (an) amide bond(s), such as (a) peptide bond(s). In the proteinaceous molecule, the amino-acid residues are natural amino-acid residues and/or artificial amino-acid residues such as modified natural amino-acid residues. It is preferred that a proteinaceous molecule is a molecule comprising at least two amino-acid residues, preferably between 2 and about 2.000 amino-acid residues. Also preferred is a proteinaceous molecule that is a molecule comprising from 2 to 20 (typical for a peptide) amino acids. Also preferred is a proteinaceous molecule that is a molecule comprising from 21 to 1.000 amino acid residues (typical for a polypeptide, a protein, a protein domain, such as an antibody, a Fab, an scFv, a ligand for a receptor such as EGF). Preferably, the amino-acid residues are (typically) linked via (a) peptide bond(s). According to the invention, said amino-acid residues are or comprise (modified) (non-)natural amino acid residues.

The term “binding molecule” has its regular scientific meaning and here refers to a molecule capable of specifically binding to another molecule such as a cell-surface molecule, e.g. a cell-surface receptor. Typical binding molecules are peptides, proteins, non-protein molecules, cell-surface receptor ligands, protein ligands, that can bind to e.g. a protein, a lipid, a (poly)saccharide, such as a cell-surface receptor or a cell-surface molecule. “Specifically binding” here refers to specific and selective binding with higher affinity than non-specific background binding.

The term “moiety” has its regular scientific meaning and here refers to a molecule that is bound, linked, conjugated to a further molecule, linker, assembly of molecules, etc., and therewith forming part of a larger molecule, conjugate, assembly of molecules. Typically, a moiety is an molecule that is covalently bound to another molecule, involving one or more chemical groups initially present on the effector molecule. For example, saporin is a typical effector molecule. As part of an antibody-drug conjugate, the saporin is a typical effector moiety in the ADC. As part of an antibody-oligonucleotide conjugate, a BNA or an siRNA is a typical effector moiety in the AOC.

The term “aglycone core structure” has its regular scientific meaning and here refers to the aglycone core of a saponin without the one or two carbohydrate antenna or saccharide chains (glycans) bound thereto. For example, quillaic acid is the aglycone core structure for SO1861, QS-7 and QS21. Typically, the glycans of a saponin are mono-saccharides or oligo-saccharides, such as linear or branched glycans.

The term “QS21”, unless further specified, refers to any one of the isomers of QS21. As will be understood by the skilled person, a typical natural extract comprising QS21 will comprise a mixture of the different isomers of QS21. However, through purification or (semi-)synthetic routes, a single isomer can be isolated.

The term “Saponinum album” has its normal meaning and here refers to a mixture of saponins produced by Merck KGaA (Darmstadt, Germany) containing saponins from Gypsophila paniculata and Gypsophila arostii, containing SA1657 and mainly SA1641.

The term “Quillaja saponin” has its normal meaning and here refers to the saponin fraction of Quillaja saponaria and thus the source for all other QS saponins, mainly containing QS-18 and QS-21.

“QS-21” or “QS21” has its regular scientific meaning and here refers to a mixture of QS-21 A-apio (˜63%), QS-21 A-xylo (˜32%), QS-21 B-apio (˜3.3%), and QS-21 B-xylo (˜1.7%).

Similarly, “QS-21A” has its regular scientific meaning and here refers to a mixture of QS-21 A-apio (˜65%) and QS-21 A-xylo (˜35%).

Similarly, “QS-21 B” has its regular scientific meaning and here refers to a mixture of QS-21 B-apio (˜65%) and QS-21 B-xylo (˜35%).

The term “Quit-A” refers to a commercially available semi-purified extract from Quillaja saponaria and contains variable quantities of more than 50 distinct saponins, many of which incorporate the triterpene-trisaccharide substructure Gal-(1→2)-[Xyl-(1→3)]-GlcA- at the C-3beta-OH group found in QS-7, QS-17, QS18, and QS-21. The saponins found in Quil-A are listed in van Setten (1995), Table 2 [Dirk C. van Setten, Gerrit van de Werken, Gijsbert Zomer and Gideon F. A. Kersten, Glycosyl Compositions and Structural Characteristics of the Potential Immuno-adjuvant Active Saponins in the Quillaja saponaria Molina Extract Quil A, RAPID COMMUNICATIONS IN MASS SPECTROMETRY, VOL. 9,660-666 (1995)]. Quil-A and also Quillaja saponin are fractions of saponins from Quillaja saponaria and both contain a large variety of different saponins with largely overlapping content. The two fractions differ in their specific composition as the two fractions are gained by different purification procedures.

The term “QS1861” and the term “QS1862” refer to QS-7 and QS-7 api. QS1861 has a molecular mass of 1861 Dalton, QS1862 has a molecular mass of 1862 Dalton. QS1862 is described in Fleck et al. (2019) in Table 1, row no. 28 [Juliana Deise Fleck, Andresa Heemann Betti, Francini Pereira da Silva, Eduardo Artur Troian, Cristina Olivaro, Fernando Ferreira and Simone Gasparin Verza, Saponins from Quillaja saponaria and Quillaja brasiliensis: Particular Chemical Characteristics and Biological Activities, Molecules 2019, 24, 171; doi:10.3390/molecules24010171]. The described structure is the api-variant QS1862 of QS-7. The molecular mass is 1862 Dalton as this mass is the formal mass including proton at the glucuronic acid. At neutral pH, the molecule is deprotonated. When measuring in mass spectrometry in negative ion mode, the measured mass is 1861 Dalton.

The term “saccharide chain” has its regular scientific meaning and here refers to any of a glycan, a carbohydrate antenna, a single saccharide moiety (mono-saccharide) or a chain comprising multiple saccharide moieties (oligosaccharide, polysaccharide). The saccharide chain can consist of only saccharide moieties or may also comprise further moieties such as any one of 4E-Methoxycinnamic acid, 4Z-Methoxycinnamic acid, and 5-O-[5-O-Ara/Api-3,5-dihydroxy-6-methyl-octanoyl]-3,5-dihydroxy-6-methyl-octanoic acid), such as for example present in QS-21.

The term “transformation” has its regular scientific meaning and here refers to the chemical transformation or modification of a first functional group or first chemical group or first chemical moiety such that a second functional group or second chemical group or second chemical moiety is provided. An example is the transformation of an aldehyde group carbonyl group into a hydrazone functional group through reaction with an hydrazine.

The term “Api/Xyl-” or “Api- or Xyl-” in the context of the name of a saccharide chain has its regular scientific meaning and here refers to the saccharide chain either comprising an apiose (Api) moiety, or comprising a xylose (Xyl) moiety.

The term “oligonucleotide” has its regular scientific meaning and here refers to amongst others any natural or synthetic string of nucleic acids encompassing DNA, modified DNA, RNA, mRNA, modified RNA, synthetic nucleic acids, presented as a single-stranded molecule or a double-stranded molecule, such as a BNA, an antisense oligonucleotide (ASO, AON), a short or small interfering RNA (siRNA; silencing RNA), an anti-sense DNA, anti-sense RNA, etc. The term “oligonucleotide” here also refers to a string of two or more nucleotides, i.e., an oligonucleotide is a short oligomer composed of ribonucleotides or deoxyribonucleotides. Examples are RNA and DNA, and any modified RNA or DNA, such as a string of nucleic acids comprising a nucleotide analogue such as a bridged nucleic acid (BNA), also known as locked nucleic acid (LNA) or a 2′-0,4′-C-aminoethylene or a 2′-0,4′-C-aminomethylene bridged nucleic acid (BNANc), wherein the nucleotide is a ribonucleotide or a deoxyribonucleotide.

The term “antibody” as used herein is used in the broadest sense, which may refer to an immunoglobulin (Ig) defined as a protein belonging to the class IgG, IgM, IgE, IgA, or IgD (or any subclass thereof), or a functional binding fragment or binding domain of an immunoglobulin. In the context of the present invention, a “binding fragment” or a “binding domain” of an immunoglobulin or of an antibody is defined as antigen-binding fragment or -domain or other derivative of a parental immunoglobulin that essentially maintains the antigen binding activity of such parental immunoglobulin. Functional fragments and functional domains are antibodies in the sense of the present invention even if their affinity to the antigen is lower than that of the parental immunoglobulin. “Functional fragments and -domains” in accordance with the invention include, but are not limited to, F(ab′)2 fragments, Fab′ fragments, Fab fragments, scFv, dsFv, single-domain antibody (sdAb), monovalent IgG, scFv-Fc, reduced IgG (rIgG), minibody, diabodies, triabodies, tetrabodies, Fc fusion proteins, nanobodies, variable V domains such as V_{HH}, Vh, and other types of antigen recognizing immunoglobulin fragments and domains. The fragments and domains may be engineered to minimize or completely remove the intermolecular disulphide interactions that occur between the CH1 and CL domains. Functional fragment and—domains offer the advantage of greater tumor penetration because of their smaller size. In addition, the functional fragment or—domain can be more evenly distributed throughout the tumor mass as compared to whole immunoglobulin.

The term “single domain antibody”, or “sdAb”, in short, has its regular scientific meaning and here refers to an antibody fragment consisting of a single monomeric variable antibody domain. In the conjugates of the invention, more than one sdAb can be present, which sdAbs can be the same (multivalent and mono-specific) or can be different (multivalent and/or for example multi-paratope, bi-paratope, multi-specific, bi-specifc). In addition, the more than two sdAbs can for example be a combination of mono-specific and multivalent sdAbs and at least one further sdAb that binds to a different epitope (e.g. multi-specific or bi-paratope).

The antibodies (immunoglobulins) of the present invention may be bi- or multifunctional. For example, a bifunctional antibody has one arm having a specificity for one receptor or antigen, while the other arm recognizes a different receptor or antigen. Alternatively, each arm of the bifunctional antibody may have specificity for a different epitope of the same receptor or antigen of the target cell.

The antibodies (immunoglobulins) of the present invention may be, but are not limited to, polyclonal antibodies, monoclonal antibodies, human antibodies, humanized antibodies, chimeric antibodies, resurfaced antibodies, anti-idiotypic antibodies, mouse antibodies, rat antibodies, rat/mouse hybrid antibodies, llama antibodies, llama heavy-chain only antibodies, heavy-chain only antibodies, and veterinary antibodies. Preferably, the antibody (immunoglobulin) of the present invention is a monoclonal antibody. The resurfaced, chimeric, humanized and fully human antibodies are also more preferred because they are less likely to cause immunogenicity in humans. The antibodies of the ADC of the present invention preferably specifically binds to an antigen expressed on the surface of a cancer cell, an autoimmune cell, a diseased cell, an aberrant cell, while leaving any healthy cell essentially unaltered (e.g. by not binding to such normal cell, or by binding to a lesser extent in number and/or affinity to such healthy cell).

The term “antibody-drug conjugate” or “ADC” has its regular scientific meaning and here refers to any conjugate of an antibody such as an IgG, an immunoglobulin, an immunoglobulin binding fragment, a binding derivative or binding fragment or binding domain of an antibody such as a F(ab′)2 fragment, Fab′ fragment, Fab fragment, scFv, dsFv, single-domain antibody (sdAb), scFv-Fc, reduced IgG (rIgG), minibody, diabody, triabody, tetrabody, Fc fusion protein, nanobody, variable V domain, a single-domain antibody (sdAb), preferably a V_{HH}, multiple V_{H} domains, single-domain antibodies, V_{HH}, or camelid V_{H}, etc., and any molecule that can exert a therapeutic effect when contacted with cells of a subject such as a human patient, such as an active pharmaceutical ingredient, a toxin, an oligonucleotide, an enzyme, a small molecule drug compound, etc.

The term “antibody-oligonucleotide conjugate” or “AOC” has its regular scientific meaning and here refers to any conjugate of an antibody such as an IgG, an immunoglobulin, an immunoglobulin binding fragment, a binding derivative or binding fragment or binding domain of an antibody such as a F(ab′)2 fragment, Fab′ fragment, Fab fragment, scFv, dsFv, single-domain antibody (sdAb), scFv-Fc, reduced IgG (rIgG), minibody, diabody, triabody, tetrabody, Fc fusion protein, nanobody, variable V domain, a single-domain antibody (sdAb), preferably a V_{HH}, multiple V_{H} domains, single-domain antibodies, V_{HH}, or camelid V_{H}, etc., and any oligonucleotide molecule that can exert a therapeutic effect when contacted with cells of a subject such as a human patient, such as an oligonucleotide selected from a natural or synthetic string of nucleic acids encompassing DNA, modified DNA, RNA, mRNA, modified RNA, synthetic nucleic acids, presented as a single-stranded molecule or a double-stranded molecule, such as a BNA, an antisense oligonucleotide (ASO), a short or small interfering RNA (siRNA; silencing RNA), an anti-sense DNA, anti-sense RNA, etc.

The term “effector molecule”, or “effector moiety” when referring to the effector molecule as part of e.g. a covalent conjugate, has its regular scientific meaning and here refers to a molecule or moiety that has an effect on any one or more of a target molecule and/or proximally to any one or more of a target molecule and/or that can selectively bind to any one or more of a target molecule, wherein the target molecules are for example: a protein, a peptide, a carbohydrate, a saccharide such as a glycan, a (phospho)lipid, a nucleic acid such as DNA, RNA, an enzyme, and regulates the biological activity of such one or more target molecule(s). In the conjugate of the invention the effector moiety for example exerts its effect in the cytosol, in the cell nucleus, is delivered intracellularly in the endosome and/or lysosome, and/or is active after exiting or escaping the endosomal-lysosomal pathway. The effector molecule is for example a molecule selected from any one or more of a small molecule such as a drug molecule, a toxin such as a protein toxin, an oligonucleotide such as a BNA, a xeno nucleic acid or an siRNA, an enzyme, a peptide, a protein, or an active fragment or an active domain thereof, or any combination thereof. Thus, for example, an effector molecule or an effector moiety is a molecule or moiety selected from any one or more of a small molecule such as a drug molecule, a toxin such as a protein toxin, an oligonucleotide such as a BNA, a xeno nucleic acid or an siRNA, an enzyme, a peptide, a protein, or any combination thereof, that can selectively bind to any one or more of the target molecules: a protein, a peptide, a carbohydrate, a saccharide such as a glycan, a (phospho)lipid, a nucleic acid such as DNA, RNA, an enzyme, and that upon binding to the target molecule regulates the biological activity of such one or more target molecule(s). For example, an effector moiety is a toxin or an active toxic fragment thereof or an active toxic derivative or an active toxic domain thereof. An effect can include, but is not limited to, biological effect, a therapeutic effect, an imaging effect, and/or a cytotoxic effect. Typically, an effector molecule or moiety can exert a biological effect inside a cell such as a mammalian cell such as a human cell, such as in the cytosol of said cell. At a molecular or cellular level, an effect can include, but is not limited to, promotion or inhibition of the target's activity, labelling of the target, and/or cell death. Typical effector molecules and effector moieties are thus protein toxins, drug molecules, plasmid DNA, toxins such as toxins comprised by antibody-drug conjugates (ADCs), oligonucleotides such as siRNA, BNA, nucleic acids comprised by an antibody-oligonucleotide conjugate (AOC), enzymes. For example, an effector molecule or moiety is a molecule which can act as a ligand that can increase or decrease (intracellular) enzyme activity, gene expression, or cell signalling. The effector moiety is not a saponin on which the saponin derivative or the saponin conjugate of the invention are based. The effector moiety is not the saponin derivative or the saponin conjugate of the invention. Typically, an effector moiety comprised by the conjugate exerts its therapeutic (for example toxic, enzymatic, inhibitory, gene silencing, etc.) effect in the cytosol and/or in the cell nucleus. Typically, the effector moiety is delivered intracellularly in the endosome and/or in the lysosome, and typically the effector moiety is active after exiting or escaping the endosomal-lysosomal pathway.

The term “HSP27” relates to a BNA molecule which silences the expression of HSP27 in the cells.

The term “bridged nucleic acid”, or “BNA” in short, or “locked nucleic acid” or “LNA” in short, or 2′-O,4′-C-aminoethylene or 2′-O,4′-C-aminomethylene bridged nucleic acid (BNANc), has its regular scientific meaning and here refers to a modified RNA nucleotide. A BNA is also referred to as ‘constrained RNA molecule’ or ‘inaccessible RNA molecule’. A BNA monomer can contain a five-membered, six-membered or even a seven-membered bridged structure with a “fixed” C3-endo sugar puckering. The bridge is synthetically incorporated at the 2′, 4′-position of the ribose to afford a 2′, 4′-BNA monomer. A BNA monomer can be incorporated into an oligonucleotide polymeric structure using standard phosphoramidite chemistry known in the art. A BNA is a structurally rigid oligonucleotide with increased binding affinity and stability.

The term antisense oligonucleotide has its regular scientific meaning and may be indicated in short in the description as “AON” or “ASO”.

The terms first, second, third and the like in the description and in the claims, are used for distinguishing between for example similar elements, compositions, constituents in a composition, or separate method steps, and not necessarily for describing a sequential or chronological order. The terms are interchangeable under appropriate circumstances and the embodiments of the invention can operate in other sequences than described or illustrated herein, unless specified otherwise.

The term as used such as in an saponin derivative or an antibody-saponin conjugate or construct comprising a linker, represents ‘labile linker’ which is cleaved under slightly acid conditions (pH<6.6, such as pH 4.0-5.5) in the endosome, endolysosome and in the lysosome of mammalian cells, such as human cells, such as a human tumor cell.

The embodiments of the invention described herein can operate in combination and cooperation, unless specified otherwise.

Furthermore, the various embodiments, although referred to as “preferred” or “e.g.” or “for example” or “in particular” and the like are to be construed as exemplary manners in which the invention may be implemented rather than as limiting the scope of the invention.

The term “comprising”, used in the claims, should not be interpreted as being restricted to for example the elements or the method steps or the constituents of a compositions listed thereafter; it does not exclude other elements or method steps or constituents in a certain composition. It needs to be interpreted as specifying the presence of the stated features, integers, (method) steps or components as referred to, but does not preclude the presence or addition of one or more other features, integers, steps or components, or groups thereof. Thus, the scope of the expression “a method comprising steps A and B” should not be limited to a method consisting only of steps A and B, rather with respect to the present invention, the only enumerated steps of the method are A and B, and further the claim should be interpreted as including equivalents of those method steps. Thus, the scope of the expression “a composition comprising components A and B” should not be limited to a composition consisting only of components A and B, rather with respect to the present invention, the only enumerated components of the composition are A and B, and further the claim should be interpreted as including equivalents of those components.

In addition, reference to an element or a component by the indefinite article “a” or “an” does not exclude the possibility that more than one of the element or component are present, unless the context clearly requires that there is one and only one of the elements or components. The indefinite article “a” or “an” thus usually means “at least one”.

### BRIEF DESCRIPTION OF THE DRAWINGS

The present invention will be discussed in more detail below, with reference to the attached drawings, in which

FIG. 1: Cell killing assay (MTS) of SO1861-L-NHS+5 pM EGF-dianthin on HeLa (A) and A431 (B) cell lines

FIG. 2: Cell killing assay (MTS) of SO1861-L-NHS+10 pM Cetuximab-saporin on HeLa (A) and A431 (B) cell lines

FIG. 3: Cell killing assay (MTS) of SO1861-L-NHS+50 pM Trastuzumab-saporin on HeLa (A) and A431 (B) cell lines. The figure legend for FIG. 3B is the same as the Figure legend for FIG. 3A.

FIG. 4: Cell killing assay (MTS) of SO1861-L-NHS on HeLa (A) and A431 (B) cell lines. The figure legend for FIG. 4B is the same as the Figure legend for FIG. 4A.

FIG. 5: Hemolytic activity of SO1861-L-NHS on human red blood cell. Note: SPT001 is SO1861. A.D.: aqua dest.

FIG. 6: graphical representation of mAb-L-NHS-SO1861 (DAR4) (note: SPT001 is SO1861)

FIG. 7: EGFR/CD71 targeted cell killing. A-D) Cetuximab-(L-NHS-SO1861)^{4eq, 6eq, 8eq, 9.4 eq, 18.5 eq, 33.4eq} titration or Cetuximab-(EMCH-SO1861)⁴ titration+fixed concentration 10 pM CD71mab-saporin and controls on A431 (A, C) cells (EGFR⁺⁺/CD71⁺) and A2058 (B, D) cells (EGFRICD71⁺). The figure legend for FIG. 7A is the same as the Figure legend for FIG. 7B. The figure legend for FIG. 7D is the same as the Figure legend for FIG. 7C.

FIG. 8: HER2/CD71 targeted cell killing. A-E) Trastuzumab-(L-NHS-SO1861)^{4 eq, 6 eq, 8 eq, 8.6 eq, 16.8 eq, 33.3 eq} titration or Trastuzumab-(EMCH-SO1861) 4 titration+fixed concentration 10 pM CD71mab-saporin and controls on SK-BR3 cells (HER2⁺⁺/CD71⁺) (A, B), JIMT-1 cells (HER2⁻/CD71⁺) (C, E) and MDA-MB-468 (HER2⁻/CD71⁺) (D, F). The figure legend for FIG. 8D is the same as the FIG. legend for FIG. 8C. The figure legend for FIG. 8E is the same as the Figure legend for FIG. 8F.

FIG. 9: Synthesis of molecule 23 (saponin according to formula (IV))

FIG. 10: Synthesis of molecule 25 (saponin according to formula (VIII))

FIG. 11: Cell killing assay (MTS) of various cell lines treated with 5 pM EGFdianthin, 10 pM Cetuximab-saporin, 50 pM Trastuzumab-saporin or 10 pM CD71-saporin

### DETAILED DESCRIPTION

The present invention will be described with respect to particular embodiments but the invention is not limited thereto but only by the claims.

Saponin Derivatives

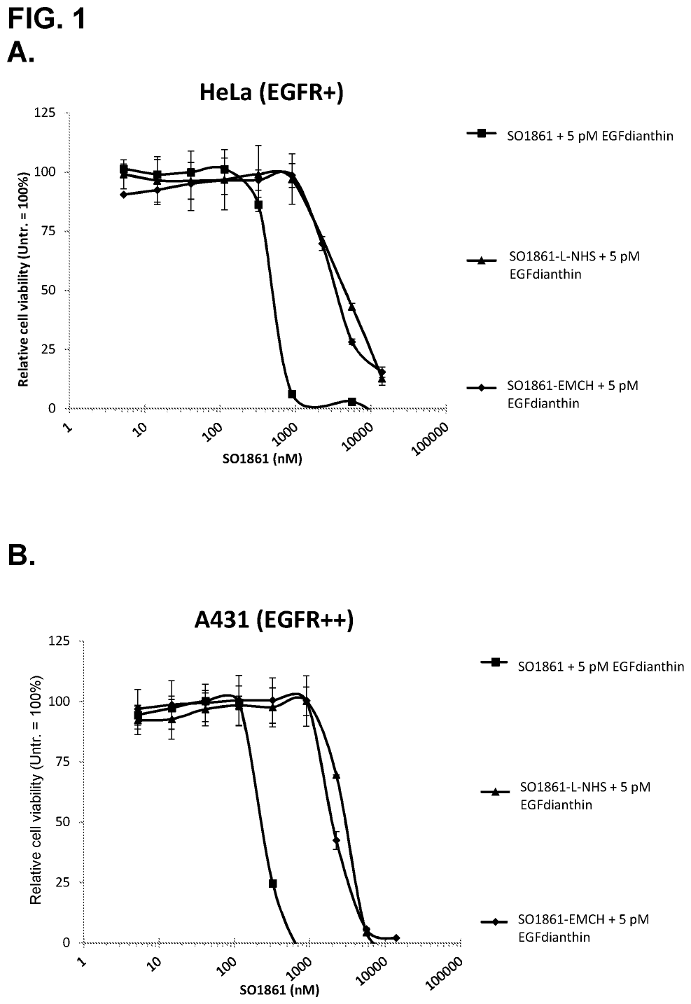

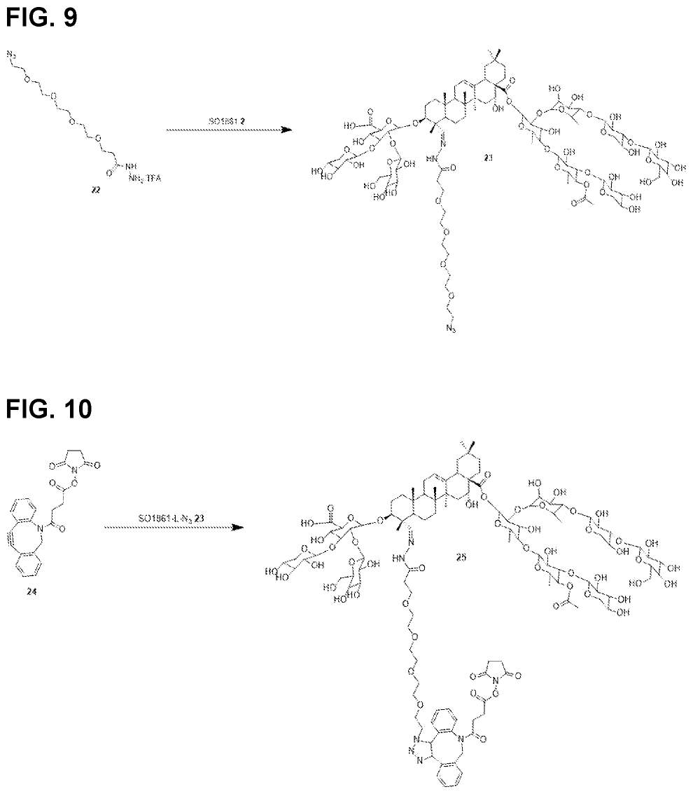

Surprisingly, the inventors have found that saponin derivatives based on a saponin comprising a triterpene aglycone core structure and at least one of a first saccharide chain ‘R¹’ and a second saccharide chain ‘R²’ linked to the aglycone core structure, wherein the saponin derivative comprises an aglycone core structure comprising an aldehyde functional group which has been derivatised, wherein the aldehyde functional group is transformed to a hydrazone functional group according to formula (I)

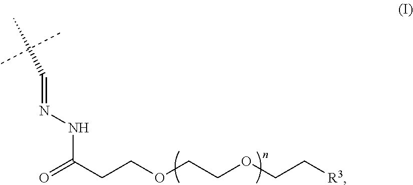

wherein n is an integer selected from 0-15,
R³ is a linker according to formula (III):

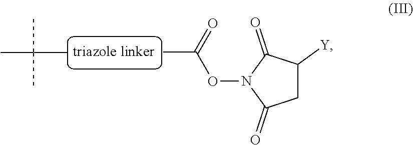

wherein Y is H or SO₂ONa,
and the linker according to formula (III) is selected from the group consisting of linkers (IV)a-j:

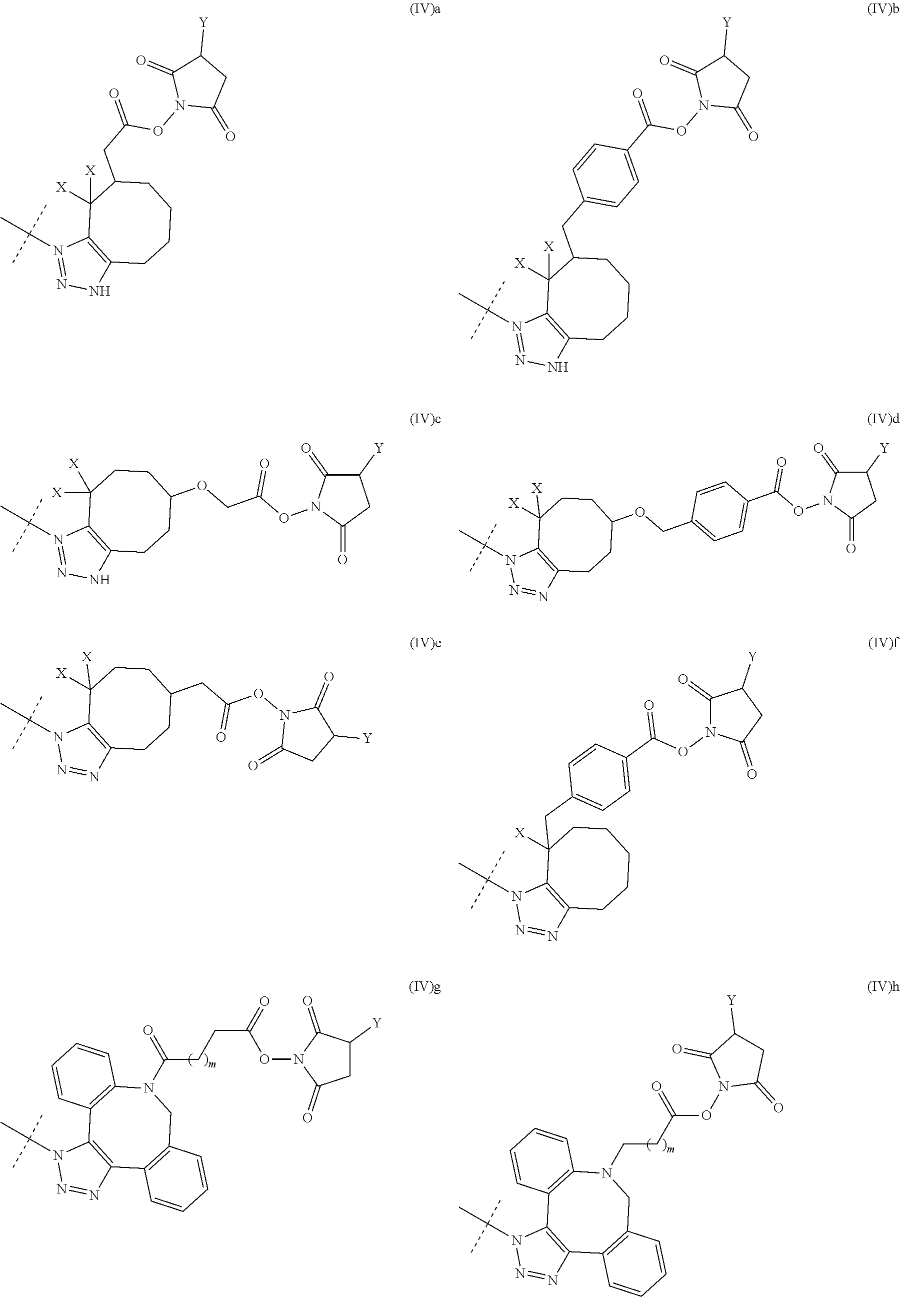

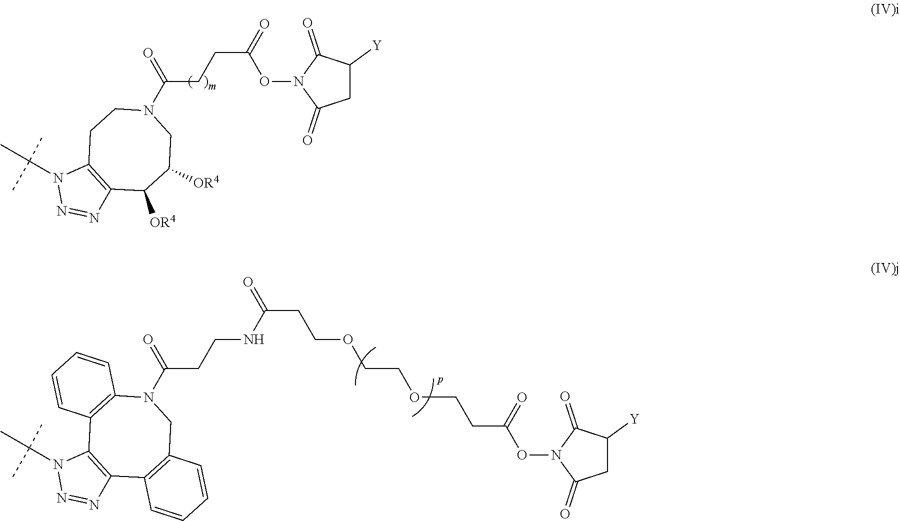

wherein X is H or F, preferably F;
m is 1 or 2;
p is an integer selected from 0-5, preferably 1→4, more preferably 2-3; and
R⁴ is a C1-C3 alkyl chain, preferably a C1 alkyl chain;
have at least one, preferably to, more preferably all three of:
   (i) a reduced toxicity when cell viability is considered of cells contacted with the saponin derivatives;
   (ii) activity when potentiation of e.g. toxin cytotoxicity or BNA mediated gene silencing is considered (without wishing to be bound by any theory: relating to similar or improved endosomal escape enhancing activity of the modified saponin), if the aldehyde functional group is transformed to a hydrazone functional group according to formula (I); and/or
   (iii) reduced hemolytic activity,
when compared with the toxicity, activity and haemolytic activity of unmodified saponin on which the saponin derivative is based. Therewith, the inventors provide saponin derivatives with an improved therapeutic window, since for the saponin derivatives, the cytotoxicity is lower than cytotoxicity determined for their naturally occurring counterpart saponins, the haemolytic activity is lower than haemolytic activity determined for the naturally occurring counterparts of the saponin derivatives, the ratio between IC50 values for cell toxicity and e.g. IC50 values for toxin potentiation or IC50 value for gene silencing is similar or increased, and/or since the ratio between IC50 values for saponin haemolytic activity and e.g. IC50 value for toxin potentiation or IC50 value for gene silencing is similar or increased. Reference is made to the Examples section here below and to FIG. 9 and FIG. 10 for an overview of exemplified saponin derivatives, in combination with FIGS. 1-5, and to Table 3 and Table 4 for an overview of the cytotoxicity, haemolytic activity and endosomal escape enhancing activity (‘activity’), as well as an overview of the ratio between IC50 for cytotoxicity and IC50 for activity, and the ratio between IC50 for haemolytic activity and IC50 for activity, as determined on various (cancer) cells.

Surprisingly, transformation (derivatisation, modification) of the aldehyde group at C-23 of the aglycone of the saponin into a hydrazone functional group according to formula (I), results in a decrease in cytotoxicity when such saponin derivatives are contacted with cells, i.e. various types of cells. The decrease in cytotoxicity has been established by the inventors for the molecule with structure (VIII). It is thus part of the invention that the saponin derivatives of the invention with decreased cytotoxicity are provided, wherein the decrease in cytotoxicity is relative to the cytotoxicity as determined for the unmodified naturally occurring saponin counterparts. The saponin derivatives can be formed from such naturally occurring saponins, such as SO1861 and QS-21 (isoforms), preferably from SO1861. When the decrease in cytotoxicity is considered, saponin derivatives according to the invention, are equally suitable, when saponins with decreased cytotoxicity are to be provided. Furthermore, the inventors surprisingly established that tested modifications are suitable for lowering cytotoxicity, lowering haemolytic activity, and for preserving and remaining sufficiently extent of endosomal escape enhancing activity.

Furthermore, the inventors have found that saponin derivatives based on a saponin comprising a triterpene aglycone core structure and at least one of a first saccharide chain ‘R¹’ and a second saccharide chain ‘R²’ linked to the aglycone core structure, wherein the saponin derivative comprises an aglycone core structure comprising an aldehyde functional group which has been derivatised, wherein the aldehyde functional group is transformed to a hydrazone functional group according to formula (I)

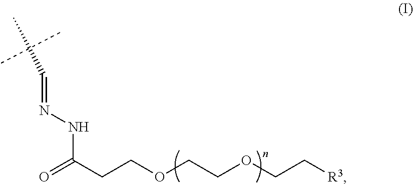

wherein n is an integer selected from 0-15,
R³ is azide or R³ is OH, or, preferably,
R³ is a cyclooctyne moiety selected from the group consisting of cyclooctyne moieties (II)a-d

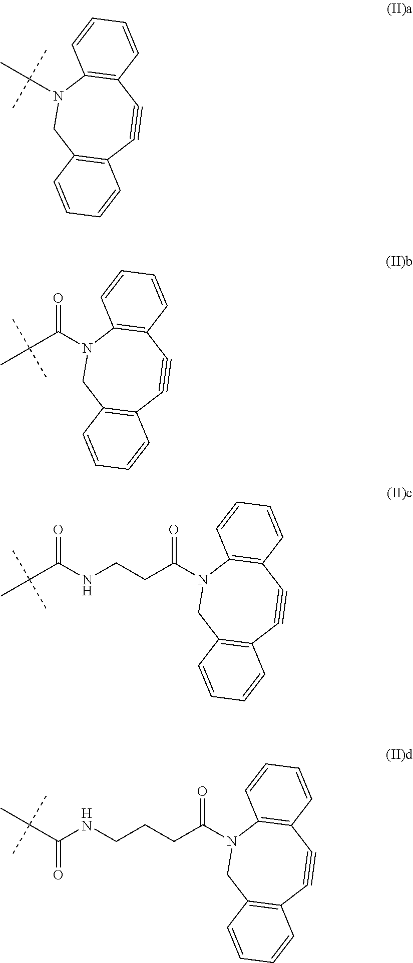

are valuable intermediate saponin derivatives towards the synthesis of yet further saponin derivatives according to the invention, preferably having at least one, more preferably at least two, most preferably all three of:
(i) having a reduced toxicity when cell viability is considered of cells contacted with the saponin derivatives;
(ii) have activity when potentiation of e.g. toxin cytotoxicity or BNA mediated gene silencing is considered (without wishing to be bound by any theory: relating to similar or improved endosomal escape enhancing activity of the modified saponin), if the aldehyde functional group is transformed to a hydrazone functional group according to formula (I); and/or
(iii) have reduced hemolytic activity,
when compared with the toxicity, activity and haemolytic activity of unmodified saponin. Furthermore, afore mentioned saponin derivatives obtained from derivatisation of saponin derivatives acting as intermediate saponin derivatives, are in itself also valuable intermediate saponin derivatives towards the synthesis of the saponin conjugate according to the invention (detailed below).

The inventors thus provide (intermediate) saponin derivatives with an improved therapeutic window when cytotoxicity is considered and/or when haemolytic activity is considered, and when the potentiation of e.g. toxins is considered compared to the corresponding underivatised saponin. As said, the saponin derivatives are end-products and are intermediates for the synthesis of yet further saponin derivatives. Such further saponin derivatives can act as intermediate saponin derivatives for the synthesis of saponin conjugates of the invention (see below). Such saponin derivatives of the invention are in particular suitable for application in a therapeutic regimen involving e.g. an ADC or an AOC for the prophylaxis or treatment of e.g. a cancer or an auto-immune disease such as rheumatoid arthritis. The safety of such saponin derivatives is improved when cytotoxicity and/or haemolytic activity is considered, especially when such saponin derivatives are administered to a patient in need of e.g. treatment with an ADC or with and AOC. Similar for the saponin derivatives formed from the saponin derivatives acting as intermediate saponin derivatives in the synthesis of such further saponin derivatives.

Thus, an aspect of the invention concerns a saponin derivative based on a saponin comprising a triterpene aglycone core structure and at least one of a first saccharide chain ‘R¹’ and a second saccharide chain ‘R²’ linked to the aglycone core structure, wherein the saponin derivative comprises an aglycone core structure comprising an aldehyde functional group which has been derivatised, wherein the aldehyde functional group is transformed to a hydrazone functional group according to formula (I)

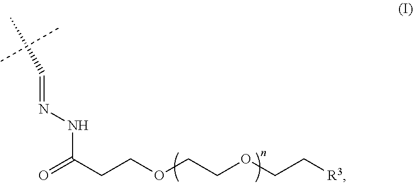

wherein n is an integer selected from 0-15,
R³ is azide or R³ is OH, or, preferably,
R³ is a cyclooctyne moiety selected from the group consisting of cyclooctyne moieties (II)a-d

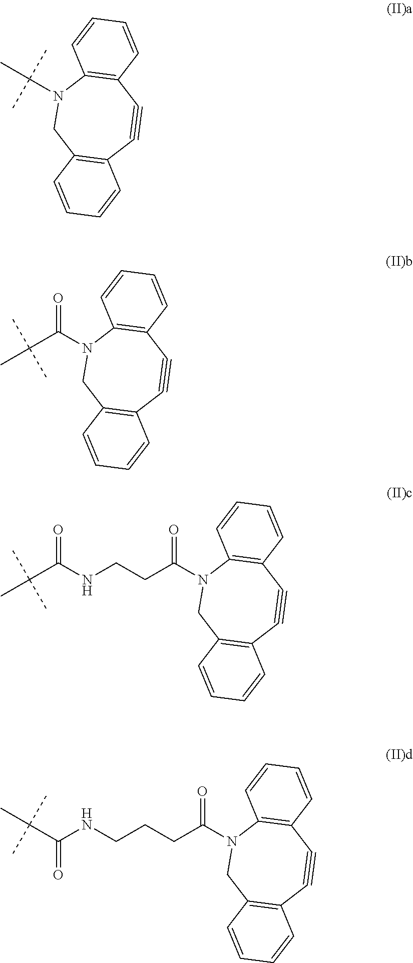

or,
R³ is a linker according to formula (III):

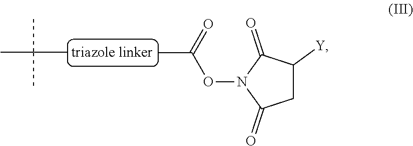

wherein Y is H or SO₂ONa, preferably H,
and the linker according to formula (III) is selected from the group consisting of linkers (IV)a-j:

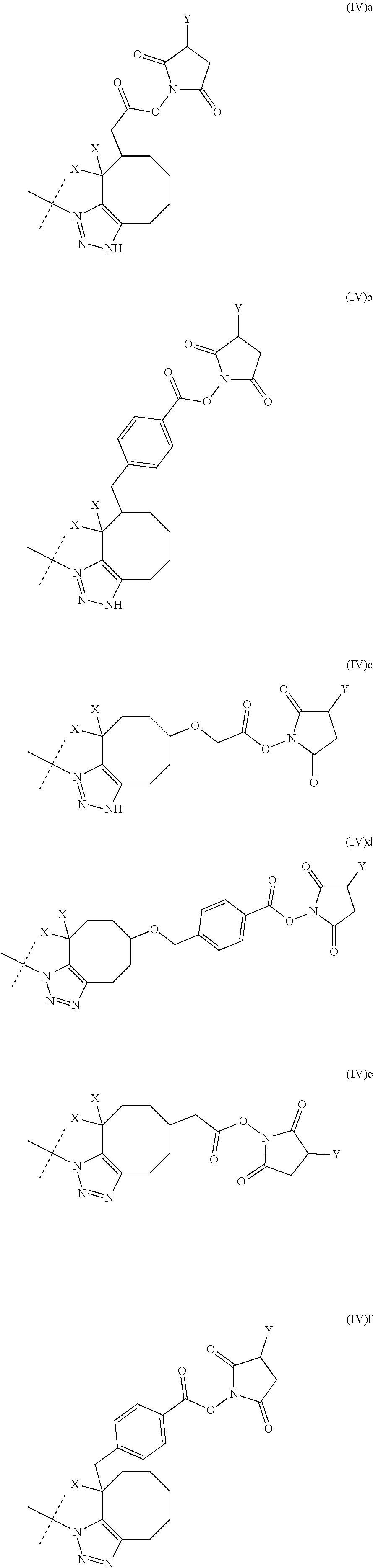

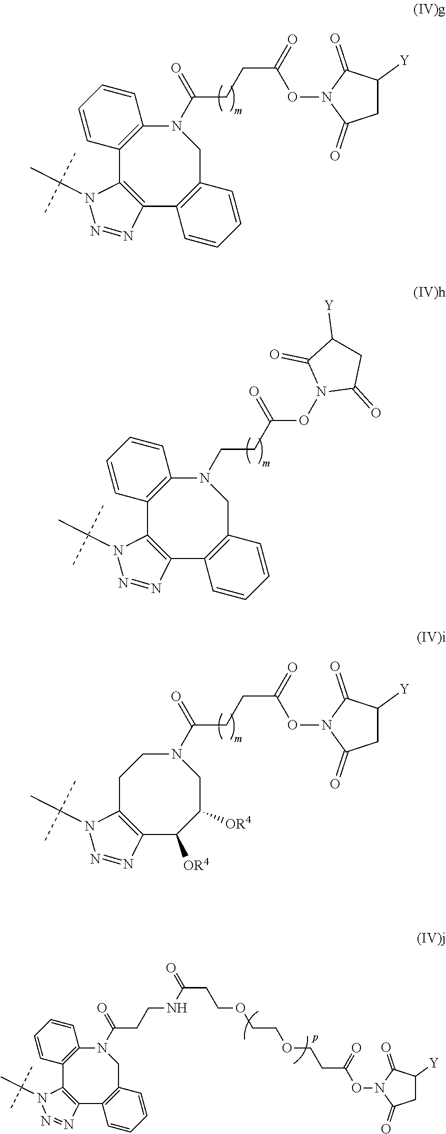

wherein X is H or F, preferably F;
m is 1 or 2;
p is an integer selected from 0-5, preferably 1→4, more preferably 2-3; and
R⁴ is a C1-C3 alkyl chain, preferably a C1 alkyl chain.

An embodiment of the invention is a saponin derivative, wherein the aldehyde functional group is transformed to a hydrazone functional group according to formula (I)

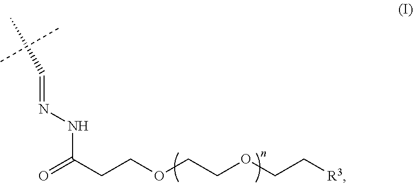

wherein n is an integer selected from 0-15,
R³ is azide or R³ is OH, or R³ is a cyclooctyne moiety, with the proviso that if the saponin derivative is an SO1861 derivative, n is not 3 if R³ is azide, and R³ is not azide if n is 3.

An embodiment of the invention is a saponin derivative, wherein the aldehyde functional group is transformed to a hydrazone functional group according to formula (I)

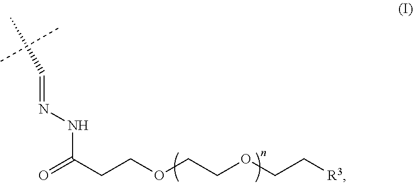

wherein n is an integer selected from 0-15,
R³ is azide or R³ is OH, or R³ is a cyclooctyne moiety, with the proviso that if the saponin derivative is an SO1861 derivative, said SO1861 derivative is not an SO1861 derivative with structure according to Formula (IV):

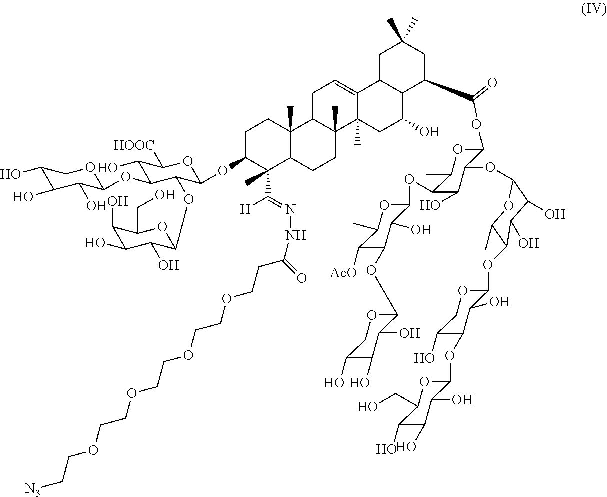

An aspect of the invention relates to a saponin derivative based on a saponin comprising a triterpene aglycone core structure and at least one of a first saccharide chain ‘R¹’ and a second saccharide chain ‘R²’ linked to the aglycone core structure, wherein the saponin derivative comprises an aglycone core structure comprising an aldehyde functional group which has been derivatised,
wherein the aldehyde functional group is transformed to a hydrazone functional group according to formula (A)

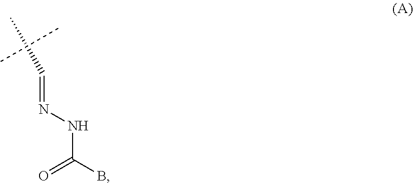

wherein B is H or B is a LINKER A, with the proviso that the saponin derivative is not the saponin derivative according to formula (AA1) and not the saponin derivative according to formula (AA2):

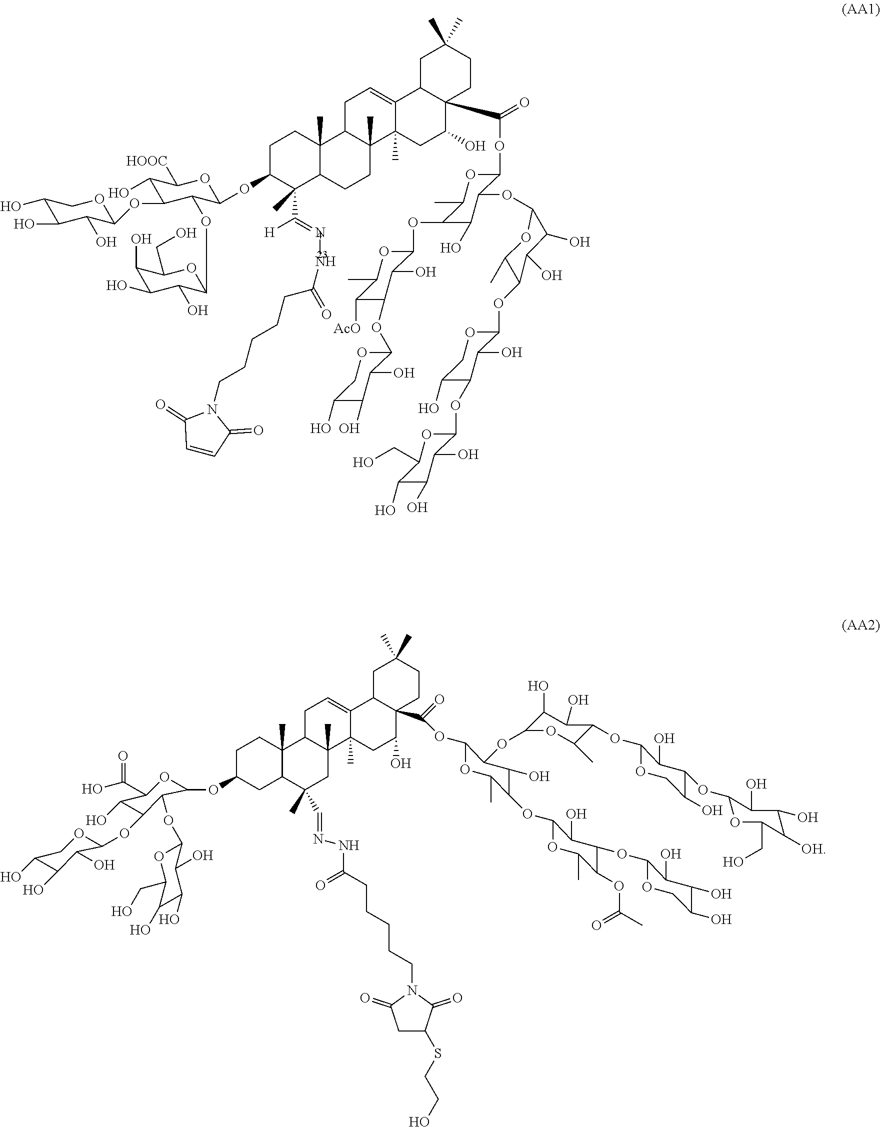

For example, for the saponin derivative wherein B is H in the hydrazone functional group according to formula (A), the hydrazone functional group is based on formic hydrazide. For example, for the saponin derivative wherein B is LINKER A in the hydrazone functional group according to formula (A), the LINKER A is a linker such as a linker selected from linkers commonly known in the art of bio-conjugation, with the proviso that the LINKER A is not the EMCH linker of molecule (AA1) when the saponin derivative is based on the saponin SO1861, and with the proviso that the LINKER A is not the EMCH-mercaptoethanol linker of molecule (AA2) when the saponin derivative is based on the saponin SO1861. An embodiment is the saponin derivative wherein B is LINKER A in the hydrazone functional group according to formula (A), wherein the LINKER A is a linker commonly known in the art of bio-conjugation, with the proviso that the LINKER A is not the EMCH linker of molecule (AA1) comprising SO1861, when the saponin derivative is based on the saponin SO1861, and with the proviso that the LINKER A is not the EMCH-mercaptoethanol linker of molecule (AA2) comprising SO1861, when the saponin derivative is based on the saponin SO1861, and/or with the proviso that the LINKER A is not the linker of molecule (AA3), which molecule (AA3) comprises linker EMCH-S-B1 and comprises SO1861, wherein B1 is a molecule based on a thiol (bearing molecule B1-SH such as a proteinaceous molecule comprising a cysteine residue) or based on a thiol-bearing polymeric structure,

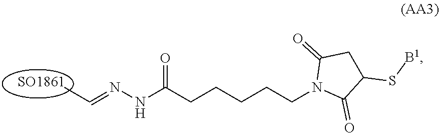

when the saponin derivative is based on the saponin SO1861.

An embodiment is the saponin derivative wherein B is LINKER A in the hydrazone functional group according to formula (A), wherein the LINKER A is a linker commonly known in the art of bio-conjugation, with the proviso that the LINKER A is not an acryl residue when the saponin on which the saponin derivative is based is SO1861, and/or with the proviso that the LINKER A is not an acrolyol residue when the saponin on which the saponin derivative is based is SO1861, and/or with the proviso that the LINKER A is not the EMCH linker of molecule (AA1) comprising SO1861, when the saponin derivative is based on the saponin SO1861, and with the proviso that the LINKER A is not the EMCH-mercaptoethanol linker of molecule (AA2) comprising SO1861, when the saponin derivative is based on the saponin SO1861, and/or with the proviso that the LINKER A is not the linker of molecule (AA3), which molecule (AA3) comprises linker EMCH-S-B1 and comprises SO1861, wherein B1 is a molecule based on a thiol (bearing molecule B1-SH such as a proteinaceous molecule comprising a cysteine residue) or based on a thiol-bearing polymeric structure,

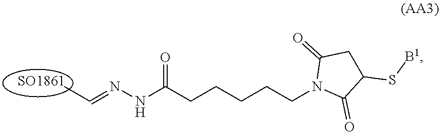

when the saponin derivative is based on the saponin SO1861.

Part of the invention is a the saponin derivative comprising an aglycone core structure comprising an aldehyde functional group which has been derivatised, wherein the aldehyde functional group is transformed to a hydrazone functional group according to according to formula (A):

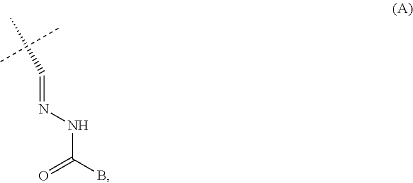

wherein B is the LINKER A wherein the LINKER A is according to the structure (AA4):

LINKER A-R³

(AA4),
thus comprising a functional group R³, wherein R³ is azide or R³ is OH, or R³ is a cyclooctyne moiety selected from the group consisting of cyclooctyne moieties (II)a-d

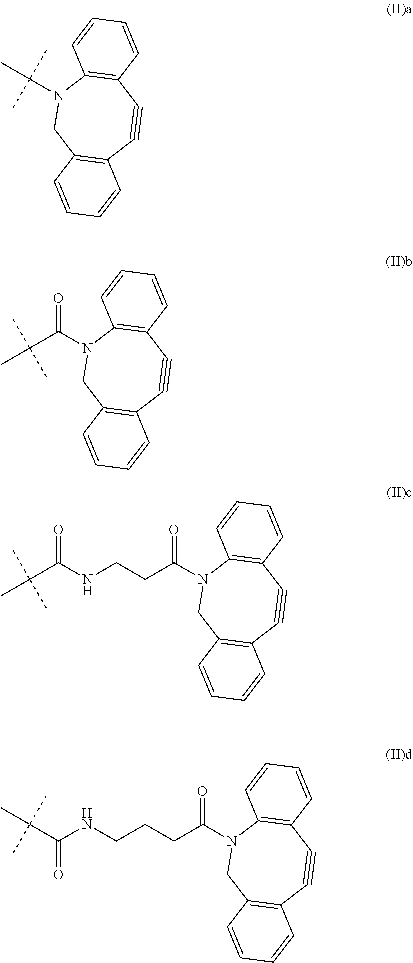

or,
R³ is a linker according to formula (III):

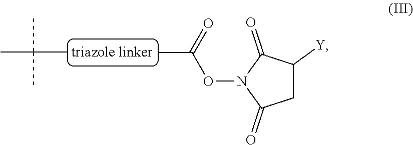

wherein Y is H or SO₂ONa, preferably H, and the linker according to formula (III) is selected from the group consisting of linkers (IV)a-j:

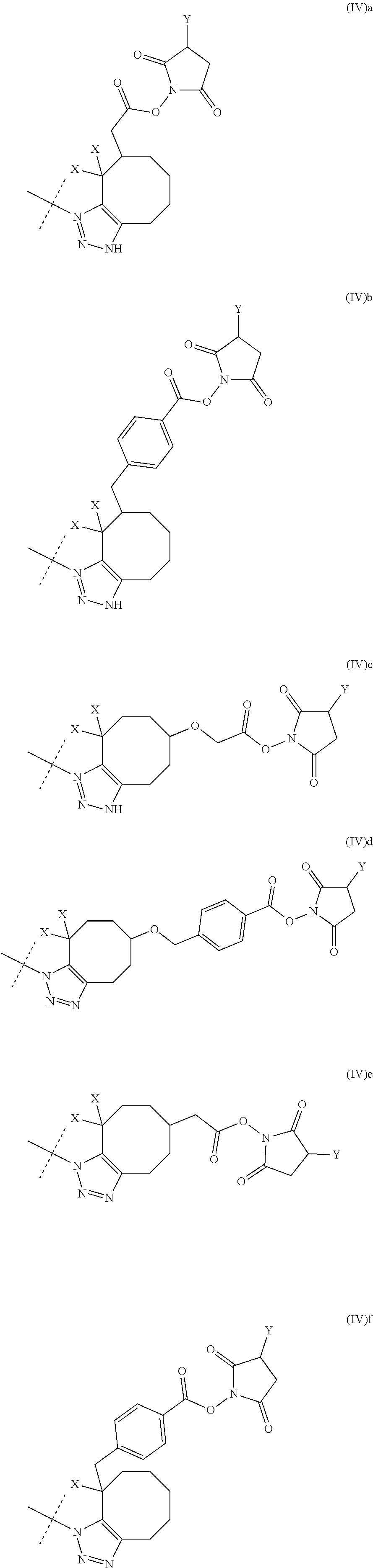

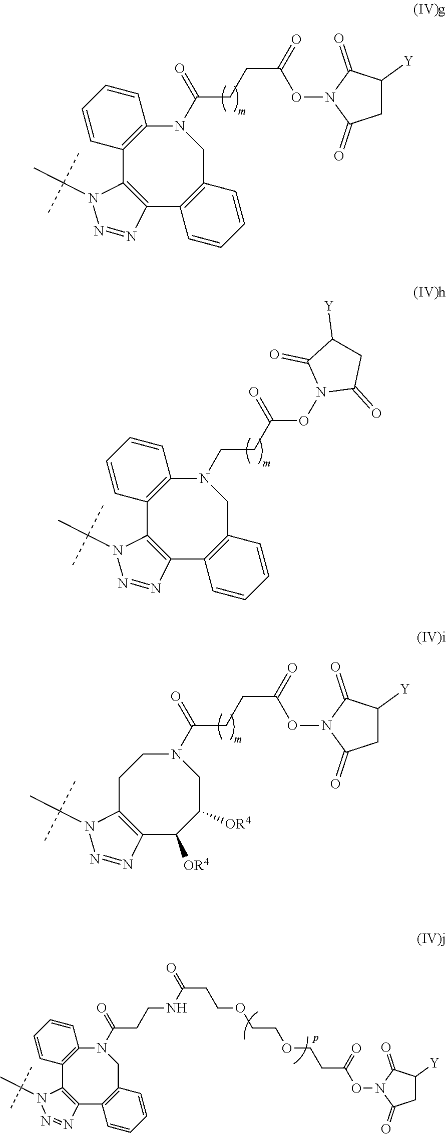

wherein X is H or F, preferably F;
m is 1 or 2;
p is an integer selected from 0-5, preferably 1→4, more preferably 2-3; and
R⁴ is a C1-C3 alkyl chain, preferably a C1 alkyl chain.

An aspect of the invention relates to saponin derivatives, wherein the aldehyde functional group is transformed to a hydrazone functional group according to formula (I)

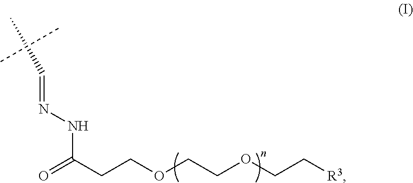

wherein n is an integer preferably selected from 0-15, and R³ is OH. Such saponin derivatives comprise a hydrazone functional group that is covalently linked to a polyethylene glycol (PEG) based on the structure HO—CH₂—CH₂—(O—CH₂—CH₂)ₙ—OH wherein n is an integer preferably selected from 0-15, more preferably 1-13 or 2-12 or 3-11 or 4-10. Such saponin derivative can thus have a selected number of PEG structural units or repeats —(O—CH₂—CH₂)ₙ— upon derivatisation of the aldehyde functional group of the aglycone of the saponin on which the derivative is based. Typically, such a saponin derivative comprises a PEG linker wherein n is an integer selected from 2-11, for example n is 3.

Worded differently, the saponin derivative according to the invention is a saponin comprising a triterpene aglycone core structure and at least one, preferably both, of a first saccharide chain ‘R¹’ and a second saccharide chain ‘R²’ linked to the aglycone core structure, wherein R¹ and R² are independently selected from hydrogen, a monosaccharide, a linear oligosaccharide and a branched oligosaccharide, preferably from a monosaccharide, a linear oligosaccharide and a branched oligosaccharide, and the aglycone core structure comprises an aldehyde group which has been derivatised by reacting with a hydrazide according to formula (3)

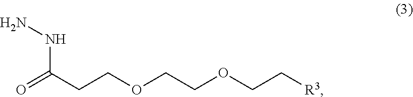

wherein n is an integer selected from 0-15;
R³ is azide or R³ is OH, or, preferably,
R³ is a cyclooctyne moiety selected from the group consisting of cyclooctyne moieties (II)a-d

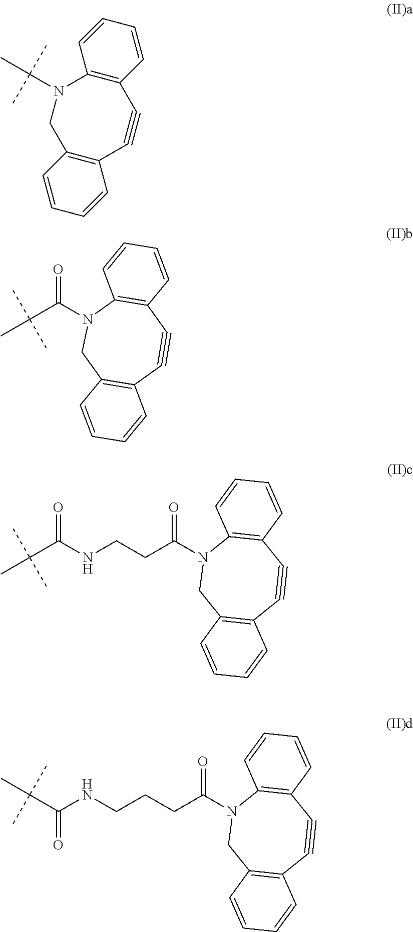

or,
R³ is a linker according to formula (III):

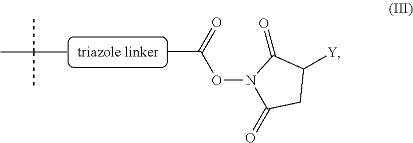

wherein Y is H or SO₂ONa, preferably H,
and the linker according to formula (III) is selected from the group consisting of linkers (IV)a-j:

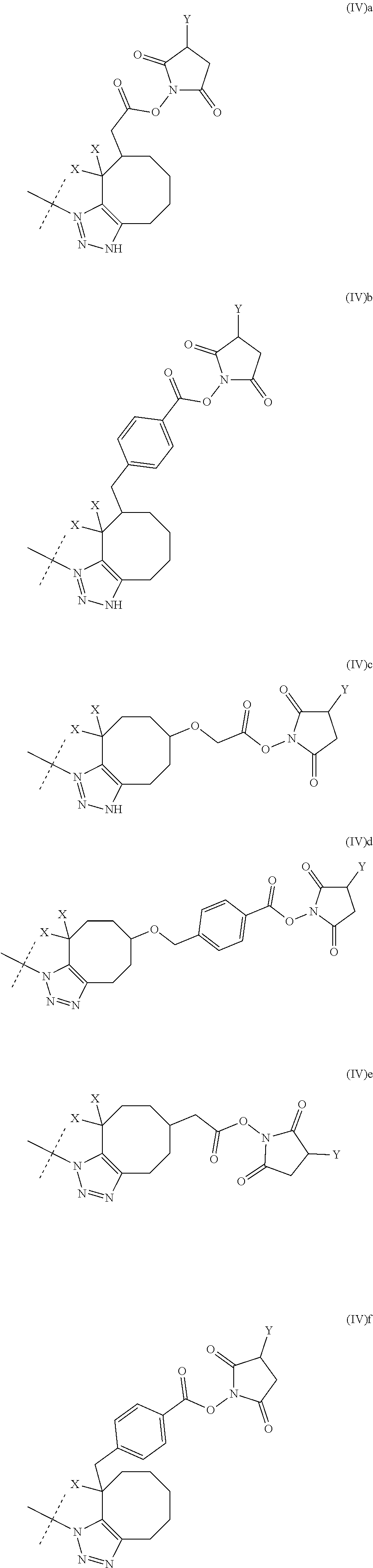

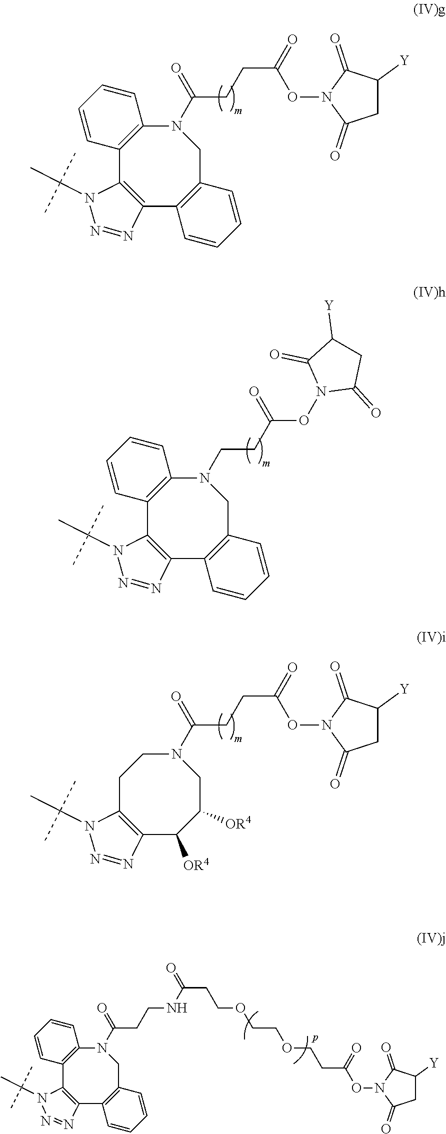

wherein X is H or F, preferably F;
m is 1 or 2, preferably 1;
p is an integer selected from 0-5, preferably 1→4, more preferably 2-3; and
R⁴ is a C1-C3 alkyl chain, preferably a C1 alkyl chain.

An embodiment is the saponin derivative according to the invention with the proviso that the saponin derivative is not according to formula (IV′)

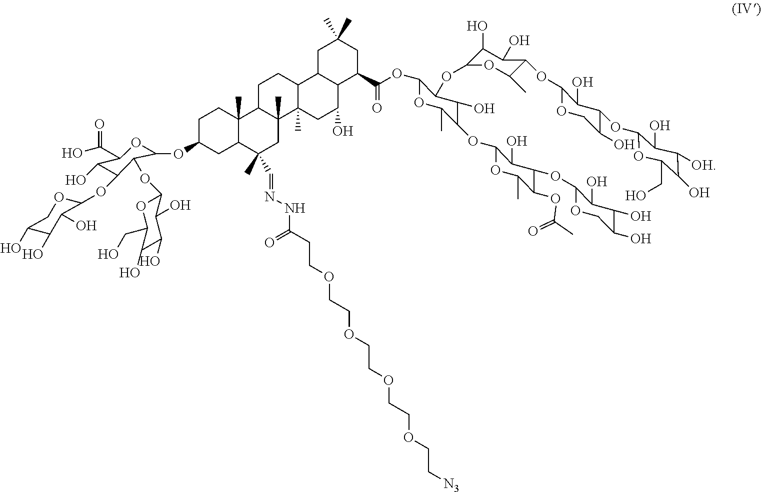

An embodiment is the saponin derivative according to the invention with the proviso that the saponin derivative is not according to formula (VIII)

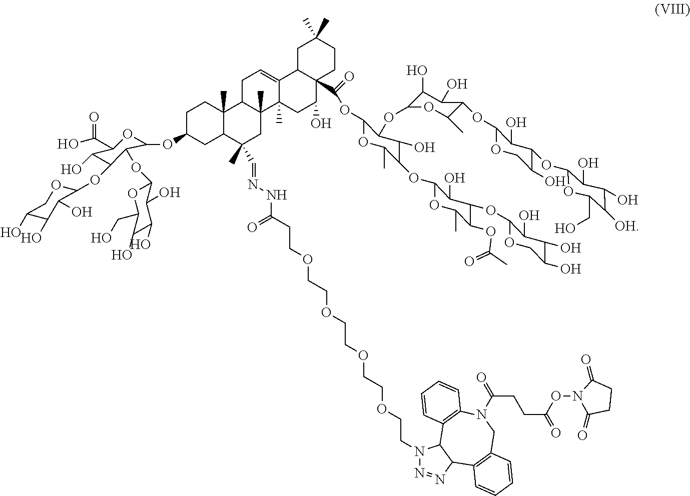

An embodiment is the saponin derivative according to the invention with the proviso that the saponin derivative is not according to formula (IV) and with the proviso that the saponin derivative is not according to formula (VIII).

Preferred is the saponin derivative according to the invention, wherein the saponin derivative is a monodesmosidic triterpene glycoside or a bidesmosidic triterpene glycoside, more preferably a bidesmosidic triterpene glycoside. A series of saponins known in the art have shown to enhance endosomal escape of molecules, which are most often bidesmosidic triterpene glycosides, although also a series of monodesmosidic triterpene glycosides have shown such enhancing activity. Table A1 summarizes the series of saponins for which endosomal escape enhancing activity has been established. Such saponins are good starting points for synthesising the saponin derivatives of the invention. In particular, SO1861 has proven to be a suitable natural saponin for derivatisation according to the invention.

Preferred is the saponin derivative according to the invention, wherein the saponin derivative comprises an aglycone core structure selected from the group consisting of:
2alpha-hydroxy oleanolic acid;
16alpha-hydroxy oleanolic acid;
hederagenin (23-hydroxy oleanolic acid);
16alpha, 23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtogenol C-21 (2-methylbut-2-enoate)-16 ,22-diacetate;
digitogenin;
3,16,28-trihydroxy oleanan-12-en;
gypsogenic acid,
and
derivatives thereof,
preferably the saponin derivative comprises an aglycone core structure selected from quillaic acid and gypsogenin or derivatives thereof, more preferably the saponin derivative aglycone core structure is quillaic acid or a derivative thereof. Since the inventors now found that improved saponin derivatives can be provided with regard to decreased cytotoxicity and lower haemolysis of cells contacted with such derivatives, while at the same time sufficient potentiating activity is conserved when potentiating the cytotoxic effect of e.g. an ADC is considered, based on saponins of the triterpene glycoside type, basically any saponin with such endosomal escape enhancing activity as tested by the inventors, such as saponins having the aglycone of the afore embodiment and listed in Table A1, can be improved accordingly. Lowering toxicity and lowering haemolytic activity while preserving activity to a sufficiently high extent when potentiation of toxins and for example BNAs is considered, is an important achievement by the inventors, when the widening of the therapeutic window of the saponin derivatives alone or in combination with e.g. an ADC or an AOC is considered. A sufficiently high dose of derivatised saponin can be applied in e.g. tumor therapy for a cancer patient in need thereof, while the (risk for) cytotoxic side-effects and the (risk for) undesired haemolytic activity exerted or induced by the saponin derivative is decreased when compared with the application of the natural saponin counterpart. Improvements of the therapeutic window of the saponin derivatives of the invention are for example apparent for the exemplified saponin derivatives in Table 3 and Table 4: the ratio between the 1050 for either cytotoxicity, or haemolytic activity and the 1050 for endosomal escape enhancing activity are listed, as well as the haemolytic activity, cytotoxicity and the activity.

An embodiment is the saponin derivative according to the invention, wherein the saponin derivative comprises an aglycone core structure selected from the group consisting of quillaic acid, gypsogenin, and derivatives thereof, preferably the saponin derivative comprises an aglycone core structure selected from the group consisting of quillaic acid and derivatives thereof, wherein the first saccharide chain R¹, when present, is linked to the C3 atom (also denoted as ‘C-3’ atom) or the C28 atom (also denoted as ‘C-28’ atom) of the aglycone core structure, preferably to the C3 atom, and/or wherein the second saccharide chain R², when present, is linked to the C28 atom of the aglycone core structure. Preferred are those saponin derivatives which are based on a saponin having both saccharide chains bound to the aglycone, but in general any saponin that displays endosomal escape enhancing activity is suitable for derivatisation according to the invention, for the purpose to provide derivatised saponins with lower cytotoxicity, and/or lower haemolytic activity and sufficiently high endosomal escape enhancing activity.

An embodiment is the saponin derivative according to the invention, wherein the saponin on which the saponin derivative is based is a penta-cyclic triterpene saponin of the 12,13-dehydrooleanane type, preferably the saponin is a monodesmosidic or bidesmosidic penta-cyclic triterpene saponin of the 12,13-dehydrooleanane type.

An embodiment is the saponin derivative according to the invention, wherein the saponin on which the saponin derivative is based is a mono-desmosidic or bi-desmosidic triterpene saponin belonging to the type of a 12,13-dehydrooleanane with the aldehyde group in position C-23 and optionally comprising a glucuronic acid group in a carbohydrate substituent at the C-3beta-OH group of the saponin, preferably a bi-desmosidic triterpene saponin belonging to the type of a 12,13-dehydrooleanane with the aldehyde group in position C-23 and comprising a glucuronic acid group in a carbohydrate substituent at the C-3beta-OH group of the saponin.

An embodiment is the saponin derivative according to the invention, wherein the saponin derivative is according to formula (V)

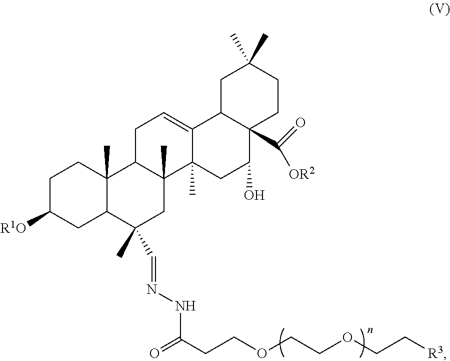

wherein R¹ and R² are independently selected from hydrogen, a monosaccharide, a linear oligosaccharide and a branched oligosaccharide;
n is an integer selected from 0-15;
R³ is azide; or
R³ is OH; or
R³ is a cyclooctyne moiety selected from the group consisting of cyclooctyne moieties (II)a-d

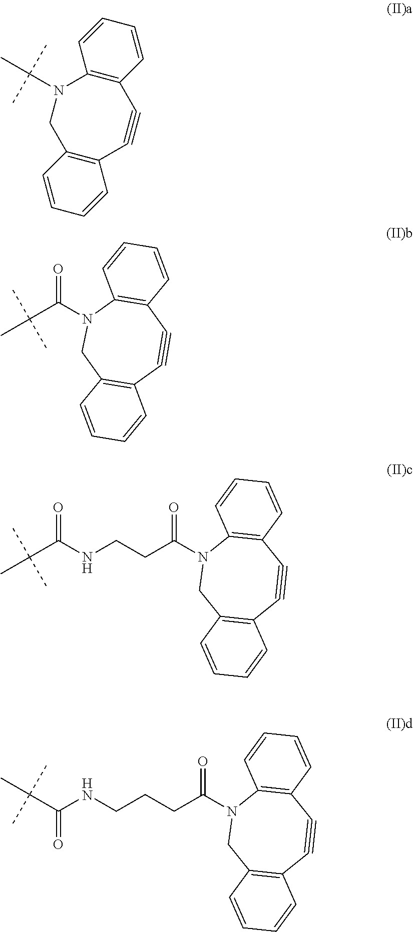

or;
R³ is a linker according to formula (III):

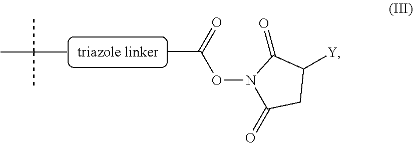

wherein Y is H or SO₂ONa, preferably H,
and the linker according to formula (III) is selected from the group consisting of linkers (IV)a-j:

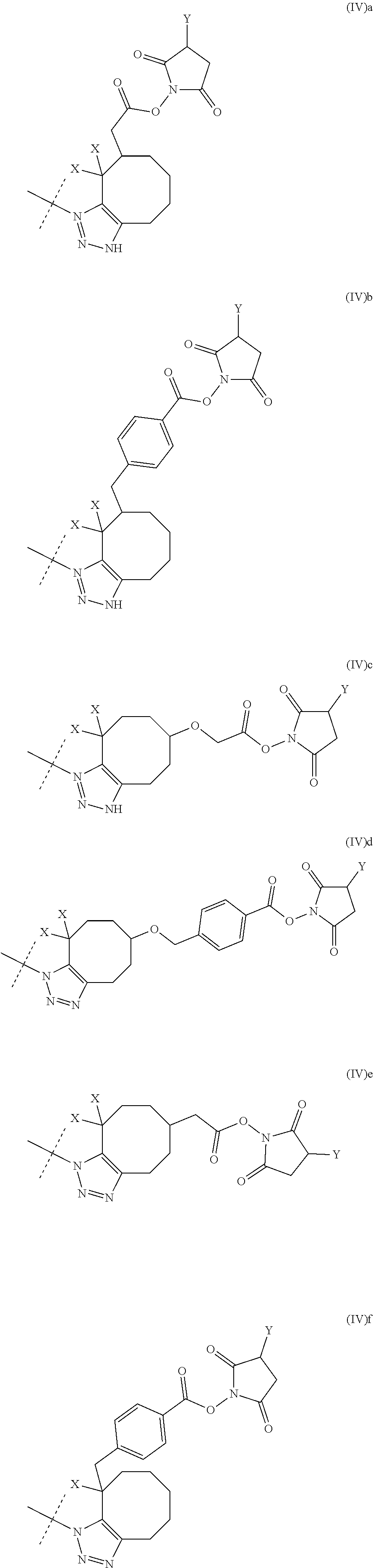

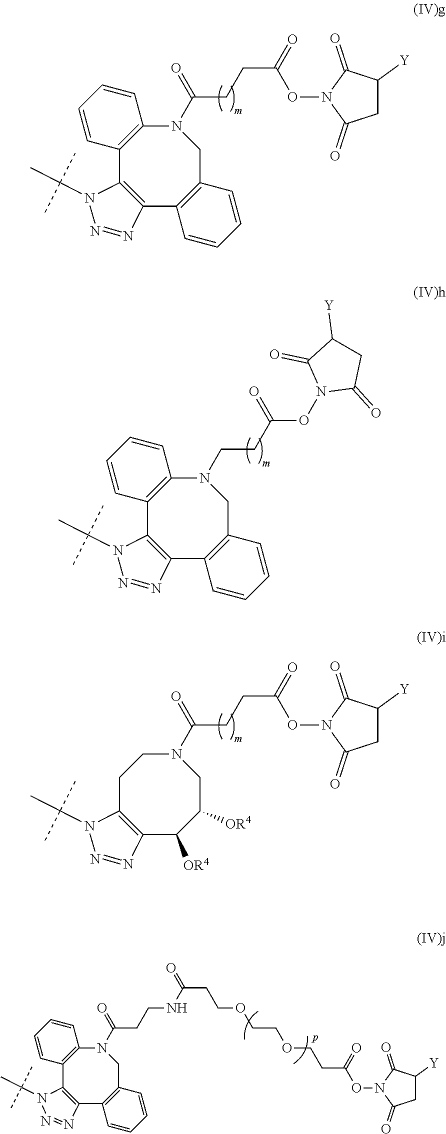

wherein X is H or F, preferably F;
m is 1 or 2, preferably 1;
p is an integer selected from 0-5, preferably 1→4, more preferably 2-3; and
R⁴ is a C1-C3 alkyl chain, preferably a C1 alkyl chain.

An embodiment is the saponin derivative according to the invention, wherein R¹ and R² are independently selected from hydrogen, a monosaccharide, a linear oligosaccharide and a branched oligosaccharide,
preferably R¹ is selected from
H,
GlcA-,
Glc-,
Gal-,
Rha-(1→2)-Ara-,
Gal-(1→2)-[Xyl-(1→3)]-GlcA-,
Glc-(1→2)-[Glc-(1→4)]-GlcA-,
Glc-(1→2)-Ara-(1→3)-[Gal-(1→2)]-GlcA-,
Xyl-(1→2)-Ara-(1→3)-[Gal-(1→2)]-GlcA-,
Glc-(1→3)-Gal-(1→2)-[Xyl-(1→3)]-Glc-(1→4)-Gal-,
Rha-(1→2)-Gal-(1→3)-[Glc-(1→2)]-GlcA-,
Ara-(1→4)-Rha-(1→2)-Glc-(1→2)-Rha-(1→2)-GlcA-,
Ara-(1→4)-Fuc-(1→2)-Glc-(1→2)-Rha-(1→2)-GlcA-,
Ara-(1→4)-Rha-(1→2)-Gal-(1→2)-Rha-(1→2)-GlcA-,
Ara-(1→4)-Fuc-(1→2)-Gal-(1→2)-Rha-(1→2)-GlcA-,
Ara-(1→4)-Rha-(1→2)-Glc-(1→2)-Fuc-(1→2)-GlcA-,
Ara-(1→4)-Fuc-(1→2)-Glc-(1→2)-Fuc-(1→2)-GlcA-,
Ara-(1→4)-Rha-(1→2)-Gal-(1→2)-Fuc-(1→2)-GlcA-,
Ara-(1→4)-Fuc-(1→2)-Gal-(1→2)-Fuc-(1→2)-GlcA-,
Xyl-(1→4)-Rha-(1→2)-Glc-(1→2)-Rha-(1→2)-GlcA-,
Xyl-(1→4)-Fuc-(1→2)-Glc-(1→2)-Rha-(1→2)-GlcA-,
Xyl-(1→4)-Rha-(1→2)-Gal-(1→2)-Rha-(1→2)-GlcA-,
Xyl-(1→4)-Fuc-(1→2)-Gal-(1→2)-Rha-(1→2)-GlcA-,
Xyl-(1→4)-Rha-(1→2)-Glc-(1→2)-Fuc-(1→2)-GlcA-,
Xyl-(1→4)-Fuc-(1→2)-Glc-(1→2)-Fuc-(1→2)-GlcA-,
Xyl-(1→4)-Rha-(1→2)-Gal-(1→2)-Fuc-(1→2)-GlcA-,
Xyl-(1→4)-Fuc-(1→2)-Gal-(1→2)-Fuc-(1→2)-GlcA-, and
derivatives thereof,
more preferably R¹ is Gal-(1→2)-[Xyl-(1→3)]-GlcA-; and
preferably R² is selected from:
H,
Glc-,
Gal-,
Rha-(1→2)-[Xyl-(1→4)]-Rha-,
Rha-(1→2)-[Ara-(1→3)-Xyl-(1→4)]-Rha-,
Ara-,
Xyl-,
Xyl-(1→4)-Rha-(1→2)-[R¹-(→4)]-Fuc- wherein R¹ is 4E-Methoxycinnamic acid,
Xyl-(1→4)-Rha-(1→2)-[R²-(→4)]-Fuc- wherein R² is 4Z-Methoxycinnamic acid,
Xyl-(1→4)-[Gal-(1→3)]-Rha-(1→2)-4-OAc-Fuc-,
Xyl-(1→4)-[Glc-(1→3)]-Rha-(1→2)-3,4-di-OAc-Fuc-,
Xyl-(1→4)-[Glc-(1→3)]-Rha-(1→2)-[R⁶-(→4)]-3-OAc-Fuc- wherein R⁶ is 4E-Methoxycinnamic acid,
Glc-(1→3)-Xyl-(1→4)-[Glc-(1→3)]-Rha-(1→2)-4-OAc-Fuc-,
Glc-(1→3)-Xyl-(1→4)-Rha-(1→2)-4-OAc-Fuc-,
(Ara- or Xyl-)(1→3)-(Ara- or Xyl-)(1→4)-(Rha- or Fuc-)(1→2)-[4-OAc-(Rha- or Fuc-)(1→4)]-(Rha-or Fuc-),
Xyl-(1→3)-Xyl-(1→4)-Rha-(1→2)-[Qui-(1→4)]-Fuc-,
Api-(1→3)-Xyl-(1→4)-[Glc-(1→3)]-Rha-(1→2)-Fuc-,
Xyl-(1→4)-[Gal-(1→3)]-Rha-(1→2)-Fuc-,
Xyl-(1→4)-[Glc-(1→3)]-Rha-(1→2)-Fuc-,
Ara/Xyl-(1→4)-Rha/Fuc-(1→4)-[Glc/Gal-(1→2)]-Fuc-,
Api-(1→3)-Xyl-(1→4)-[Glc-(1→3)]-Rha-(1→2)-[R⁷-(→4)]-Fuc- wherein R⁷ is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R⁸-(→4)]-Fuc- wherein R⁸ is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[Rha-(1→3)]-4-OAc-Fuc-,
Api-(1→3)-Xyl-(1→4)-[Glc-(1→3)]-Rha-(1→2)-[Rha-(1→3)]-4-OAc-Fuc-,
6-OAc-Glc-(1→3)-Xyl-(1→4)-Rha-(1→2)-[3-OAc-Rha-(1→3)]-Fuc-,
Glc-(1→3)-Xyl-(1→4)-Rha-(1→2)-[3-OAc--Rha-(1→3)]-Fuc-,
Xyl-(1→3)-Xyl-(1→4)-Rha-(1→2)-[Qui-(1→4)]-Fuc-,
Glc-(1→3)-[Xyl-(1→4)]-Rha-(1→2)-[Qui-(1→4)]-Fuc-,
Glc-(1→3)-Xyl-(1→4)-Rha-(1→2)-[Xyl-(1→3)-4-OAc-Qui-(1→4)]-Fuc-,
Xyl-(1→3)-Xyl-(1→4)-Rha-(1→2)-[3,4-di-OAc-Qui-(1→4)]-Fuc-,
Glc-(1→3)-[Xyl-(1→4)]-Rha-(1→2)-Fuc-,
6-OAc-Glc-(1→3)-[Xyl-(1→4)]-Rha-(1→2)-Fuc-,
Glc-(1→3)-[Xyl-(1→3)-Xyl-(1→4)]-Rha-(1→2)-Fuc-,
Xyl-(1→3)-Xyl-(1→4)-Rha-(1→2)-[Xyl-(1→3)-4-OAc-Qui-(1→4)]-Fuc-,
Api/Xyl-(1→3)-Xyl-(1→4)-[Glc-(1→3)]-Rha-(1→2)-[Rha-(1→3)]-40Ac-Fuc-,
Api-(1→3)-Xyl-(1→4)-[Glc-(1→3)]-Rha-(1→2)-[Rha-(1→3)]-40Ac-Fuc-,
Api/Xyl-(1→3)-Xyl-(1→4)-[Glc-(1→3)]-Rha-(1→2)-[R⁹-(→4)]-Fuc- wherein R⁹ is 5-O-[5-O-Rha-(1→2)-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R¹⁰-(→4)]-Fuc- wherein R¹⁰ is 5-O-[5-O-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R¹¹-(→4)]-Fuc- wherein R¹¹ is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R¹²-(→4)]-Fuc- wherein R¹² is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R¹³-(→4)]-Fuc- wherein R¹³ is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R¹⁴-(˜>3)]-Fuc- wherein R¹⁴ is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R¹⁵-(˜>3)]-Fuc- wherein R¹⁵ is 5-O-[5-O-Ara/Api-3,5-dihydroxy-6-methyl-octanoyl]-3,5-dihydroxy-6-methyl-octanoic acid)
Glc-(1→3)-[Glc-(1→6)]-Gal-, and
derivatives thereof;
more preferably R² is selected from:
Glc-(1→3)-Xyl-(1→4)-Rha-(1→2)-[Xyl-(1→3)-4-OAc-Qui-(1→4)]-Fuc-,
Xyl-(1→3)-Xyl-(1→4)-Rha-(1→2)-[3,4-di-OAc-Qui-(1→4)]-Fuc-,
Api-(1→3)-Xyl-(1→4)-Rha-(1→2)-[R¹²-(→4)]-Fuc- wherein R¹² is 5-O-[5-O-Ara/Api-3,5-dihydroxy-6-methyl-octanoyl]-3,5-dihydroxy-6-methyl-octanoic acid,
Xyl-(1→3)-Xyl-(1→4)-Rha-(1→2)-[R¹³-(→4)]-Fuc- wherein R¹³ is 5-O-[5-O-Ara/Api-3,5-dihydroxy-6-methyl-octanoyl]-3,5-dihydroxy-6-methyl-octanoic acid,
Api-(1→3)-Xyl-(1→4)-Rha-(1→2)-[R¹⁴-(˜>3)]-Fuc- wherein R¹⁴ is 5-O-[5-O-Ara/Api-3,5-dihydroxy-6-methyl-octanoyl]-3,5-dihydroxy-6-methyl-octanoic acid, and
Xyl-(1→3)-Xyl-(1→4)-Rha-(1→2)-[R¹⁵-(˜>3)]-Fuc- wherein R¹⁵ is 5-O-[5-O-Ara/Api-3,5-dihydroxy-6-methyl-octanoyl]-3,5-dihydroxy-6-methyl-octanoic acid,
most preferably R¹ is Gal-(1→2)-[Xyl-(1→3)]-GlcA- and R² is Glc-(1→3)-Xyl-(1→4)-Rha-(1→2)-[Xyl-(1→3)-4-OAc-Qui-(1→4)]-Fuc-.

Preferred is the saponin derivative according to the invention, wherein the saponin derivative is a derivative of a saponin selected from the group of saponins consisting of: Quillaja bark saponin, dipsacoside B, saikosaponin A, saikosaponin D, macranthoidin A, esculentoside A, phytolaccagenin, aescinate, AS6.2, NP-005236, AMA-1, AMR, alpha-Hederin, NP-012672, NP-017777, NP-017778, NP-017774, NP-018110, NP-017772, NP-018109, NP-017888, NP-017889, NP-018108, SA1641, AE X55, NP-017674, NP-017810, AG1, NP-003881, NP-017676, NP-017677, NP-017706, NP-017705, NP-017773, NP-017775, SA1657, AG2, SO1861, GE1741, SO1542, SO1584, SO1658, SO1674, SO1832, SO1904, SO1862, QS-7, QS1861, QS-7 api, QS1862, QS-17, QS-18, QS-21 A-apio, QS-21 A-xylo, QS-21 B-apio, QS-21 B-xylo, beta-Aescin, Aescin la, Teaseed saponin I, Teaseedsaponin J, Assamsaponin F, Digitonin, Primula acid 1 and AS64R, preferably the saponin derivative is selected from the group consisting of a QS-21 derivative, an SO1861 derivative, an SA1641 derivative and an GE1741 derivative, more preferably the saponin derivative is selected from the group consisting of a QS-21 derivative and an SO1861 derivative, most preferably the saponin derivative is an SO1861 derivative. These saponins are essentially saponins displaying endosomal escape enhancing activity as established by the inventors or by others, or that are structurally highly similar to saponins for which the endosomal escape enhancing activity has been established. Structural outline of these saponins is summarized in Table A1.

Also preferred is the saponin derivative according to the invention, wherein the saponin derivative is selected from the group consisting of derivatives of: SO1861, SA1657, GE1741, SA1641, QS-21, QS-21A, QS-21 A-api, QS-21 A-xyl, QS-21B, QS-21 B-api, QS-21 B-xyl, QS-7-xyl, QS-7-api, QS-17-api, QS-17-xyl, QS1861, QS1862, Quillajasaponin, Saponinum album, QS-18, Quil-A, Gyp1, gypsoside A, AG1, AG2, SO1542, SO1584, SO1658, SO1674, SO1832, SO1862, SO1904, stereoisomers thereof and combinations thereof, preferably the saponin derivative is selected from the group consisting of an SO1861 derivative, a GE1741 derivative, an SA1641 derivative, a QS-21 derivative, and a combination thereof, more preferably the saponin derivative is an SO1861 derivative or a QS21 derivative, most preferably, the saponin derivative is an SO1861 derivative.

A preferred embodiment is the saponin derivative according to the invention, wherein the saponin derivative is according to formula (VI)

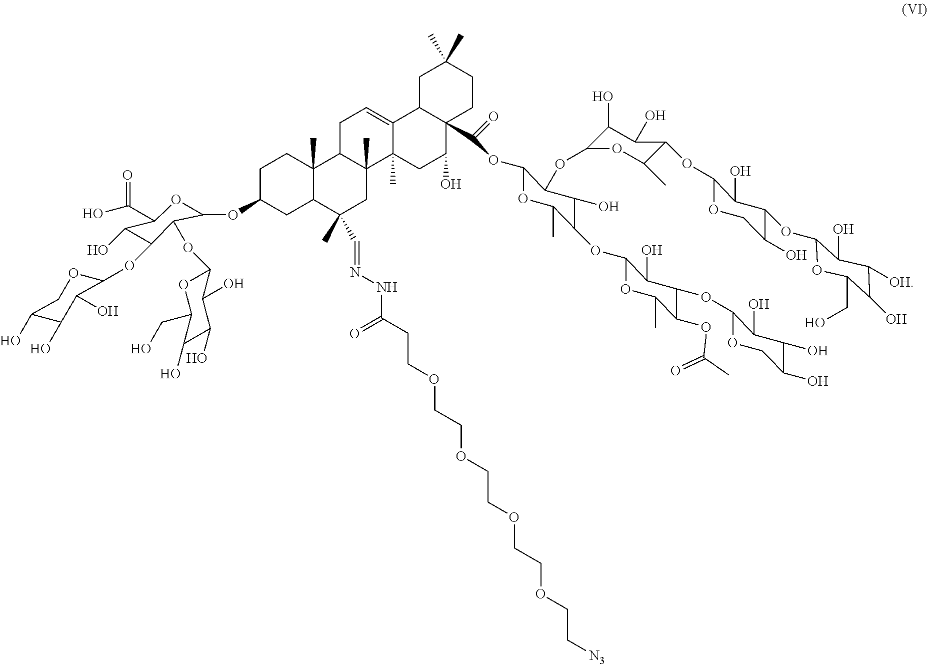

Such a saponin derivate according to molecule (VI) is particularly suitable for coupling to, for example, a ligand for binding to a cell-surface molecule (e.g. via a linker), such as an antibody for binding to a cell-surface receptor. This way, the molecule (VI) serves as an intermediate saponin derivative product for involvement in the synthesis of a saponin-antibody conjugate. In addition, molecule (VI) is a suitable saponin derivative for coupling to e.g. DBCO, providing yet a further saponin derivative of the invention. Again, such 501861-PEG4-DBCO is a saponin derivative of the invention, and is yet an intermediate saponin derivative for subsequently forming a saponin derivate comprising e.g. NHS bound to the DBCO. Herewith, molecule (VI) is a suitable pre-cursor saponin derivative for synthesizing further saponin derivatives of the invention such as a derivative based on the linker of formula (III), all comprising the hydrazone bond between the saponin and the linker PEG4, hydrolysable at slightly acidic pH (about 4,5-6,5, preferably 4,5-5,5), as apparent in the endosome of e.g. mammalian cells such as human tumor cells, said further saponin derivatives in turn being suitable for synthesising e.g. saponin-antibody conjugates, which also comprise the beneficial feature of the hydrazone bond between the saponin and the linker PEG4, hydrolysable at slightly acidic pH (about 4,5-6,5, preferably 4,5-5,5), therewith facilitating transfer of the saponin over the cell membrane into the endosome, and subsequent release from the linker in the endosome (suitable pH apparent), such that the saponin can exert its endosomal escape enhancing activity towards any (selected, desired) co-molecule delivered or present in the endosome and aimed for delivery into the cytosol of the cell.

An embodiment is the saponin derivative according to the invention, wherein the saponin derivative is according to formula (VII)

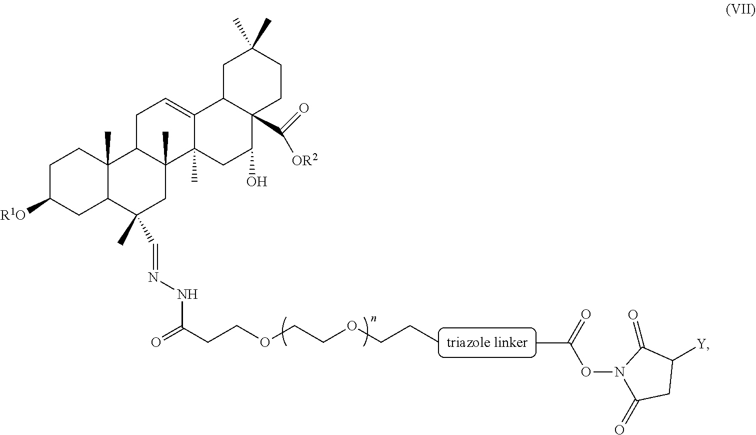

wherein R¹ and R² are independently selected from hydrogen, a monosaccharide, a linear oligosaccharide and a branched oligosaccharide,
n is an integer selected from 0-14,
Y=H or SO₂ONa, preferably H,
the linker

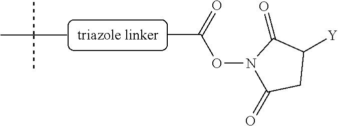

(formula (III)) is selected from the group consisting of linkers (IV)a-j:

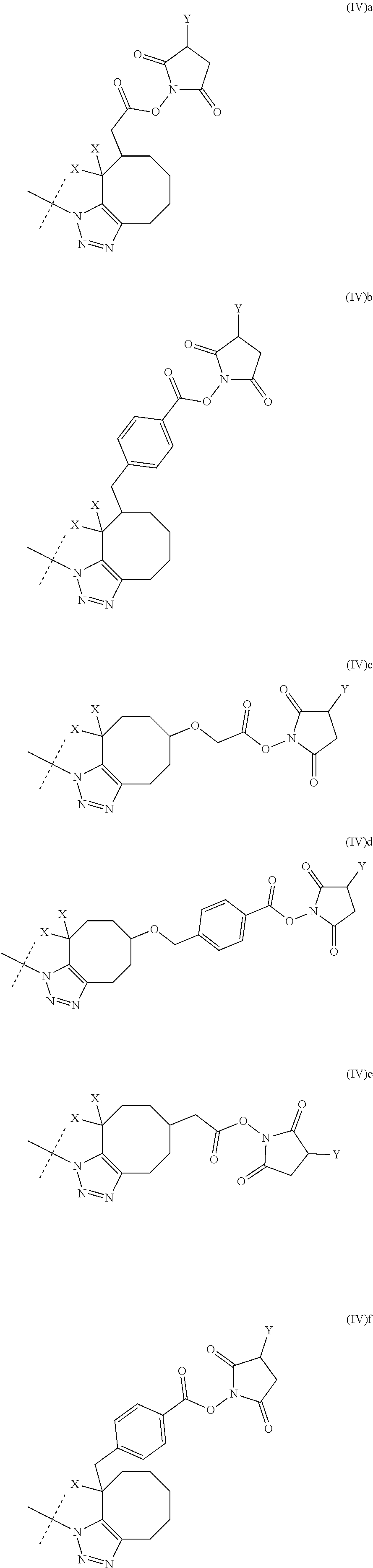

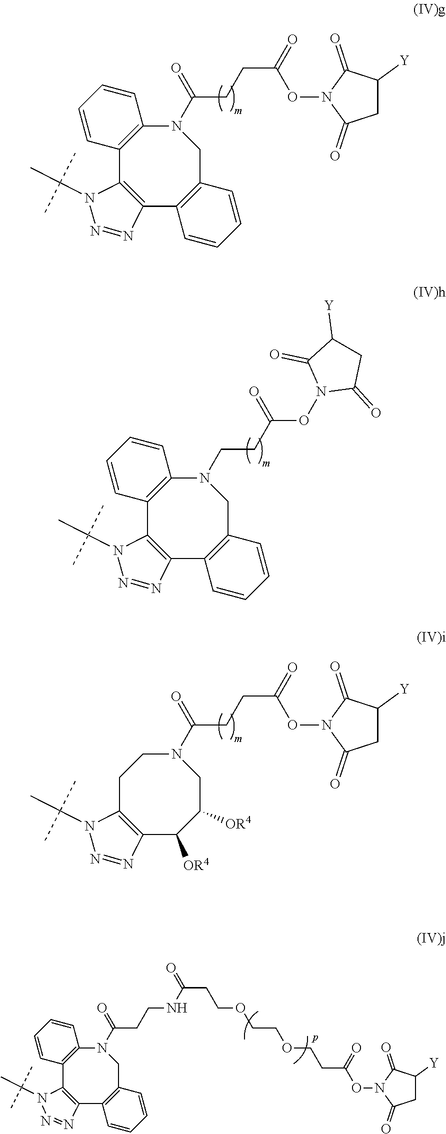

wherein X=H or F, preferably F;
m is 1 or 2, preferably 1;
p is an integer selected from 0-5, preferably 1→4, more preferably 2-3; and
R⁴ is a C1-C3 alkyl chain, preferably a C1 alkyl chain.

Preferred is the saponin derivative according to the invention, wherein the hydrazone functional group according to formula (I), the saponin derivative according to formula (V) or the saponin derivative according to formula (VII), wherein n is an integer selected from 1-12, preferably 2-9, more preferably 3-6. Particularly preferred is the saponin derivative according to the invention, wherein the hydrazone functional group according to formula (I) or the saponin derivative according to formula (V), wherein n is 3, although PEG linkers with shorter or longer length are also suitable for provision of saponin derivatives comprising the hydrazone bond between the saponin aglycone core and the bound linker. One of the several benefits of the saponin derivatives of the invention is the high degree of flexibility in design of the saponin derivative when the length of the PEG linker is considered and when the structural and chemical nature of the linker with formula (III) is considered, as long as the PEG linker and subsequently linked further chemical moieties and groups are linked to the saponin through the acid-hydrolysable hydrazone bond. The advantage of the hydrazone bond is the capacity to become hydrolysed under the slightly acidic conditions as apparent in the endosome of mammalian cells such as human cells such as tumor cells. Upon hydrolyzing the hydrazone bond, the PEG linker including any further linkers and moieties, when present, splits of from the saponin, and the aldehyde functional group is formed on the saponin aglycone, as present on the ‘wild type’ saponin that was selected for initial synthesis of the saponin derivative. Without wishing to be bound by any theory, it is assumed that re-appearance of the aldehyde functional group contributes to the endosomal escape enhancing activity of the saponin derivative, once the saponin derivative entered the endosome, while at the same time ‘shielding’ of the aldehyde functional group by forming the hydrazone bond in de saponin derivative, during transfer of the saponin derivative from the extracellular space into the endosome, aids in reducing the occurrence or risk for cytotoxic effects and/or (excessive) hemolytic activity.

A preferred embodiment is the saponin derivative according to the invention, wherein the linker

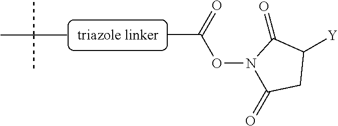

according to formula (III) is selected from the group consisting of linkers (IV)g-(IV)j, preferably the linker is linker (IV)g or (IV)h, more preferably the linker is linker (IV)g.

Preferred is the saponin derivative according to the invention, wherein the saponin derivative is a compound according to formula (VIII)

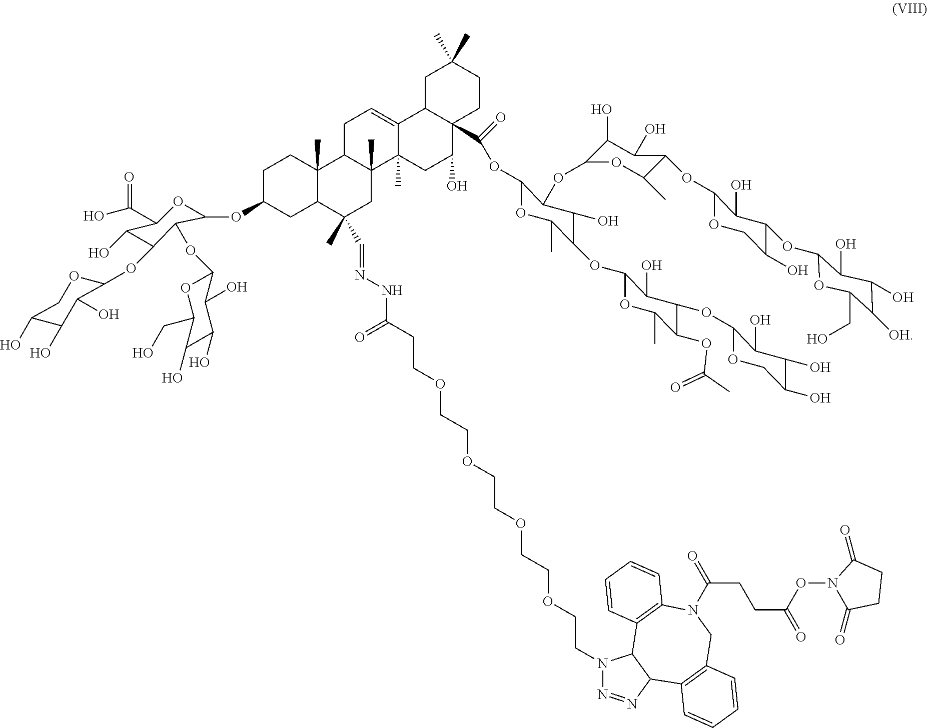

Also preferred is the saponin derivative wherein the saponin is selected from the saponins listed in Table A1 and wherein the PEG linker comprises 1-15 repeats in the chain (n is an integer selected from 0-14) and wherein the PEG linker is further linked to the molecule (III) with formula (IV)g with m=1 and Y is H. In particular, the PEG linker is a PEG4 linker (thus, n is 3) and the saponin is any of QS21, QS7, SA1641, GE1741 and SO1861, preferably QS21 or SO1861, more preferably SO1861, and the molecule (III) has the structure of formula (IV)g with m=1 and Y is H. The PEG linker may also be any of a PEG1-PEG16 linker, and PEG4 is preferred.

Preferably, the saponin derivative according to the invention is characterized in that the saponin derivative comprises a single saponin moiety, although saponin derivatives which comprise multiple saponin moieties each coupled to a common molecule through hydrazone bonds are equally preferred. Herein, the saponins from which such saponin derivatives are construed are saponins comprising an aldehyde functional group in their aglycone, said aldehyde group transformed into the hydrazone bond (functional group) when reacted with a hydrazine functional group.

Also preferred is the saponin derivative according to the invention, characterized in that the saponin derivative has a molecular weight of less than 2500 g/mol, preferably less than 2300 g/mol, more preferably less than 2150 g/mol. Typically, such saponin derivatives are based on the saponins listed in Table A1 . Typically, such saponin derivatives comprise a PEG linker consisting of 2-10 building blocks. Typically, such saponin derivative comprises DBCO-NHS or DBCO linked to the PEG linker.

Preferred is the saponin derivative according to the invention, characterized in that the saponin derivatisation has a molecular weight of less than 400 g/mol, preferably less than 300 g/mol, more preferably less than 270 g/mol. The molecular weight of the saponin derivatisation corresponds to the molecular weight of the saponin derivative exclusive of the aglycone core and the one (for monodesmosidic saponins) or two (for bidesmosidic saponins) glycon (sugar) chains.

Pharmaceutical Compositions and Combinations Comprising a Saponin Derivative

An aspect of the invention relates to a first pharmaceutical composition comprising the saponin derivative according to the invention and optionally a pharmaceutically acceptable excipient and/or diluent.

Preferred is the first pharmaceutical composition according to the invention comprising the saponin derivative according to the invention, and preferably a pharmaceutically acceptable diluent, and further comprising:
a pharmaceutically acceptable salt, preferably a pharmaceutically acceptable inorganic salt, such as an ammonium, calcium, copper, iron, magnesium, manganese, potassium, sodium, strontium or zinc salt, preferably NaCl; and/or
a pharmaceutically acceptable buffer system, such as a phosphate, a borate, a citrate, a carbonate, a histidine, a lactate, a tromethamine, a gluconate, an aspartate, a glutamate, a tartarate, a succinate, a malate, a fumarate, an acetate and/or a ketoglutarate containing buffer system.

Preferred is the first pharmaceutical composition according to the invention comprising the saponin derivative according to the invention and a pharmaceutically acceptable diluent, preferably water, wherein the composition is liquid at a temperature of 25° C., and has a pH within the range of 2-11, preferably within the range of 4-9, more preferably within the range of 6-8.

Preferred is the first pharmaceutical composition according to the invention comprising a saponin derivative according to the invention and a pharmaceutically acceptable diluent, preferably water, wherein the composition is liquid at a temperature of 25° C. and wherein the concentration of the saponin derivative is within the range of 10-12 to 1 mol/I, preferably within the range of 10-9 to 0.1 mol/I, more preferably within the range of 10-6 to 0.1 mol/I.

Typically, such a first pharmaceutical composition is suitable for use in combination with e.g. an ADC or an AOC. For example, the first pharmaceutical composition is administered to a patient in need of administration of the ADC or AOC, before the ADC or AOC is administered, together with the ADC or AOC, or (shortly, e.g. within 1 second-30 minutes) after administration of the ADC or the AOC to the patient in need of such ADC or AOC therapy. For example, the first pharmaceutical composition is mixed with a pharmaceutical composition comprising the ADC or the AOC, and a suitable dose of the mixture obtained is administered to a patient in need of ADC or AOC therapy. According to the invention, the saponin derivative comprised by the first pharmaceutical composition enhances the efficacy and potency of the effector molecule, such as a protein toxin or a BNA or siRNA, comprised by such an ADC or AOC, when the saponin derivative and the ADC or AOC co-localize inside a target cell such as a tumor cell. Under influence of the saponin derivative, the effector molecule is released into the cytosol of the target cell to a higher extent, compared to contacting the same cells with the same dose of ADC or AOC in the absence of the saponin derivative. Thus, similar efficacy can be obtained at lower ADC or AOC dose when the effector molecule co-localizes inside a target cell together with the saponin derivative of the first pharmaceutical composition, compared to the dose required to achieve the same efficacy in the absence of the saponin derivative inside the cell where the ADC or the AOC comprising the effector molecule is delivered. Typically, saponin derivatives wherein R³ is OH are suitable derivatives for application in the first pharmaceutical composition of the invention. The aldehyde functional group of the original saponin is transformed into a hydrazone functional group in the saponin derivative, and a PEG is coupled to the hydrazone functional group, such that under slightly acidic conditions as apparent in the endosome of mammalian cells, the hydrazone functional group together with the PEG split off from the aglycone under formation of the original aldehyde functional group, therewith providing the saponin on which the saponin derivate was based. Inside the endosome, the formed saponin can then exert its endosomal escape enhancing activity towards any effector molecule or effector moiety (originally) comprised by e.g. an ADC or AOC, that is co-localized in the endosome, resulting in said effector molecule or effector moiety entering the cell cytosol and, dependent on the type of molecule or moiety, ultimately in the nucleus.

Preferred is the first pharmaceutical composition of the invention, wherein the saponin derivative is the saponin derivative according to formula (VIII). Such a saponin derivative is efficacious in enhancing the potency of e.g. an ADC or AOC (measured as extent of cell killing) when cells are contacted with a mixture of the ADC or AOC and the saponin derivative.

An aspect of the invention relates to a first pharmaceutical combination comprising:
the first pharmaceutical composition of the invention; and
a second pharmaceutical composition comprising any one or more of: a conjugate of a cell-surface molecule binding-molecule and an effector moiety, such as an antibody-effector moiety conjugate, a receptor-ligand—effector moiety conjugate, an antibody-toxin conjugate, a receptor-ligand—toxin conjugate, an antibody-drug conjugate, a receptor-ligand—drug conjugate, an antibody-oligonucleotide conjugate and a receptor-ligand—oligonucleotide conjugate, and optionally comprising a pharmaceutically acceptable excipient and/or diluent.

An aspect of the invention relates to a pharmaceutical combination comprising:
the first pharmaceutical composition of the invention; and
a second pharmaceutical composition comprising any one or more of an antibody-effector moiety conjugate, a receptor-ligand—effector moiety conjugate, an antibody-toxin conjugate, a receptor-ligand—toxin conjugate, an antibody-drug conjugate, a receptor-ligand—drug conjugate, an antibody-oligonucleotide conjugate or a receptor-ligand—oligonucleotide conjugate, and optionally comprising a pharmaceutically acceptable excipient and/or diluent.

An aspect of the invention relates to a third pharmaceutical composition comprising the saponin derivative of the invention and further comprising any one or more of: a conjugate of a cell-surface molecule binding-molecule and an effector moiety, an antibody-effector moiety conjugate, a receptor-ligand—effector moiety conjugate, an antibody-toxin conjugate, a receptor-ligand—toxin conjugate, an antibody-drug conjugate, a receptor-ligand—drug conjugate, an antibody-nucleic acid conjugate or a receptor-ligand—nucleic acid conjugate, and optionally comprising a pharmaceutically acceptable excipient and/or diluent.

An aspect of the invention relates to a third pharmaceutical composition comprising the saponin derivative of the invention and further comprising any one or more of: an antibody-effector moiety conjugate, a receptor-ligand—effector moiety conjugate, an antibody-toxin conjugate, a receptor-ligand—toxin conjugate, an antibody-drug conjugate, a receptor-ligand—drug conjugate, an antibody-nucleic acid conjugate or a receptor-ligand—nucleic acid conjugate, and optionally comprising a pharmaceutically acceptable excipient and/or diluent.

Such a receptor ligand can be a receptor ligand known in the art of cell-targeting therapy, such as EGF. Typically, such a receptor ligand targets a receptor on a diseased cell such as a tumor cell or an auto-immune cell such as in rheumatoid arthritis. Typically, such a receptor ligand is a proteinaceous molecule such as a peptide or a protein, although non-proteinaceous receptor ligands known in the art are equally suitable. Such receptor ligands can also be selected from molecules such as adnectins, anticalins, affibodies, etc., etc. The common denominator for the receptor ligands is their specificity for an epitope on a target cell, for targeted delivery of an effector moiety bound to the receptor ligand, reminiscent to the targeted delivery of effector moieties such as toxins, enzymes, oligonucleotides, drug molecules, etc., linked to e.g. an antibody or a single domain antibody, etc. Suitable cell-surface molecule binding-molecules are proteinaceous molecules such as antibodies, sdAb, etc., and non-proteinaceous molecules, suitable for targeting a selected cell (type) and therewith suitable for bringing a saponin derivative of the invention and/or a payload, effector molecule, effector moiety such as a drug, toxin, oligonucleotide, enzyme, in close proximity of the target cell surface to which the cell-surface molecule binding-molecule can bind. Typically, the cell-surface molecule is a receptor. The binding of the cell-surface molecule binding-molecule to the target cell is followed by transfer of the saponin derivate and/or the payload, effector molecule, effector moiety, to the endosome of the cell to which the cell-surface molecule binding-molecule is bound.

Preferred is the first pharmaceutical combination comprising the second pharmaceutical composition or the third pharmaceutical composition according to the invention, wherein the second pharmaceutical composition or the third pharmaceutical composition comprise an antibody-toxin conjugate, an antibody-drug conjugate, or an antibody-oligonucleotide conjugate, wherein the antibody is a cell-surface molecule targeting molecule which can bind to a tumor-cell surface molecule, preferably to a tumor-cell receptor such as a tumor-cell specific receptor, more preferably to a receptor selected from CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, PSMA, CanAg, integrin-alphaV, CA6, CD33, mesothelin, Cripto, CD3, CD30, CD239, CD70, CD123, CD352, DLL3, CD25, ephrinA4, MUC1, Trop2, CEACAM5, CEACAM6, HER3, CD74, PTK7, Notch3, FGF2, C4.4A, FLT3, CD38, FGFR3, CD7, PD-L1, CTLA4, CD52, PDGFRA, VEGFR1, VEGFR2, preferably selected from CD71, HER2 and EGFR, more preferably wherein the antibody is a whole antibody or at least a cell-surface molecule binding fragment or -domain thereof, and preferably wherein the antibody comprises or consists of any one of cetuximab, daratumumab, gemtuzumab, trastuzumab, panitumumab, brentuximab, inotuzumab, moxetumomab, polatuzumab, obinutuzumab, OKT-9 anti-CD71 monoclonal antibody of the IgG type, pertuzumab, rituximab, ofatumumab, Herceptin, alemtuzumab, pinatuzumab, OKT-10 anti-CD38 monoclonal antibody, an antibody of Table A2, preferably cetuximab or trastuzumab or OKT-9, or at least one cell-surface molecule binding fragment or -domain thereof. Such an antibody can also be a binding derivative or binding fragment or binding domain thereof such as a F(ab′)2 fragment, Fab′ fragment, Fab fragment, scFv, dsFv, scFv-Fc, reduced IgG (rIgG), minibody, diabody, triabody, tetrabody, Fc fusion protein, nanobody, variable V domain, a single-domain antibody (sdAb), preferably a V_{HH}, a string of single-domain antibodies, a binding molecule comprising V_{HH}, a binding molecule comprising V_{H}, etc.

Preferred is the first pharmaceutical combination comprising the second pharmaceutical composition or the third pharmaceutical composition according to the invention wherein the effector moiety is an oligonucleotide comprising or consisting of any one or more of a nucleic acid and a xeno nucleic acid, preferably selected from any one or more of a vector, a gene, a cell suicide inducing transgene, deoxyribonucleic acid (DNA), ribonucleic acid (RNA), anti-sense oligonucleotide (ASO, AON), short interfering RNA (siRNA), microRNA (miRNA), DNA aptamer, RNA aptamer, mRNA, mini-circle DNA, peptide nucleic acid (PNA), phosphoramidate morpholino oligomer (PMO), locked nucleic acid (LNA), bridged nucleic acid (BNA), 2′-deoxy-2′-fluoroarabino nucleic acid (FANA), 2′-O-methoxyethyl-RNA (MOE), 2′-0,4′-aminoethylene bridged nucleic acid, 3′-fluoro hexitol nucleic acid (FHNA), a plasmid, glycol nucleic acid (GNA) and threose nucleic acid (TNA), or a derivative thereof, more preferably a BNA or an siRNA, for example a BNA for silencing HSP27 protein expression.

Also preferred is the first pharmaceutical combination comprising the second pharmaceutical composition or the third pharmaceutical composition according to the invention wherein the effector moiety comprises or consists of at least one proteinaceous molecule, preferably selected from any one or more of a peptide, a protein, an enzyme such as urease and Cre-recombinase, a ribosome-inactivating protein, a proteinaceous toxin selected from any one or more of a viral toxin such as apoptin; a bacterial toxin such as Shiga toxin, Shiga-like toxin, Pseudomonas aeruginosa exotoxin (PE) or exotoxin A of PE, full-length or truncated diphtheria toxin (DT), cholera toxin; a fungal toxin such as alpha-sarcin; a plant toxin including ribosome-inactivating proteins and the A chain of type 2 ribosome-inactivating proteins such as dianthin e.g. dianthin-30 or dianthin-32, saporin e.g. saporin-S3 or saporin-S6, bouganin or de-immunized derivative debouganin of bouganin, shiga-like toxin A, pokeweed antiviral protein, ricin, ricin A chain, modeccin, modeccin A chain, abrin, abrin A chain, volkensin, volkensin A chain, viscumin, viscumin A chain; or an animal or human toxin such as frog RNase, or granzyme B or angiogenin from humans, or any fragment or derivative thereof; preferably the protein toxin is dianthin and/or saporin.

An embodiment is the first pharmaceutical combination comprising the second pharmaceutical composition of the invention or the third pharmaceutical composition of the invention, wherein the second pharmaceutical composition or the third pharmaceutical composition comprises any one or more of an antibody-effector moiety conjugate, a receptor-ligand—effector moiety conjugate, an antibody-drug conjugate, a receptor-ligand—drug conjugate, an antibody-oligonucleotide conjugate or a receptor-ligand—oligonucleotide conjugate, wherein the drug is for example a toxin such as saporin and dianthin, and wherein the oligonucleotide is for example an siRNA or a BNA, for example for gene silencing of apolipoprotein B or HSP27.

An embodiment is the first pharmaceutical combination of the invention comprising the second pharmaceutical composition or the third pharmaceutical composition of the invention, wherein the saponin derivative is a saponin derivative selected from the group consisting of derivatives of: SO1861, SA1657, GE1741, SA1641, QS-21, QS-21A, QS-21 A-api, QS-21 A-xyl, QS-21B, QS-21 B-api, QS-21 B-xyl, QS-7-xyl, QS-7-api, QS-17-api, QS-17-xyl, QS1861, QS1862, Quillajasaponin, Saponinum album, QS-18, Quil-A, Gyp1, gypsoside A, AG1, AG2, SO1542, SO1584, SO1658, SO1674, SO1832, SO1862, SO1904, stereoisomers thereof and combinations thereof, preferably the saponin derivative is selected from the group consisting of an SO1861 derivative, a GE1741 derivative, an SA1641 derivative, a QS21 derivative, and a combination thereof, more preferably the saponin derivative is an SO1861 derivative or a QS21 derivative, more preferably, the saponin derivative is an SO1861 derivative, even more preferably the saponin derivative is according to formula (VIII).

An embodiment is the third pharmaceutical composition according to the invention comprising a saponin derivative according to the invention, preferably a pharmaceutically acceptable diluent, and further comprising:
a pharmaceutically acceptable salt, preferably a pharmaceutically acceptable inorganic salt, such as an ammonium, calcium, copper, iron, magnesium, manganese, potassium, sodium, strontium or zinc salt, preferably NaCl; and/or
a pharmaceutically acceptable buffer system, such as a phosphate, a borate, a citrate, a carbonate, a histidine, a lactate, a tromethamine, a gluconate, an aspartate, a glutamate, a tartarate, a succinate, a malate, a fumarate, an acetate and/or a ketoglutarate containing buffer system.

An embodiment is the third pharmaceutical composition according to the invention comprising a saponin derivative according to the invention and a pharmaceutically acceptable diluent, preferably water, wherein the composition is liquid at a temperature of 25° C. and has a pH within the range of 2-11, preferably within the range of 4-9, more preferably within the range of 6-8.

An embodiment is the third pharmaceutical composition according to the invention comprising a saponin derivative according to the invention and a pharmaceutically acceptable diluent, preferably water, wherein the composition is liquid at a temperature of 25° C. and wherein the concentration of the saponin derivative is within the range of 10-12 to 1 mol/I, preferably within the range of 10-9 to 0.1 mol/I, more preferably within the range of 10⁻⁶ to 0.1 mol/I.

An aspect of the invention relates to the first pharmaceutical composition of the invention, the first pharmaceutical combination comprising the first pharmaceutical composition of the invention or the third pharmaceutical composition of the invention, for use as a medicament.

In preferred embodiments there is provided the first pharmaceutical composition of the invention wherein the saponin derivative comprises, preferably consists of the saponin derivative according to formula (VIII), or the first pharmaceutical combination comprising the first pharmaceutical composition of the invention wherein the saponin derivative comprises, preferably consists of the saponin derivative according to formula (VIII), or the third pharmaceutical composition of the invention wherein the saponin derivative comprises, preferably consists the saponin derivative according to formula (VIII), for use as a medicament.

In another aspect of the invention there is provided the saponin derivative as described herein, preferably the saponin derivative according to formula (VIII) for use as a medicament.

An aspect of the invention relates to the first pharmaceutical composition of the invention, the first pharmaceutical combination comprising the first pharmaceutical composition of the invention, or the third pharmaceutical composition of the invention, for use in the treatment or prophylaxis of a cancer or an auto-immune disease, such as rheumatoid arthritis. In preferred embodiments there is provided the first pharmaceutical composition of the invention wherein the saponin derivative comprises, preferably consists of the saponin derivative according to formula (VIII), the first pharmaceutical combination of the invention wherein the saponin derivative comprises, preferably consists of the saponin derivative according to formula (VIII), or the third pharmaceutical composition of the invention wherein the saponin derivative comprises, preferably consists of the saponin derivative according to formula (VIII), for use in the treatment or prophylaxis of a cancer or an auto-immune disease.

Method for delivering an effector molecule inside a cell, using a saponin derivative

An aspect of the invention relates to an in vitro or ex vivo method for transferring a molecule from outside a cell to inside said cell, preferably into the cytosol of said cell, comprising the steps of:
a) providing a cell;
b) providing the molecule for transferring from outside the cell into the cell provided in step a);
c) providing a saponin derivative according to the invention;
d) contacting the cell of step a) in vitro or ex vivo with the molecule of step b) and the saponin derivative of step c), therewith establishing the transfer of the molecule from outside the cell into said cell.

An embodiment is the method of the invention, wherein the cell is a human cell such as a T-cell, an NK-cell, a tumor cell, and/or wherein the molecule of step b) is any one of: an antibody-drug conjugate, a receptor-ligand—drug conjugate, an antibody-oligonucleotide conjugate or a receptor-ligand—oligonucleotide conjugate, wherein the drug is for example a toxin and wherein the oligonucleotide is for example an siRNA or a BNA, and/or wherein the saponin derivative is selected from the group consisting of derivatives of: SO1861, SA1657, GE1741, SA1641, QS-21, QS-21A, QS-21 A-api, QS-21 A-xyl, QS-21B, QS-21 B-api, QS-21 B-xyl, QS-7-xyl, QS-7-api, QS-17-api, QS-17-xyl, QS1861, QS1862, Quillajasaponin, Saponinum album, QS-18, Quil-A, Gyp1, gypsoside A, AG1, AG2, SO1542, SO1584, SO1658, SO1674, SO1832, SO1862, SO1904, stereoisomers thereof and combinations thereof, preferably the saponin derivative is selected from the group consisting of an SO1861 derivative, a GE1741 derivative, an SA1641 derivative, a QS21 derivative, and a combination thereof, more preferably the saponin derivative is an SO1861 derivative or an QS21 derivative, most preferably, the saponin derivative is an SO1861 derivative; or wherein the saponin derivative is according to formula (VIII).

In particular embodiments the in vitro or ex vivo method for transferring a molecule from outside a cell to inside said cell, preferably into the cytosol of said cell as described herein is provided wherein the saponin derivative comprises, preferably consists of the saponin derivative according to formula (VIII).

Saponin conjugate based on the saponin derivatives

An aspect of the invention relates to a saponin conjugate comprising a cell-surface molecule binding-molecule such as a first proteinaceous molecule (‘proteinaceous molecule 1’) or a second proteinaceous molecule (‘proteinaceous molecule 2’) that is covalently bound to the saponin derivative according to the invention, i.e. covalently linked to the saponin derivative. The cell-surface molecule binding-molecule is covalently bound to the derivatisation in the saponin derivative, i.e. the derivatised aldehyde functional group of the saponin on which the saponin derivative is based. In the saponin conjugate, the cell-surface molecule binding-molecule is bound to the saponin hydrazone functional group (=N—N(H)—C(O)—), either directly, or through a linker, such as LINKER A. The cell-surface molecule binding-molecule typically is a protein, such as an antibody or a binding fragment thereof, or a single-domain antibody (sdAb) or a binding molecule comprising a sdAb.

An aspect of the invention relates to a saponin conjugate based on a saponin derivative according to the invention, wherein R³ is a cyclooctyne moiety selected from the group consisting of cyclooctyne moieties (II)a-d,

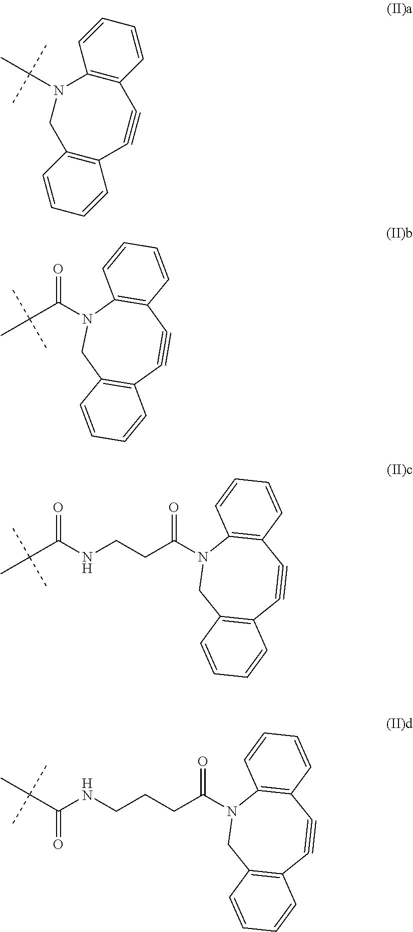

and wherein the cyclooctyne moiety is transformed to a triazole functional group through reaction with a first proteinaceous molecule (‘proteinaceous molecule 1’) comprising an azide functional group according to formula (IX)

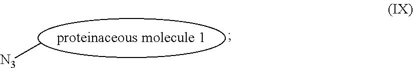

or

wherein R³ is a linker according to formula (III):

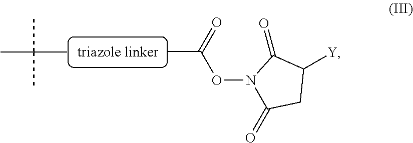

wherein Y is H or SO₂ONa, preferably H,
the linker according to formula (III) is selected from the group consisting of linkers (IV)a-j:

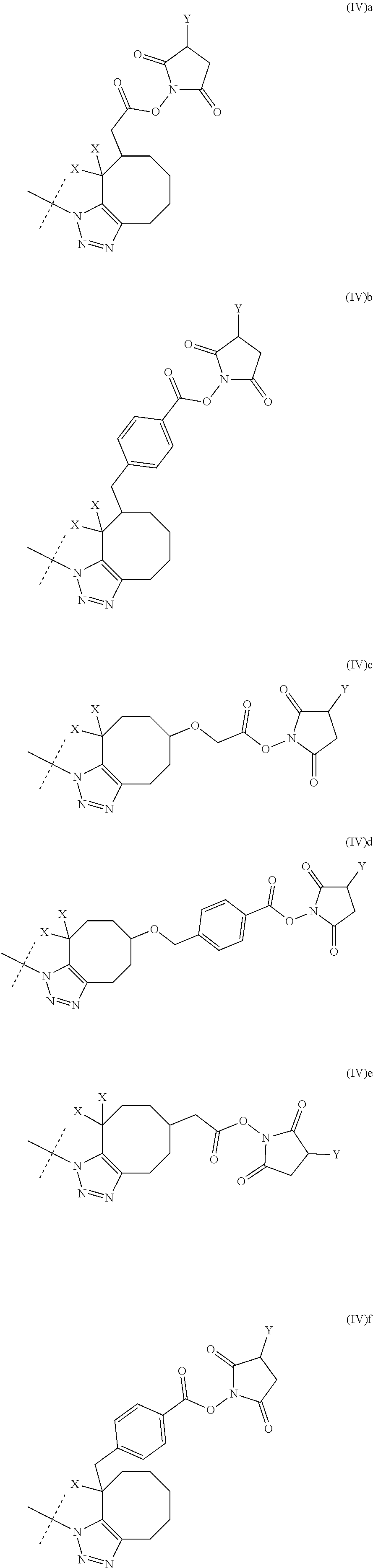

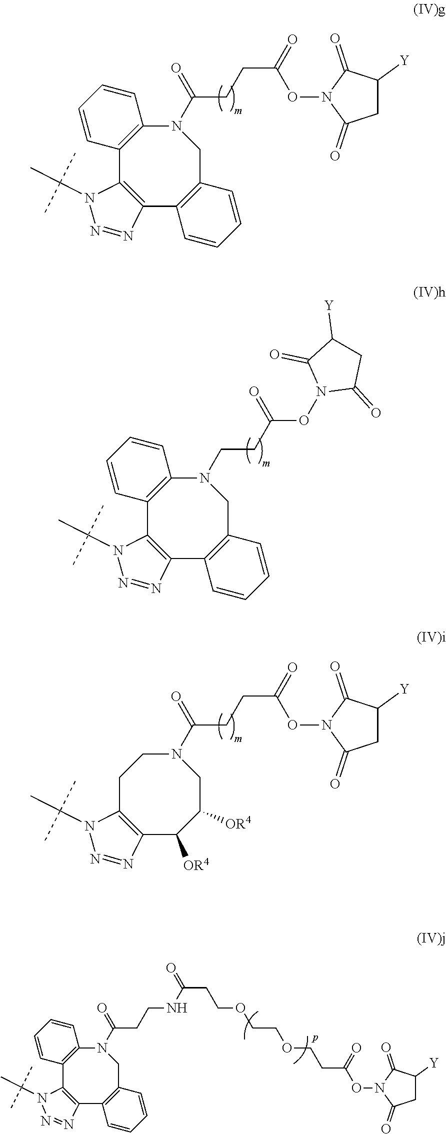

wherein X is H or F, preferably F;
m is 1 or 2;
p is an integer selected from 0-5, preferably 1→4, more preferably 2-3;
R⁴ is a C1-C3 alkyl chain, preferably a C1 alkyl chain; and
wherein the N-hydroxy succinimide active ester functional group

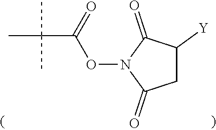

is transformed to an amide functional group (—C(O)—N(H)—) through reaction with a second proteinaceous molecule (‘proteinaceous molecule 2’) comprising an amine functional group according to formula (XI)

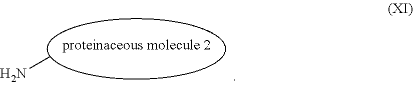

An embodiment is the saponin conjugate of the invention based on a saponin derivative according to the invention wherein the aldehyde functional group is transformed to a hydrazone functional group according to according to formula (A):

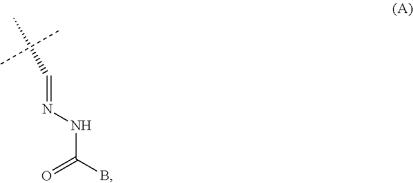

wherein B is the LINKER A wherein the LINKER A is according to the structure (AA4):
LINKER A-R³
(AA4),
and R³ is as here above defined for the saponin conjugate of the invention.

The saponin derivatives according to formula (V), wherein R³ is a cyclooctyne moiety selected from the group consisting of cyclooctyne moieties (II)a-d

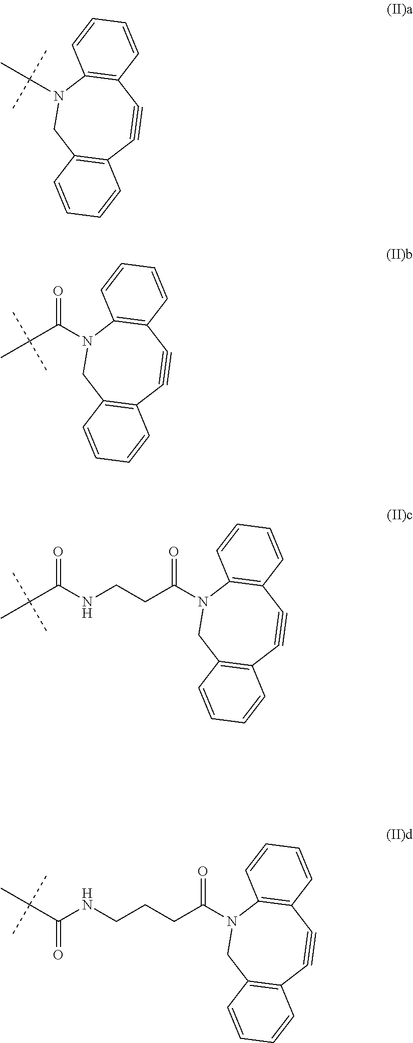

are suitable for application as a precursor for a conjugation reaction with a further molecule comprising a free azide group. The cyclooctyne moieties (II)a-d of the saponin derivative can form a triazole functional group with such a free azide group. For example, the saponin derivatives according to formula (V), wherein R³ is a cyclooctyne moiety selected from the group consisting of cyclooctyne moieties (II)a-d, can be covalently coupled to a peptide or a protein which comprises a free azide group. Such a protein is for example an antibody or a binding derivative or binding fragment or binding domain thereof such as a F(ab′)2 fragment, Fab′ fragment, Fab fragment, scFv, dsFv, scFv-Fc, reduced IgG (rIgG), minibody, diabody, triabody, tetrabody, Fc fusion protein, nanobody, variable V domain, a single-domain antibody (sdAb), preferably a V_{HH}, for example camelid V_{H}, or a ligand for a cell-surface molecule such as a receptor such as EGF and a cytokine.. Application of the saponin derivative according to formula (V), wherein R³ is a cyclooctyne moiety selected from the group consisting of cyclooctyne moieties (II)a-d, in a coupling reaction with e.g. an antibody that comprises a free azide group, provides a conjugate for targeted delivery of the saponin to and inside a cell, when the antibody (or the binding domain or fragment thereof) is an antibody for specific binding to a target cell surface molecule such as a receptor, e.g. as present on a tumor cell. Preferably, the saponin derivative is coupled to an antibody or at least one V_{HH} capable of binding to a tumor-cell specific surface molecule such as a receptor, e.g. HER2, EGFR, CD71.

The saponin derivatives according to formula (VII)

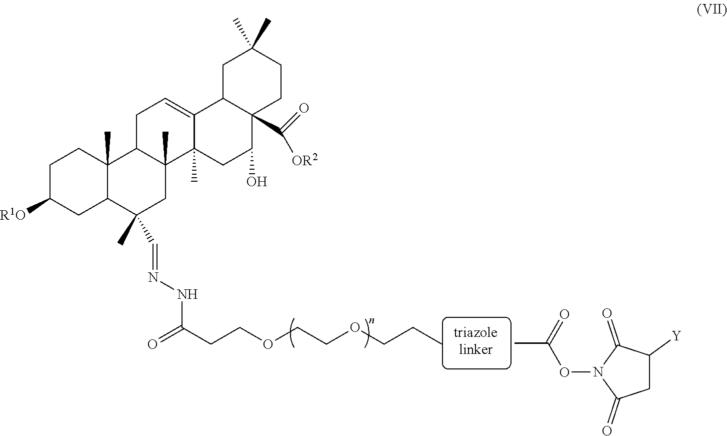

are suitable for application as a precursor for a conjugation reaction with a further molecule comprising a free amino group. The N-hydroxy succinimide active ester functional group

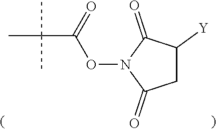

is transformed to of the saponin derivative according to formula (VII) can form an amide bond (—C(O)—N(H)—) with such a free amino group. For example, the saponin derivative according to formula (VII) can be covalently coupled to a peptide or a protein which comprises a free amino group such as a lysine with a free amino group. Such a protein is for example an antibody or a binding derivative or binding fragment or binding domain thereof such as a F(ab′)2 fragment, Fab′ fragment, Fab fragment, scFv, dsFv, scFv-Fc, reduced IgG (rIgG), minibody, diabody, triabody, tetrabody, Fc fusion protein, nanobody, variable V domain, a single-domain antibody (sdAb), preferably a V_{HH}, for example camelid V_{H}, or a ligand for a cell-surface molecule such as a receptor such as EGF and a cytokine. Application of the saponin derivative according to formula (VII) in a coupling reaction with e.g. an antibody that comprises a free amino group, provides a conjugate for targeted delivery of the saponin to and inside a cell, when the antibody (or the binding domain or fragment thereof) is an antibody for specific binding to a target cell surface molecule such as a receptor, e.g. as present on a tumor cell. Preferably, the saponin derivative is coupled to an antibody or V_{HH} capable of binding to a tumor-cell specific surface molecule such as a receptor, e.g. HER2, EGFR, CD71.

It will appreciated by the skilled person that since the saponin conjugate of the invention comprises (is based on) the saponin derivative according to the invention, all embodiments referring to the saponin derivative applies mutatis mutandis to the saponin conjugate. That is to say, the saponin conjugates of the invention are based on the saponin derivatives according to the invention, detailed here above.

A preferred embodiment is the saponin conjugate according to the invention, wherein the saponin conjugate is according to formula (X),

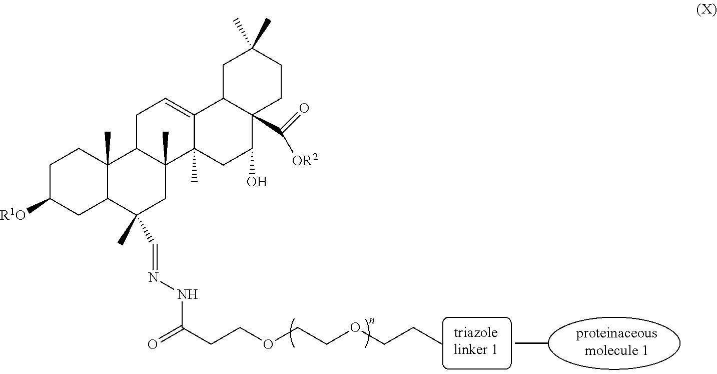

wherein R¹ and R² are independently selected from hydrogen, a monosaccharide, a linear oligosaccharide and a branched oligosaccharide,
n is an integer selected from 0-15,
the triazole linker 1 is selected from the group of triazole linkers (XI)a-d,

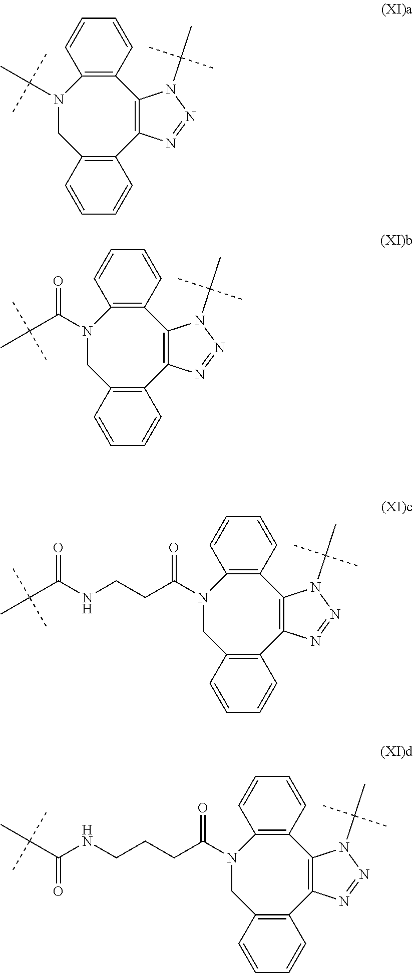

or
wherein the saponin conjugate is according to formula (XII),

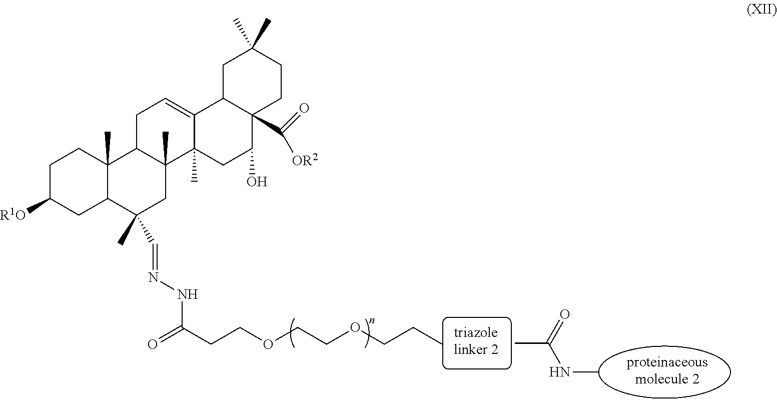

wherein R¹ and R² are independently selected from hydrogen, a monosaccharide, a linear oligosaccharide and a branched oligosaccharide,
n is an integer selected from 0-15,
the triazole linker 2 is selected from the group consisting of triazole linkers (XIII)a-j

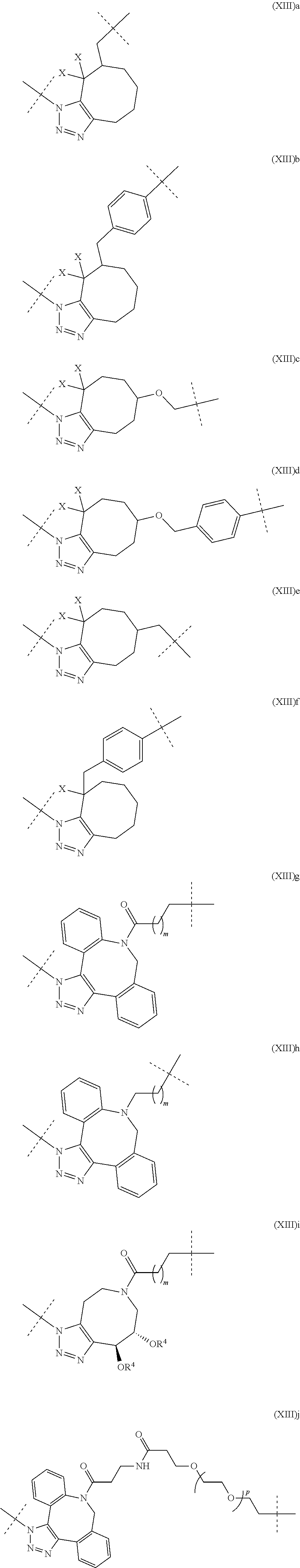

wherein X is H or F, preferably F;
m is 1 or 2, preferably 1;
p is an integer selected from 0-5, preferably 1→4, more preferably 2-3; and
R⁴ is a C1-C3 alkyl chain, preferably a C1 alkyl chain.

Preferred is the saponin conjugate according to the invention, wherein the conjugate is according to formula (XII)

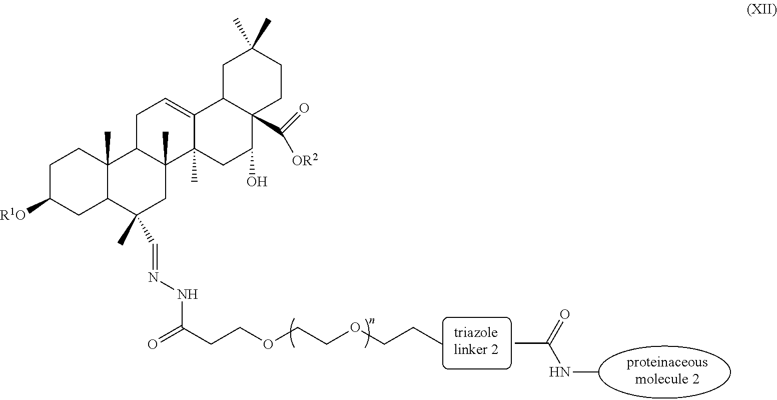

wherein R¹ and R² are independently selected from hydrogen, a monosaccharide, a linear oligosaccharide and a branched oligosaccharide, selected from the list of saccharides R¹ and saccharides R² that are present in the saponin derivative of the invention (see here above),
n is an integer selected from 0-14,
the triazole linker 2 is selected from the group consisting of triazole linkers (XIII)a-j, preferably selected from the group consisting of linkers (XIII)g-(XIII)j, more preferably the linker is linker (XIII)g or (XIII)h, even more preferably the linker is linker (XIII)g;

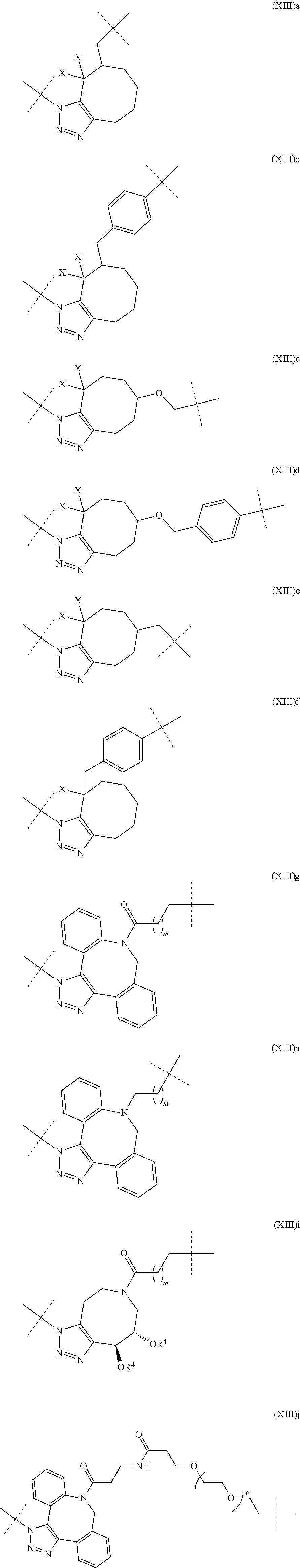

wherein X is H or F, preferably F;
m is 1 or 2, preferably 1;
p is an integer selected from 0-5, preferably 1→4, more preferably 2-3; and
R⁴ is a C1-C3 alkyl chain, preferably a C1 alkyl chain.

Preferred is the saponin conjugate according to the invention, wherein the saponin conjugate is according to formula (X) or the saponin conjugate according to formula (XII), wherein n is an integer selected from 1-14, preferably 2-12, more preferably 3-9. A preferred embodiment is the saponin conjugate according to the invention, wherein the saponin conjugate is according to formula (X) or the saponin conjugate according to formula (XII), wherein n is 3.

A highly preferred embodiment is the saponin conjugate according to the invention, wherein the saponin conjugate is a compound according to formula (XIV)

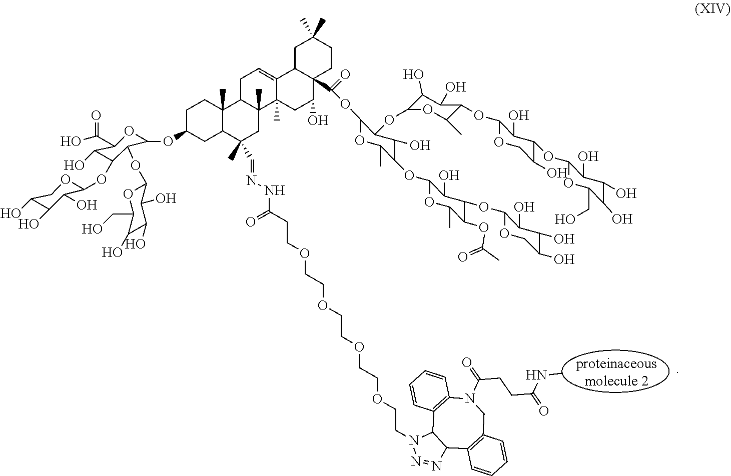

An embodiment is the saponin conjugate according to the invention, wherein the proteinaceous molecule 1 and the proteinaceous molecule 2 comprise a same first binding site for binding to a first epitope of a first cell-surface molecule, or wherein the proteinaceous molecule 1 comprises the first binding site for binding to a first epitope of a first cell-surface molecule and the proteinaceous molecule 2 comprises a second binding site for binding to a second epitope of a second cell-surface molecule, wherein the first binding site and the second binding site are different, and optionally wherein the first cell surface molecule and the second cell surface molecule are present on the same cell, preferably the first cell surface molecule and the second cell surface molecule are present on the same cell.

An embodiment is the saponin conjugate according to the invention, wherein the first binding site of the proteinaceous molecule 1 and the proteinaceous molecule 2 and, if present, the second binding site of the proteinaceous molecule 2 is selected from any one or more of cell-surface molecule binding-molecules: an amino acid, a peptide, a protein, an antibody or a derivative or fragment thereof such as a Fab or an scFv, a single-domain antibody (sdAb), a ligand such as EGF, an adnectin, an affibody, an anticalin, or binding molecules comprising one or more of any of these cell-surface molecule binding-molecules.

An embodiment is the saponin conjugate according to the invention, wherein the first binding site of the proteinaceous molecule 1 and the proteinaceous molecule 2 and, if present, the second binding site of the proteinaceous molecule 2 is/are or comprise(s) a single-domain antibody (sdAb), preferably V_{H} domain derived from a heavy chain of an antibody, preferably of immunoglobulin G origin, preferably of human origin, a V_{L} domain derived from a light chain of an antibody, preferably of immunoglobulin G origin, preferably of human origin, a V_{HH} domain such as derived from a heavy-chain only antibody (HCAb) such as from Camelidae origin or Ig-NAR origin such as a variable heavy chain new antigen receptor (VNAR) domain, preferably the HCAb is from Camelidae origin, preferably the sdAb is a V_{HH} domain derived from an HCAb from Camelidae origin (camelid V_{H}) such as derived from an HCAb from camel, lama, alpaca, dromedary, vicuna, guanaco and Bactrian camel.

An embodiment is the saponin conjugate according to the invention, wherein the first epitope of the first cell-surface molecule to which the first binding site can bind is a tumor-cell specific first epitope of a first tumor-cell surface molecule, more preferably a tumor-cell specific first epitope of a first tumor-cell surface receptor specifically present on a tumor cell, and wherein the second epitope of the second cell-surface molecule is a tumor-cell specific second epitope of a second tumor-cell surface molecule, more preferably a tumor-cell specific second epitope of a second tumor-cell surface receptor specifically present on a tumor cell, wherein the first cell-surface molecule and the second cell-surface molecule are present on the same cell.

An embodiment is the saponin conjugate according to the invention, wherein the cell-surface molecule targeting (binding) molecule can bind to a tumor-cell surface molecule, preferably a tumor-cell receptor such as a tumor-cell specific receptor, more preferably a receptor selected from CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, PSMA, CanAg, integrin-alphaV, CA6, CD33, mesothelin, Cripto, CD3, CD30, CD239, CD70, CD123, CD352, DLL3, CD25, ephrinA4, MUC1, Trop2, CEACAM5, CEACAM6, HER3, CD74, PTK7, Notch3, FGF2, C4.4A, FLT3, CD38, FGFR3, CD7, PD-L1, CTLA4, CD52, PDGFRA, VEGFR1, VEGFR2, preferably selected from CD71, HER2 and EGFR, more preferably wherein the cell-surface molecule targeting (binding) molecule is or comprises a monoclonal antibody or at least one cell-surface molecule binding fragment or -domain thereof, and preferably comprises or consists of any one of cetuximab, daratumumab, gemtuzumab, trastuzumab, panitumumab, brentuximab, inotuzumab, moxetumomab, polatuzumab, obinutuzumab, OKT-9 anti-CD71 monoclonal antibody of the IgG type, pertuzumab, rituximab, ofatumumab, Herceptin, alemtuzumab, pinatuzumab, OKT-10 anti-CD38 monoclonal antibody, an antibody of Table A2, preferably cetuximab or trastuzumab or OKT-9, or at least one cell-surface molecule binding fragment or -domain thereof.

An embodiment is the saponin conjugate or the saponin derivative according to the invention, wherein the hydrazone functional group (═N—N(H)—C(O)—) is cleavable or hydrolysable under acidic conditions, preferably at pH 4.0-6.5, such that the aldehyde group of the saponin on which the saponin derivative is based is formed upon cleavage of the hydrazone functional group. Since the hydrazone functional group is not hydrolysed at physiological pH as apparent in e.g. the circulation and in tissue of mammals, e.g. human subjects, the saponin in the saponin conjugate is not split off from the conjugate while in the circulation or in tissue. Once the cell-surface molecule binding-molecule of the saponin conjugate, e.g. an antibody, bound to the cell-surface molecule on the target cell, the saponin conjugate is endocytosed by the cell and the saponin conjugate is transferred to and delivered into the cell endosome. In the endosome, the pH is suitable for hydrolysis (cleavage) of the hydrazone functional group, such that the aldehyde functional group of the saponin on which the saponin conjugate is based, is again formed and the free saponin is provided. The free saponin in the endosome facilitates endosomal escape of any effector molecule or effector moiety into the cytosol, when such effector molecule or effector moiety is co-localized in the endosome. Without wishing to be bound by any theory, conjugating the saponin to a binding molecule through the hydrazone functional group efficiently and sufficiently masks the cytotoxic activity of the free saponin on which the conjugate is based, by altering the initial aldehyde functional group of the saponin into the hydrazone functional group. Once in the endosome, the hydrazone functional group is susceptible to hydrolysis at the endosomal acidic pH, stimulating formation of the aldehyde functional group in the saponin, therewith providing the ‘active’ form of the saponin, when endosomal escape enhancing activity is concerned. Once inside the endosome of e.g. an auto-immune cell or a tumor cell, any cytotoxicity of the free saponin comprising the aldehyde functional group is not anymore hampering the benefits of the improved delivery of any effector moiety or molecule in the cytosol. That is to say, in the saponin conjugate, cytotoxicity of the saponin is efficiently blocked, inhibited or diminished, when the conjugate circulates or is present in the body extracellularly, and once inside the endosome, the formed free saponin comprising the aldehyde functional group is an efficient molecule for potentiating a desired effect of an effector molecule or moiety (that is for example co-administered to a subject in the form of for example an ADC, AOC, and capable of binding to the same cell as to which the saponin-conjugate of the invention can bind).

An embodiment is the saponin conjugate or the saponin derivative according to the invention, wherein the hydrazone functional group is cleavable or hydrolysable in vivo under acidic conditions as present in endosomes and/or lysosomes of mammalian cells, preferably human cells, more preferably human aberrant cells such as diseased cells, tumor cells, auto-immune cells, preferably at pH 4.0-6.5, and more preferably at pH 5.5, such that the aldehyde group of the saponin on which the saponin derivative is based is formed upon hydrolysis of the hydrazone functional group.

Pharmaceutical combination and pharmaceutical composition comprising a saponin conjugate

An aspect of the invention relates to a second pharmaceutical combination comprising:
a) a fourth pharmaceutical composition comprising the saponin conjugate according to the invention and optionally a pharmaceutically acceptable excipient and/or diluent, wherein the proteinaceous molecule 1 and the proteinaceous molecule 2 comprise a same first binding site for binding to a first epitope of a first cell-surface molecule, or wherein the proteinaceous molecule 1 comprises the first binding site for binding to a first epitope of a first cell-surface molecule and the proteinaceous molecule 2 comprises a second binding site for binding to a second epitope of a second cell-surface molecule, wherein the first binding site and the second binding site are different, and optionally wherein the first cell surface molecule and the second cell surface molecule are present on the same cell, preferably the first cell surface molecule and the second cell surface molecule are present on the same cell; and
b) a fifth pharmaceutical composition comprising a conjugate comprising a cell-surface molecule binding-molecule, such as a third proteinaceous molecule (′proteinaceous molecule 3′), and an effector moiety, wherein the proteinaceous molecule 3 is the same or different from the proteinaceous molecule 1 and the proteinaceous molecule 2 present in the saponin conjugate, the proteinaceous molecule 3 comprising a third binding site for binding to a third epitope of a third cell-surface molecule, wherein the third cell-surface molecule, if different from the first cell surface molecule and/or the second cell surface molecule, is present on the same cell as the first cell surface molecule and the second cell surface molecule, the fifth pharmaceutical composition optionally further comprising a pharmaceutically acceptable excipient and/or diluent.

An embodiment is the fourth pharmaceutical composition according to the invention comprising the saponin conjugate according to the invention, preferably a pharmaceutically acceptable diluent, and further comprising:
a pharmaceutically acceptable salt, preferably a pharmaceutically acceptable inorganic salt, such as an ammonium, calcium, copper, iron, magnesium, manganese, potassium, sodium, strontium or zinc salt, preferably NaCl; and/or a pharmaceutically acceptable buffer system, such as a phosphate, a borate, a citrate,
a carbonate, a histidine, a lactate, a tromethamine, a gluconate, an aspartate, a glutamate, a tartarate, a succinate, a malate, a fumarate, an acetate and/or a ketoglutarate containing buffer system.

An embodiment is the fourth pharmaceutical composition according to the invention comprising the saponin conjugate according to the invention and a pharmaceutically acceptable diluent, preferably water, wherein the composition is liquid at a temperature of 25° C. and has a pH within the range of 2-11, preferably within the range of 4-9, more preferably within the range of 6-8.

An embodiment is the first pharmaceutical composition of the invention, wherein the saponin conjugate is the saponin conjugate according to formula (XIV).

An embodiment is the second pharmaceutical combination according to the invention, comprising:
(a) the fourth pharmaceutical composition according to the invention comprising the saponin conjugate according to the invention, wherein the first epitope on the first cell-surface molecule is a tumor-cell specific first epitope on a first tumor cell-specific surface molecule, preferably a tumor-cell specific first epitope on a first cell-surface receptor specifically present at a tumor cell; and
(b) the fifth pharmaceutical composition according to the invention, wherein the third cell-surface molecule is a third tumor cell-specific surface molecule which is the same or different from the first tumor cell-specific surface molecule, preferably a third cell-surface receptor specifically present at a tumor cell which is the same or different from the first cell-surface receptor specifically present at said tumor cell, and wherein the third epitope is a tumor-cell specific third epitope, wherein the first epitope and the third epitope, which is the same as or different from the first epitope, are exposed at the same cell.

An aspect of the invention relates to a third pharmaceutical combination comprising:
a) a fourth pharmaceutical composition comprising the saponin conjugate according to the invention and optionally a pharmaceutically acceptable excipient and/or diluent, wherein the proteinaceous molecule 1 and the proteinaceous molecule 2 comprise a same first binding site for binding to a first epitope of a first cell-surface molecule, or wherein the proteinaceous molecule 1 comprises the first binding site for binding to a first epitope of a first cell-surface molecule and the proteinaceous molecule 2 comprises a second binding site for binding to a second epitope of a second cell-surface molecule, wherein the first binding site and the second binding site are different, and optionally wherein the first cell surface molecule and the second cell surface molecule are present on the same cell, preferably the first cell surface molecule and the second cell surface molecule are present on the same cell; and
b) a sixth pharmaceutical composition comprising a conjugate comprising a cell-surface molecule binding-molecule, such as a fourth proteinaceous molecule (‘proteinaceous molecule 4’), and an effector moiety, wherein the proteinaceous molecule 4 comprises the first binding site for binding to the first epitope on the cell-surface molecule of (a), the sixth pharmaceutical composition optionally further comprising a pharmaceutically acceptable excipient and/or diluent, wherein the first binding site of the proteinaceous molecule 1 or proteinaceous molecule 2 and the first binding site of the proteinaceous molecule 4 are the same, and wherein the first cell-surface molecule and the first epitope on the first cell-surface molecule, to which the proteinaceous molecule 1 or the proteinaceous molecule 2 can bind, and the first cell-surface molecule and the first epitope on the first cell-surface molecule, to which the proteinaceous molecule 4 can bind, are the same.

An embodiment is the second pharmaceutical combination or the third pharmaceutical combination according to the invention, wherein the proteinaceous molecule 3 and the proteinaceous molecule 4 is of a type as detailed or the proteinaceous molecule 1 and proteinaceous molecule 2, e.g. an antibody or an antigen binding fragment or antigen binding domain thereof, a ligand for a cell-surface molecule or cell-surface receptor such as EGF or a cytokine, an scFv, a Fab, a binding molecule comprising an sdAb, such as a V_{HH}, a binding molecule comprising any of an adnectin, an anticalin, an affibody. The skilled person will appreciate that for the proteinaceous molecule 1 and proteinaceous molecule 2 and proteinaceous molecule 3 and proteinaceous molecule 4 any cell-surface molecule binding-molecule can be selected and is suitable for application in the saponin conjugates of the invention and for application in the pharmaceutical compositions and combinations of the invention, that is known today in the technological field of (specifically) targeting a mammalian (aberrant, tumor, auto-immune, etc.) cell with a binding molecule, e.g. for (targeted and specific) delivery of an effector molecule or effector moiety conjugated to the cell-surface molecule binding-molecule. Typical examples of such suitable cell-surface molecule binding-molecule are the antibodies and binding fragments, binding domains and binding derivatives thereof and ligands such as EGF or cytokines used today to deliver drug molecules, payloads, effector moieties, protein toxins, small-molecule toxins, enzymes, oligonucleotides, etc., etc., e.g. in ADCs and AOCs. Any cell-surface molecule binding-molecule applicable for e.g. designing and providing ADCs and AOCs is typically also applicable for providing the saponin conjugate of the invention (e.g. proteinaceous molecule 1 and proteinaceous molecule 2). Any cell-surface molecule binding-molecule applicable for e.g. designing and providing ADCs and AOCs is typically also applicable for providing the cell-surface molecule binding-molecule conjugated with an effector moiety, e.g. the proteinaceous molecule 3 and proteinaceous molecule 4 conjugated to an effector moiety. The skilled person will also appreciate that it is within the scope of the invention that for the proteinaceous molecule 1 and proteinaceous molecule 2 and proteinaceous molecule 3 and proteinaceous molecule 4 any cell-surface molecule binding-molecule can be selected and is suitable for application in the saponin conjugates of the invention and for application in the pharmaceutical compositions and combinations of the invention, that is known today in the technological field of (specifically) targeting a mammalian (aberrant, tumor, auto-immune, etc.) cell with a binding molecule, e.g. for (targeted and specific) delivery of an effector molecule or effector moiety conjugated to the cell-surface molecule binding-molecule, wherein the cell-surface molecule binding-molecule is either a proteinaceous molecule or a non-proteinaceous molecule. That is to say, according to the invention, any of the cell-surface molecule binding-molecules proteinaceous molecule 1, proteinaceous molecule 2, proteinaceous molecule 3 and proteinaceous molecule 4 can be replaced by a non-proteinaceous cell-surface molecule binding-molecule suitable for targeting (binding to) a selected and desired cell such as a mammalian cell, e.g. a tumor cell or an auto-immune cell. According to the invention, it is preferred that the cell-surface molecules to which combinations of the proteinaceous molecule 1, proteinaceous molecule 2, proteinaceous molecule 3 and proteinaceous molecule 4 bind, are present at the surface of the same target cell, when combinations of proteinaceous molecule 1, proteinaceous molecule 2, proteinaceous molecule 3 and proteinaceous molecule 4 bind to different cell-surface molecules. For example, according to the invention, if in pharmaceutical combinations and compositions proteinaceous molecule 1 binds to Her2 and proteinaceous molecule 3 binds to CD71 or EGFR, Her2 and CD71 or Her2 and EGFR are typically present at the same cell.

An embodiment is the fourth pharmaceutical composition according to the invention, further comprising:
a pharmaceutically acceptable salt, preferably a pharmaceutically acceptable inorganic salt, such as an ammonium, calcium, copper, iron, magnesium, manganese, potassium, sodium, strontium or zinc salt, preferably NaCl; and/or
a pharmaceutically acceptable buffer system, such as a phosphate, a borate, a citrate, a carbonate, a histidine, a lactate, a tromethamine, a gluconate, an aspartate, a glutamate, a tartarate, a succinate, a malate, a fumarate, an acetate and/or a ketoglutarate containing buffer system.

An embodiment is the fourth pharmaceutical composition according to the invention comprising the saponin conjugate according to the invention and a pharmaceutically acceptable diluent, preferably water, wherein the composition is liquid at a temperature of 25° C. and has a pH within the range of 2-11, preferably within the range of 4-9, more preferably within the range of 6-8.

An aspect of the invention relates to a fourth pharmaceutical combination comprising:
a) the fourth pharmaceutical composition according to the invention; and
b) the sixth pharmaceutical composition according to the invention.

Preferred is the fourth pharmaceutical combination, wherein the first cell-surface molecule is expressed on a tumor cell surface, and preferably the first cell-surface molecule is a tumor cell-specific surface molecule, and wherein preferably the first epitope is a first tumor-cell specific epitope.

An embodiment is the second pharmaceutical combination or third pharmaceutical combination or fourth pharmaceutical combination according to the invention, wherein the third binding site of the proteinaceous molecule 3 and/or the first binding site of the proteinaceous molecule 4 comprises or consists of an antibody or a binding derivative or binding fragment or binding domain thereof such as a F(ab′)2 fragment, Fab′ fragment, Fab fragment, scFv, dsFv, scFv-Fc, reduced IgG (rIgG), minibody, diabody, triabody, tetrabody, Fc fusion protein, nanobody, variable V domain, a single-domain antibody (sdAb), preferably a V_{HH}, for example camelid V_{H}, or a ligand for a cell-surface molecule such as a receptor such as EGF and a cytokine, preferably a V_{H} domain derived from a heavy chain of an antibody, preferably of immunoglobulin G origin, preferably of human origin, a V_{L} domain derived from a light chain of an antibody, preferably of immunoglobulin G origin, preferably of human origin, a V_{HH} domain such as derived from a heavy-chain only antibody (HCAb) such as from Camelidae origin or Ig-NAR origin such as a variable heavy chain new antigen receptor (VNAR) domain, preferably the HCAb is from Camelidae origin, preferably the sdAb is a V_{HH} domain derived from an HCAb from Camelidae origin (camelid V_{H}) such as derived from an HCAb from camel, lama, alpaca, dromedary, vicuna, guanaco and Bactrian camel.

An aspect of the invention relates to a seventh pharmaceutical composition comprising the saponin conjugate according to the invention and the conjugate comprising proteinaceous molecule 3 and an effector moiety, or the conjugate comprising proteinaceous molecule 4 and an effector moiety, and optionally further comprising a pharmaceutically acceptable excipient and/or diluent.

Preferably, in the pharmaceutical combinations and in the pharmaceutical compositions, the effector moiety, when present, comprises or consists of any one or more of an oligonucleotide, a nucleic acid and a xeno nucleic acid, preferably selected from any one or more of a vector, a gene, a cell suicide inducing transgene, deoxyribonucleic acid (DNA), ribonucleic acid (RNA), anti-sense oligonucleotide (ASO, AON), short interfering RNA (siRNA), microRNA (miRNA), DNA aptamer, RNA aptamer, mRNA, mini-circle DNA, peptide nucleic acid (PNA), phosphoramidate morpholino oligomer (PMO), locked nucleic acid (LNA), bridged nucleic acid (BNA), 2′-deoxy-2′-fluoroarabino nucleic acid (FANA), 2′-O-methoxyethyl-RNA (MOE), 2′-0,4′-aminoethylene bridged nucleic acid, 3′-fluoro hexitol nucleic acid (FHNA), a plasmid, glycol nucleic acid (GNA) and threose nucleic acid (TNA), or a derivative thereof, more preferably a BNA, for example a BNA for silencing HSP27 protein expression.

Also preferably, in the pharmaceutical combinations and in the pharmaceutical compositions, the effector moiety, when present, comprises or consists of at least one proteinaceous molecule, preferably selected from any one or more of a peptide, a protein, an enzyme such as urease and Cre-recombinase, a protein toxin, a ribosome-inactivating protein, a proteinaceous toxin selected from any one or more of a viral toxin such as apoptin; a bacterial toxin such as Shiga toxin, Shiga-like toxin, Pseudomonas aeruginosa exotoxin (PE) or exotoxin A of PE, full-length or truncated diphtheria toxin (DT), cholera toxin; a fungal toxin such as alpha-sarcin; a plant toxin including ribosome-inactivating proteins and the A chain of type 2 ribosome-inactivating proteins such as dianthin e.g. dianthin-30 or dianthin-32, saporin e.g. saporin-S3 or saporin-S6, bouganin or de-immunized derivative debouganin of bouganin, shiga-like toxin A, pokeweed antiviral protein, ricin, ricin A chain, modeccin, modeccin A chain, abrin, abrin A chain, volkensin, volkensin A chain, viscumin, viscumin A chain; or an animal or human toxin such as frog RNase, or granzyme B or angiogenin from humans, or any fragment or derivative thereof; preferably the protein toxin is dianthin and/or saporin.

Also preferably, in the pharmaceutical combinations and in the pharmaceutical compositions, the effector moiety, when present, comprises or consists of at least one payload, preferably selected from any one or more of a toxin targeting ribosomes, a toxin targeting elongation factors, a toxin targeting tubulin, a toxin targeting DNA and a toxin targeting RNA, more preferably any one or more of emtansine, pasudotox, maytansinoid derivative DM1, maytansinoid derivative DM4, monomethyl auristatin E (MMAE, vedotin), monomethyl auristatin F (MMAF, mafodotin), a Calicheamicin, N-Acetyl-y-calicheamicin, a pyrrolobenzodiazepine (PBD) dimer, a benzodiazepine, a CC-1065 analogue, a duocarmycin, Doxorubicin, paclitaxel, docetaxel, cisplatin, cyclophosphamide, etoposide, docetaxel, 5-fluorouracyl (5-FU), mitoxantrone, a tubulysin, an indolinobenzodiazepine, AZ13599185, a cryptophycin, rhizoxin, methotrexate, an anthracycline, a camptothecin analogue, SN-38, DX-8951f, exatecan mesylate, truncated form of Pseudomonas aeruginosa exotoxin (PE38), a Duocarmycin derivative, an amanitin, a-amanitin, a spliceostatin, a thailanstatin, ozogamicin, tesirine, Amberstatin269 and soravtansine, or a derivative thereof.

An aspect of the invention relates to the second pharmaceutical combination or third pharmaceutical combination or fourth pharmaceutical combination according to the invention or the seventh pharmaceutical composition according to the invention, for use as a medicament.

An aspect of the invention relates to the second pharmaceutical combination or third pharmaceutical combination or fourth pharmaceutical combination according to the invention or the seventh pharmaceutical composition according to the invention, for use in the treatment or prevention of a cancer or of an autoimmune disease, such as rheumatoid arthritis.

TABLE A1
Saponins displaying (late) endosomal/lysosomal escape enhancing activity, and saponins comprising a structure
reminiscent to such saponins displaying (late) endosomal/lysosomal escape enhancing activity

| | | | Carbohydrate substituent at the |
|---|---|---|---|
| Saponin Name | Aglycone core | | C-28-OH group |
| | | Carbohydrate substituent | |
| | | at the C-3beta-OH group | |
| NP-005236 | 2alpha- | GlcA- | Glc/Gal- |
| | Hydroxyoleanolic | | |
| | acid | | |
| AMA-1 | 16alpha- | Glc- | Rha-(1→2)-[Xyl-(1→4)]-Rha- |
| | Hydroxyoleanolic | | |
| | acid | | |
| AMR | 16alpha- | Glc- | Rha-(1→2)-[Ara-(1→3)-Xyl-(1→4)]-Rha- |
| | Hydroxyoleanolic | | |
| | acid | | |
| alpha-Hederin | Hederagenin (23- | Rha-(1→2)-Ara- | — |
| | Hydroxyoleanolic | | |
| | acid) | | |
| NP-012672 | 16alpha,23- | Ara/Xyl-(1→4)-Rha/Fuc- | Ara/Xyl- |
| | Dihydroxyoleanolic | (1→2)-Glc/Gal-(1→2)- | |
| | acid | Rha/Fuc-(1→2)-GlcA- | |
| NP-017777 | Gypsogenin | Gal-(1→2)-[Xyl-(1→3)]- | Xyl-(1→4)-Rha-(1→2)-[R-(→4)]-Fuc- (R = |
| | | GlcA- | 4E-Methoxycinnamic acid) |
| NP-017778 | Gypsogenin | Gal-(1→2)-[Xyl-(1→3)]- | Xyl-(1→4)-Rha-(1→2)-[R-(→4)]-Fuc- (R = |
| | | GlcA- | 4Z-Methoxycinnamic acid) |
| NP-017774 | Gypsogenin | Gal-(1→2)-[Xyl-(1→3)]- | Xyl-(1→4)-[Gal-(1→3)]-Rha-(1→2)-4-OAc- |
| | | GlcA- | Fuc- |
| NP-018110^{c}, NP- | Gypsogenin | Gal-(1→2)-[Xyl-(1→3)]- | Xyl-(1→4)-[Glc-(1→3)]-Rha-(1→2)-3,4-di- |
| 017772^{d} | | GlcA- | OAc-Fuc- |
| NP-018109 | Gypsogenin | Gal-(1→2)-[Xyl-(1→3)]- | Xyl-(1→4)-[Glc-(1→3)]-Rha-(1→2)-[R- |
| | | GlcA | (→4)]-3-OAc-Fuc- (R = 4E- |
| | | | Methoxycinnamic acid) |
| NP-017888 | Gypsogenin | Gal-(1→2)-[Xyl-(1→3)]- | Glc-(1→3)-Xyl-(1→4)-[Glc-(1→3)]-Rha- |
| | | GlcA- | (1→2)-4-OAc-Fuc- |
| NP-017889 | Gypsogenin | Gal-(1→2)-[Xyl-(1→3)]- | Glc-(1→3)-Xyl-(1→4)-Rha-(1→2)-4-OAc- |
| | | GlcA- | Fuc- |
| NP-018108 | Gypsogenin | Gal-(1→2)-[Xyl-(1→3)]- | Ara/Xyl-(1→3)-Ara/Xyl-(1→4)-Rha/Fuc- |
| | | GlcA- | (1→2)-[4-OAc-Rha/Fuc-(1→4)]-Rha/Fuc- |
| SA1641^{a}, AE X55^{b} | Gypsogenin | Gal-(1→2)-[Xyl-(1→3)]- | Xyl-(1→3)-Xyl-(1→4)-Rha-(1→2)-[Qui- |
| | | GlcA- | (1→4)]-Fuc- |
| NP-017674 | Quillaic acid | Gal-(1→2)-[Xyl-(1→3)]- | Api-(1→3)-Xyl-(1→4)-[Glc-(1→3)]-Rha- |
| | | GlcA- | (1→2)-Fuc- |
| NP-017810 | Quillaic acid | Gal-(1→2)-[Xyl-(1→3)]- | Xyl-(1→4)-[Gal-(1→3)]-Rha-(1→2)-Fuc- |
| | | GlcA- | |
| AG1 | Quillaic acid | Gal-(1→2)-[Xyl-(1→3)]- | Xyl-(1→4)-[Glc-(1→3)]-Rha-(1→2)-Fuc- |
| | | GlcA- | |
| NP-003881 | Quillaic acid | Gal-(1→2)-[Xyl-(1→3)]- | Ara/Xyl-(1→4)-Rha/Fuc-(1→4)-[Glc/Gal- |
| | | GlcA- | (1→2)]-Fuc- |
| NP-017676 | Quillaic acid | Gal-(1→2)-[Xyl-(1→3)]- | Api-(1→3)-Xyl-(1→4)-[Glc-(1→3)]-Rha- |
| | | GlcA- | (1→2)-[R-(→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| NP-017677 | Quillaic acid | Gal-(1→2)-[Xyl-(1→3)]- | Api-(1→3)-Xyl-(1→4)-Rha-(1→2)-[R-(→4)]- |
| | | GlcA- | Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| NP-017706 | Quillaic acid | Gal-(1→2)-[Xyl-(1→3)]- | Api-(1→3)-Xyl-(1→4)-Rha-(1→2)-[Rha- |
| | | GlcA- | (1→3)]-4-OAc-Fuc- |
| NP-017705 | Quillaic acid | Gal-(1→2)-[Xyl-(1→3)]- | Api-(1→3)-Xyl-(1→4)-[Glc-(1→3)]-Rha- |
| | | GlcA- | (1→2)-[Rha-(1→3)]-4-OAc-Fuc- |
| NP-017773 | Quillaic acid | Gal-(1→2)-[Xyl-(1→3)]- | 6-OAc-Glc-(1→3)-Xyl-(1→4)-Rha-(1→2)-[3- |
| | | GlcA- | OAc-Rha-(1→3)]-Fuc- |
| NP-017775 | Quillaic acid | Gal-(1→2)-[Xyl-(1→3)]- | Glc-(1→3)-Xyl-(1→4)-Rha-(1→2)-[3-OAc-- |
| | | GlcA- | Rha-(1→3)]-Fuc- |
| SA1657 | Quillaic acid | Gal-(1→2)-[Xyl-(1→3)]- | Xyl-(1→3)-Xyl-(1→4)-Rha-(1→2)-[Qui- |
| | | GlcA- | (1→4)]-Fuc- |
| AG2 | Quillaic acid | Gal-(1→2)-[Xyl-(1→3)]- | Glc-(1→3)-[Xyl-(1→4)]-Rha-(1→2)-[Qui- |
| | | GlcA- | (1→4)]-Fuc- |
| SO1861 | Quillaic acid | Gal-(1→2)-[Xyl-(1→3)]- | Glc-(1→3)-Xyl-(1→4)-Rha-(1→2)-[Xyl- |
| | | GlcA- | (1→3)-4-OAc-Qui-(1→4)]-Fuc- |
| GE1741 | Quillaic acid | Gal-(1→2)-[Xyl-(1→3)]- | Xyl-(1→3)-Xyl-(1→4)-Rha-(1→2)-[3,4-di- |
| | | GlcA- | OAc-Qui-(1→4)]-Fuc- |
| SO1542 | Quillaic acid | Gal-(1→2)-[Xyl-(1→3)]- | Glc-(1→3)-[Xyl-(1→4)]-Rha-(1→2)-Fuc- |
| | | GlcA- | |
| SO1584 | Quillaic acid | Gal-(1→2)-[Xyl-(1→3)]- | 6-OAc-Glc-(1→3)-[Xyl-(1→4)]-Rha-(1→2)- |
| | | GlcA- | Fuc- |
| SO1658 | Gypsogenin | Gal-(1→2)-[Xyl-(1→3)]- | Glc-(1→3)-[Xyl-(1→3)-Xyl-(1→4)]-Rha- |
| | | GlcA- | (1→2)-Fuc- |
| SO1674 | Quillaic acid | Gal-(1→2)-[Xyl-(1→3)]- | Glc-(1→3)-[Xyl-(1→3)-Xyl-(1→4)]-Rha- |
| | | GlcA- | (1→2)-Fuc- |
| SO1832 | Quillaic acid | Gal-(1→2)-[Xyl-(1→3)]- | Xyl-(1→3)-Xyl-(1→4)-Rha-(1→2)-[Xyl- |
| | | GlcA- | (1→3)-4-OAc-Qui-(1→4)]-Fuc- |
| QS-7 (also referred | Quillaic acid | Gal-(1→2)-[Xyl-(1→3)]- | Api/Xyl-(1→3)-Xyl-(1→4)-[Glc-(1→3)]-Rha- |
| to as QS1861) | | GlcA- | (1→2)-[Rha-(1→3)]-40Ac-Fuc- |
| QS-7 api (also | Quillaic acid | Gal-(1→2)-[Xyl-(1→3)]- | Api-(1→3)-Xyl-(1→4)-[Glc-(1→3)]-Rha- |
| referred to as | | GlcA- | (1→2)-[Rha-(1→3)]-40Ac-Fuc- |
| QS1862) | | | |
| QS-17 | Quillaic acid | Gal-(1→2)-[Xyl-(1→3)]- | Api/Xyl-(1→3)-Xyl-(1→4)-[Glc-(1→3)]-Rha- |
| | | GlcA- | (1→2)-[R-(→4)]-Fuc- |
| | | | (R = 5-O-[5-O-Rha-(1→2)-Ara/Api-3,5- |
| | | | dihydroxy-6-methyl-octanoyl]-3,5- |
| | | | dihydroxy-6-methyl-octanoic acid) |
| QS-18 | Quillaic acid | Gal-(1→2)-[Xyl-(1→3)]- | Api/Xyl-(1→3)-Xyl-(1→4)-[Glc-(1→3)]-Rha- |
| | | GlcA- | (1→2)-[R-(→4)]-Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 A-apio | Quillaic acid | Gal-(1→2)-[Xyl-(1→3)]- | Api-(1→3)-Xyl-(1→4)-Rha-(1→2)-[R-(→4)]- |
| | | GlcA- | Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 A-xylo | Quillaic acid | Gal-(1→2)-[Xyl-(1→3)]- | Xyl-(1→3)-Xyl-(1→4)-Rha-(1→2)-[R-(→4)]- |
| | | GlcA- | Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 B-apio | Quillaic acid | Gal-(1→2)-[Xyl-(1→3)]- | Api-(1→3)-Xyl-(1→4)-Rha-(1→2)-[R-(→3)]- |
| | | GlcA- | Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| QS-21 B-xylo | Quillaic acid | Gal-(1→2)-[Xyl-(1→3)]- | Xyl-(1→3)-Xyl-(1→4)-Rha-(1→2)-[R-(→3)]- |
| | | GlcA- | Fuc- |
| | | | (R = 5-O-[5-O-Ara/Api-3,5-dihydroxy-6- |
| | | | methyl-octanoyl]-3,5-dihydroxy-6-methyl- |
| | | | octanoic acid) |
| beta-Aescin | Protoaescigenin- | Glc-(1→2)-[Glc-(1→4)]- | — |
| (described: Aescin | 21(2-methylbut-2- | GlcA- | |
| Ia) | enoate)-22-acetat | Glc-(1→2)-Ara-(1→3)-[Gal- | |
| Teaseed saponin I | 23-Oxo- | (1→2)]-GlcA- | — |
| | barringtogenol C- | | |
| | 21,22-bis(2-methylbut-2-enoate) | | |
| Teaseedsaponin J | 23-Oxo- | Xyl-(1→2)-Ara-(1→3)-[Gal- | — |
| | barringtogenol C- | (1→2)]-GlcA- | |
| | 21,22-bis(2-methylbut-2-enoate) | | |
| Assamsaponin F | 23-Oxo- | Glc-(1→2)-Ara-(1→3)-[Gal- | — |
| | barringtogenol C- | (1→2)]-GlcA- | |
| | 21(2-methylbut-2-enoate)-16,22- | | |
| | diacetat | | |
| Digitonin | Digitogenin | Glc-(1→3)-Gal-(1→2)-[Xyl- | — |
| | | (1→3)]-Glc-(1→4)-Gal- | |
| Primula acid 1 | 3,16,28- | Rha-(1→2)-Gal-(1→3)-[Glc- | — |
| | Trihydroxyoleanan- | (1→2)]-GlcA- | |
| | 12-en | | |
| AS64R | Gypsogenic acid | — | Glc-(1→3)-[Glc-(1→6)]-Gal- |
| | | Carbohydrate substituent | |
| | | at the C-23-OH group | |
| AS6.2 | Gypsogenic acid | Gal- | Glc-(1→3)-[Glc-(1→6)]-Gal- |
| Sapofectosid¹ | Quillaic acid | | |
| Agrostemmoside E | Quillaic acid | | |
| (AG1856, AG2.8)² | | | |

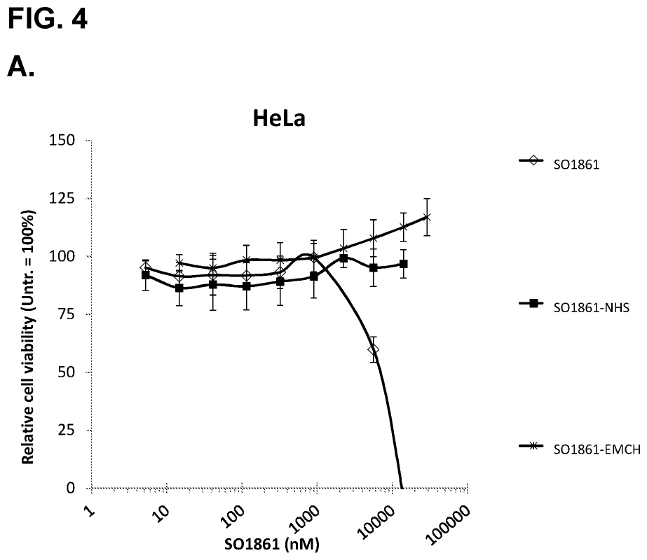

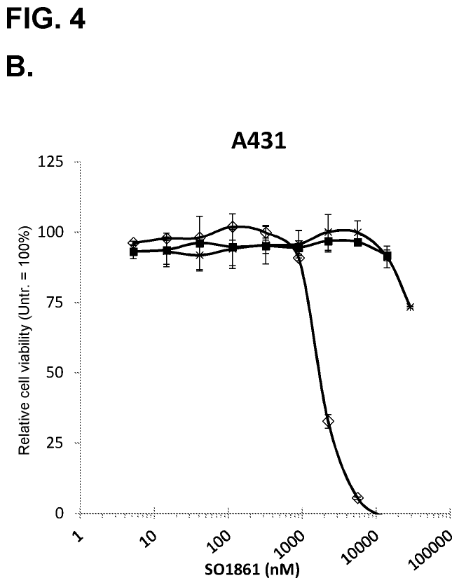

^{a,b}Different names refer to different isolates of the same structure
^{c,d}Different names refer to different isolates of the same structure
¹The structure of sapofectosid is provided in S. Sama et al, Sapofectosid - Ensuring non-toxic and effective DNA and RNA delivery, International Journal of Pharmaceutics, Volume 534, Issues 1-2, 20 Dec. 2017, Pages 195-205.
²The structure of Agrostemmoside E (also referred to as AG1856 or AG2.8) is given in FIG. 4 of J. Clochard et al, A new acetylated triterpene saponin from Agrostemma githago L. modulates gene delivery efficiently and shows a high cellular tolerance, International Journal of Pharmaceutics, Volume 589, 15 Nov. 2020, 119822.

An example of a saponin suitable for saponin derivative synthesis and for application of the obtained saponin derivative in the conjugate of the invention is a mono-desmosidic or bi-desmosidic triterpene saponin belonging to the type of a 12,13-dehydrooleanane with the aldehyde group in position C-23 and optionally comprising a glucuronic acid group in a carbohydrate substituent at the C-3beta-OH group of the saponin, preferably a bi-desmosidic triterpene saponin belonging to the type of a 12,13-dehydrooleanane with the aldehyde group in position C-23 and comprising a glucuronic acid group in a carbohydrate substituent at the C-3beta-OH group of the saponin. An exemplary saponin according to the invention comprises one, several, or all of the features of the saponin depicted as SAPONIN A and illustrated by the following structure ‘SAPONIN A’:

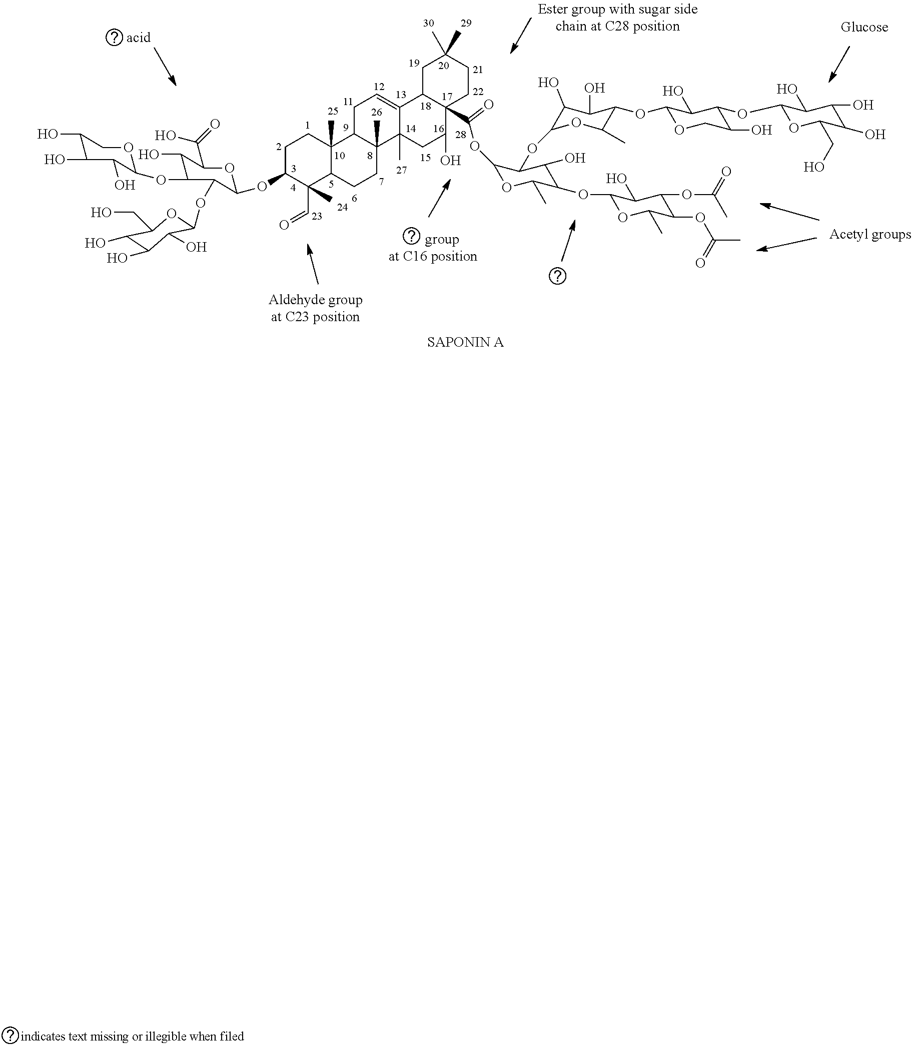

This group of saponins has demonstrated endosomal escape enhancing activity towards an effector moiety when the saponin and the effector moiety were present in the endosome of a cell. Typically, the saponins suitable for providing saponin derivatives of the invention and for application in the conjugates according to the invention, once derivatised, are saponins with a triterpene backbone wherein the structure of the triterpene backbone is a pentacyclic C30 terpene skeleton (also referred to as sapogenin or aglycone).

TABLE A2
Tumor-specific cell-surface receptor targets which can
be targeted by a cell-surface molecule targeting (binding)
molecule such as immunoglobulins, and antibodies that
can be used for the saponin conjugates of the invention,
ADC- and AOC conjugates comprised by pharmaceutical compositions
or pharmaceutical combinations of the invention (not
presented as a limitation; further immunoglobulins are
equally suitable for the invention)

| Target cell- | |
|---|---|
| surface | |
| receptor | Example monoclonal antibodies |
| HER2 | anti-HER2 monoclonal antibody such as trastuzumab and |
| | pertuzumab |
| CD20 | anti-CD20 monoclonal antibody such as rituximab, |
| | ofatumumab, tositumomab and ibritumomab |
| CA125 | anti-CA125 monoclonal antibody such as oregovomab |
| EpCAM | anti-EpCAM (17-1A) monoclonal antibody such as |
| (17-1A) | edrecolomab |
| EGFR | anti-EGFR monoclonal antibody such as cetuximab, |
| | panitumumab and nimotuzumab |
| CD30 | anti-CD30 monoclonal antibody such brentuximab |
| CD33 | anti-CD33 monoclonal antibody such as gemtuzumab and |
| | huMy9-6 |
| vascular | anti-vascular integrin alpha-v beta-3 monoclonal antibody |
| integrin | such as etaracizumab |
| alpha-v | |
| beta-3 | |
| CD52 | anti-CD52 monoclonal antibody such as alemtuzumab |
| CD22 | anti-CD22 monoclonal antibody such as epratuzumab |
| CEA | anti-CEA monoclonal antibody such as labetuzumab |
| CD44v6 | anti-CD44v6 monoclonal antibody such as bivatuzumab |
| FAP | anti- FAP monoclonal antibody such as sibrotuzumab |
| CD19 | anti-CD19 monoclonal antibody such as huB4 |
| CanAg | anti-CanAg monoclonal antibody such as huC242 |
| CD56 | anti-CD56 monoclonal antibody such huN901 |
| CD38 | anti-CD38 monoclonal antibody such as daratumumab |
| CA6 | anti-CA6 monoclonal antibody such as DS6 |
| IGF-IR | anti-IGF-IR monoclonal antibody such as cixutumumab |
| | and 3B7 |
| integrin | anti-integrin monoclonal antibody such as CNTO 95 |
| syndecan-1 | anti-syndecan-1 monoclonal antibody such as B-B4 |

The invention is further illustrated by the following examples, which should not be interpreted as limiting the present invention in any way.

### EXAMPLES

### Abbreviations

BOP (Benzotriazol-1-yloxy)tris(dimethylamino)phosphonium hexafluorophosphate
DIPEA N,N-diisopropylethylamine
DMF N,N-dimethylformamide
EDCI·HCl 3-((Ethylimino)methyleneamino)-N,N-dimethylpropan-1-aminium chloride
EMCH·TFA N-(c-maleimidocaproic acid) hydrazide, trifluoroacetic acid salt
mab monoclonal antibody
min minutes
NMM 4-Methylmorpholine
r.t. retention timeSPT1 or SPT001 saponin SO1861
TCEP tris(2-carboxyethyl)phosphine hydrochloride
Temp temperature
TFA trifluoroacetic acid
Trast Trastuzumab

Analytical Methods

LC-MS method 1,¹

Apparatus: Waters IClass; Bin. Pump: UPIBSM, SM: UPISMFTN with SO; UPCMA, PDA: UPPDATC, 210-320 nm, SQD: ACQ-SQD2 ESI, pos/neg 100-800; ELSD: gas pressure 40 psi, drift tube temp: 50° C.; column: Acquity C18, 50×2.1 mm, 1.7 μm Temp: 60° C., Flow: 0.6 mL/min, Gradient: to =5% B, t=98% B, t2.7 min=98% B, Post time: 0.3 min, Eluent A: 0.1% formic acid in water, Eluent B: 0.1% formic acid in acetonitrile.

LC-MS method 1,²

Apparatus: Waters IClass; Bin. Pump: UPIBSM, SM: UPISMFTN with SO; UPCMA, PDA: UPPDATC, 210-320 nm, SQD: ACQ-SQD2 ESI, neg 2000-3000; ELSD: gas pressure 40 psi, drift tube temp: 50° C.; column: Acquity C18, 50×2.1 mm, 1.7 μm Temp: 60° C., Flow: 0.6 mL/min, Gradient: t₀=5% B, t_{5.0min}=98% B, t_{6.0min}=98% B, Post time: 1.0 min, Eluent A: 0.1% formic acid in water, Eluent B: 0.1% formic acid in acetonitrile.

LC-MS method 1,³

Apparatus: Waters IClass; Bin. Pump: UPIBSM, SM: UPISMFTN with SO; UPCMA, PDA: UPPDATC, 210-320 nm, SQD: ACQ-SQD2 ESI, neg/pos 1500-2400; ELSD: gas pressure 40 psi, drift tube temp: 50° C.; column: Acquity C18, 50×2.1 mm, 1.7 μm Temp: 60° C., Flow: 0.6 mL/min, lin. gradient depending on the polarity of the product: t₀=2% B, t_{5.0min}=98% B, t_{6.0min}=98% B, Post time: 1.0 min, Eluent A: 10 mM ammonium bicarbonate in water (pH=9.5), Eluent B: acetonitrile.

LC-MS method 1,⁴

Apparatus: Waters IClass; Bin. Pump: UPIBSM, SM: UPISMFTN with SO; UPCMA, PDA: UPPDATC, 210-320 nm, SQD: ACQ-SQD2 ESI, neg/pos 1500-2400; ELSD: gas pressure 40 psi, drift tube temp: 50° C.; column: Acquity C18, 50×2.1 mm, 1.7 μm Temp: 60° C., Flow: 0.6 mL/min, lin. gradient depending on the polarity of the product: t₀=2% B, t_{5.0min}=50% B, t_{6.0min}=98% B, Post time: 1.0 min, Eluent A: 10 mM ammonium bicarbonate in water (pH=9.5), Eluent B: acetonitrile.

Preparative Methods

Preparative MP-LC method 1,¹

Instrument type: Reveleris™ prep MPLC; Column: Phenomenex LUNA C18(3) (150×25 mm, 10 μm); Flow: 40 mL/min; Column temp: room temperature; Eluent A: 0.1% (v/v) Formic acid in water, Eluent B: 0.1% (v/v) Formic acid in acetonitrile; Gradient: t₀ₘᵢₙ=5% B, t₁ₘᵢₙ=5% B, t₂ₘᵢₙ=20% B, t₁₇ₘᵢₙ=60% B, t₁₈ₘᵢₙ=100% B, t₂₃ₘᵢₙ=100% B; Detection UV: 210, 235, 254 nm and ELSD.

Preparative MP-LC method 2,²

Instrument type: Reveleris™ prep MPLC; column: Waters XSelect™ CSH C18 (145×25 mm, 10 μm); Flow: 40 mL/min; Column temp: room temperature; Eluent A: 10 mM ammoniumbicarbonate in water pH=9.0); Eluent B: 99% acetonitrile+1% 10 mM ammoniumbicarbonate in water; Gradient: t₀ₘᵢₙ=5% B, t₁ₘᵢₙ=5% B, t₂ₘᵢₙ=10% B, t₁₇ₘᵢₙ=50% B, t₁₈ₘᵢₙ=100% B, t₂₃ₘᵢₙ=100% B; Detection UV: 210, 235, 254 nm and ELSD.

Flash Chromatography

Grace Reveleris X2® C-815 Flash; Solvent delivery system: 3-piston pump with auto-priming, 4 independent channels with up to 4 solvents in a single run, auto-switches lines when solvent depletes; maximum pump flow rate 250 mL/min; maximum pressure 50bar (725 psi); Detection: UV 200-400 nm, combination of up to 4 UV signals and scan of entire UV range, ELSD; Column sizes: 4-330 g on instrument, luer type, 750 g up to 3000 g with optional holder.

UV-vis spectrophotometry

Protein concentrations were determined using a Thermo Nanodrop 2000 spectrometer or Lambda Spectrophotometer.

Size Exclusion Chromatography (SEC)

The conjugates were analysed by SEC using an Akta purifier 10 system and Biosep SEC-s3000 column eluting with DPBS:IPA (85:15). Conjugate purity was determined by integration of the Conjugate peak with respect to aggregate forms.

SDS-PAGE

Native proteins and conjugates were analysed under heat denaturing non-reducing and reducing conditions by SDS-PAGE against a protein ladder using a 4-12% bis-TRIS gel and MOPS as running buffer (200 V, ˜50 minutes). Samples were prepared to 0.5 mg/ml, comprising LDS sample buffer and MOPS running buffer as diluent. For reducing samples, DTT was added to a final concentration of 50 mM. Samples were heat treated for 2 minutes at 90-95° C. and 5 μg (10 μl) added to each well. Protein ladder (10 μl) was loaded without pre-treatment. Empty lines were filled with 1× LDS sample buffer (10 μl). After the gel was run, it was washed thrice with DI water (100 ml) with shaking (15 minutes, 200 rpm). Coomassie staining was performed by shaker-incubating the gel with PAGEBlue protein stain (30 ml) (60 minutes, 200 rpm). Excess staining solution was removed, rinsed twice with DI water (100 ml) and destained with DI water (100 ml) (60 minutes, 200 rpm). The resulting gel was imaged and processed using imageJ.

Western Blotting (WB)

From SDS-PAGE, the gel was transferred to nitrocellulose membrane using the X-Cell blot module with the following setup (BP-BP-FP-Gel-NC-FP-BP-FP-Gel-NC-FP-BP-BP) and conditions (30 V, 60 minutes) using freshly prepared transfer buffer. BP—blotting pad; FP—Filter pad; NC—Nitrocellulose membrane. After, the NC were washed thrice with PBS-T (100 ml) with shaking (5 minutes, 100 rpm), non-specific sites blocked with blocking buffer (30 ml) with shaking (10 minutes, 200 rpm) then active sites labelled with a combination of Goat anti-Human Kappa—HRP (1:2000) and Goat anti-Human IgG—HRP (1:2000) (30 ml) diluted in blocking buffer with shaking (60 minutes, 200 rpm). After, the NC was washed once with PBS-T (100 ml) with shaking (5 minutes, 100 rpm) and complexed antibody detected with freshly prepared, freshly filtered CN/DAB substrate (25 ml). Colour development was observed visually, and after 2-3 minutes development was stopped by washing the NC with water, and the resulting Blot photographed.

Materials

Trastuzumab (Herceptin®, Roche), cetuximab (Erbitux®, Merck KGaA) were purchased from the pharmacy (Charite, Berlin). CD71 monoclonal antibody was purchased from BioCell (Okt9, #BE0023). SO1861 was isolated and purified by Analyticon Discovery GmbH from raw plant extract obtained from Saponaria officinalis L. EGFdianthin was produced from E. coli according to standard procedures. Tris(2-carboxyethyl)phosphine hydrochloride (TCEP, 98%, Sigma-Aldrich), 5,5-Dithiobis(2-nitrobenzoic acid) (DTNB, Ellman's reagent, 99%, Sigma-Aldrich), Zeba™ Spin Desalting Columns (2 mL, Thermo-Fisher), NuPAGE™ 4-12% Bis-Tris Protein Gels (Thermo-Fisher), NuPAGE™ MES SDS Running Buffer (Thermo-Fisher), Novex™ Sharp Pre-stained Protein Standard (Thermo-Fisher), PageBlue™ Protein Staining Solution (Thermo-Fischer), Pierce™ BCA Protein Assay Kit (Thermo-Fisher), N-Ethylmaleimide (NEM, 98%, Sigma-Aldrich), 1,4-Dithiothreitol (DTT, 98%, Sigma-Aldrich), Sephadex G25 (GE Healthcare), Sephadex G50 M (GE Healthcare), Superdex 200P (GE Healthcare), Isopropyl alcohol (IPA, 99.6%, VWR), Tris(hydroxymethyl)aminomethane (Tris, 99%, Sigma-Aldrich), Tris(hydroxymethyl)aminomethane hydrochloride (Tris.HCL, Sigma-Aldrich), L-Histidine (99%, Sigma-Aldrich), D-(+)-Trehalose dehydrate (99%, Sigma-Aldrich), Polyethylene glycol sorbitan monolaurate (TWEEN 20, Sigma-Aldrich), Dulbecco's Phosphate-Buffered Saline (DPBS, Thermo-Fisher), Guanidine hydrochloride (99%, Sigma-Aldrich), Ethylenediaminetetraacetic acid disodium salt dihydrate (EDTA-Naz, 99%, Sigma-Aldrich), sterile filters 0.2 μm and 0.45 μm (Sartorius), Succinimidyl 4-(N-maleimidomethyl)cyclohexane-1-carboxylate (SMCC, Thermo-Fisher), Vivaspin T4 and T15 concentrator (Sartorius), Superdex 200PG (GE Healthcare), Tetra(ethylene glycol) succinimidyl 3-(2-pyridyldithio) propionate (PEG4-SPDP, Thermo-Fisher), [0-(7-Azabenzotriazol-1-yl)-N,N,N,N-tetramethyluronium-hexafluorphosphat] (HATU, 97%, Sigma-Aldrich), Dimethyl sulfoxide (DMSO, 99%, Sigma-Aldrich), N-(2-Aminoethyl)maleimide trifluoroacetate salt (AEM, 98%, Sigma-Aldrich), L-Cysteine (98.5%, Sigma-Aldrich), deionized water (DI) was freshly taken from Ultrapure Lab Water Systems (MilliQ, Merck), Nickel-nitrilotriacetic acid agarose (Ni-NTA agarose, Protino), Glycine (99.5%, VWR), 5,5-Dithiobis(2-nitrobenzoic acid (Ellman's reagent, DTNB, 98%, Sigma-Aldrich), S-Acetylmercaptosuccinic anhydride Fluorescein (SAMSA reagent, Invitrogen) Sodium bicarbonate (99.7%, Sigma-Aldrich), Sodium carbonate (99.9%, Sigma-Aldrich), PD MiniTrap desalting columns with Sephadex G-25 resin (GE Healthcare), PD10 G25 desalting column (GE Healthcare), Zeba Spin Desalting Columns in 0.5, 2, 5, and 10 mL (Thermo-Fisher), Vivaspin Centrifugal Filters T4 10 kDa MWCO, T4 100 kDa MWCO, and T15 (Sartorius), Biosep s3000 aSEC column (Phenomenex), Vivacell Ultrafiltration Units 10 and 30 kDa MWCO (Sartorius), Nalgene Rapid-Flow filter (Thermo-Fisher). Goat anti-Human IgG— horseradish peroxidase (HRP) (Southern Biotech), Goat anti-Human Kappa— HRP (Southern Biotech), Dulbecco's Phosphate-Buffered Saline (DPBS, Thermo-Fisher), Guanidine hydrochloride (99%, Sigma-Aldrich), 100 mM Na Bicarbonate buffer pH 8.3, NuPAG MOPS SDS Running Buffer (Thermo-Fisher), Pierce LDS Sample Buffer, Non-Reducing (Thermo-Fisher), NuPAGE Transfer Buffer (Thermo-Fisher), Polyethylene glycol sorbitan monolaurate (TWEEN 20, Sigma-Aldrich), 1M HCl (VWR), Methanol (Thermo-Fisher), Tris-buffered saline (TBS) Blocking Buffer (Thermo-Fisher), Dimethyl sulfoxide (DMSO, 99%, Sigma), 1,4-Dithiothreitol (DTT, 98%, Sigma), NuPAG Transfer Buffer (Thermo-Fisher), Isopropylalkohol (ISA, 98%, Sigma), Glycin (99%, Sigma), Vivaspin Centrifugal Filters T4 kDa MWCO, T4 100 kDa MWCO, and T15 kDa MWCO (Sartorius), Minisart 0.45 μm filter (Sartorius), Minisart 0.2 μm filter (Sartorius), PD10 G25 desalting column (GE Healthcare), Desalting column with Sephadex G-50M resin (1.6×35 cm, GE Healthcare), Pierce BCA Protein Assay Kit (Thermo-Fisher), Pierce™ Bovine Gamma Globulin Standard Ampules, 2 mg/mL (Thermo-Fisher), TNBSA Solution (2,4,6-trinitrobenzene sulfonic acid) (5% w/v) (Thermo-Fisher), Sodium dodecyl sulfate (SDS, 99%, Sigma), Novex Sharp Pre-stained Protein Standard ladder (Thermo-Fisher), NuPAGE 4-12% Bis-Tris Protein Gels (Thermo-Fisher), Nitrocellulose/Filter Paper Sandwich, 0.2 μm (Thermo-Fisher), PageBlue Protein Staining Solution (Thermo-Fisher), Pierce CN/DAB Substrate Kit (Thermo-Fisher), BioSep™ 5 μm SEC-s3000 400 A, LC Guard Column 75×7.8 mm (Phenomenex), Ultrafree-CL Centrifugal Filter 0.22 μm pore size (Sigma). To SO1861 (121 mg, 0.065 mmol) and EMCH.TFA (110 mg, 0.325 mmol) was added methanol (extra dry, 3.00 mL) and TFA (0.020 mL, 0.260 mmol). The reaction mixture stirred at room temperature. After 1.5 hours the reaction mixture was subjected to preparative MP-LC. 1 Fractions corresponding to the product were immediately pooled together, frozen and lyophilized overnight to give the title compound (120 mg, 90%) as a white fluffy solid. Purity based on LC-MS 96%.
LRMS (m/z): 2069 [M−1]¹⁻
LC-MS r.t. (min): 1.08⁴

SO1861-L-azide synthesis (molecule 23); see FIG. 9 (saponin according to formula (IV))

Chemical Formula: C₉₄H₁₅₁N₅O₅₀, Exact Mass: 2149,94 SO1861-L-PEG4-azide or SO1861-L-N3 was synthesized from SO1861 (molecule 2 in FIG. 9) and molecule 22 (see FIG. 9; also referred to as: azido-PEG4-amide)amine/TFA), therewith providing molecule 23 (FIG. 9; (saponin according to formula (IV))).

SO1861-L-NHS synthesis (molecule 25, FIG. 10) (saponin according to formula (VIII)) Chemical Formula: C₁₁₇H₁₆₉N₇O₅₅, Exact Mass: 2552.06 SO1861-L-azide (7.71 mg, 3.58 μmol; molecule 23) and DBCO-NHS (2.88 mg, 7.17 μmol; molecule 24, FIG. 10)) were dissolved in dry DMF (0.50 mL). The reaction mixture was shaken for 1 min and left standing at room temperature. After 30 min the reaction mixture was added dropwise to diethyl ether (40 mL). After centrifugation (7800 RPM, 5 min) the supernatant was decanted and the pellet was resuspended in diethyl ether (20 mL) and centrifuged again. After decanting the supernatant the residue was dissolved in water/acetonitrile (3:1, v/v, 3 mL) and the resulting solution was directly frozen and lyophilized overnight to give the title compound (8.81 mg, 96%) as a white fluffy solid. Purity based on LC-MS 84%. Contains 14% of the hydrolysed NHS ester.
LRMS (m/z): 2551 [M−1]¹⁻
LC-MS r.t. (min): 2.76/2.78² (double peaks due to isomers)

Cell Viability Assay

Cell viability was determined by an MTS-assay, performed according to the manufacturer's instruction (CellTiter 96® AQueous One Solution Cell Proliferation Assay, Promega). Briefly, the MTS solution was diluted 20× in DMEM without phenol red (PAN-Biotech GmbH) supplemented with 10% FBS (PAN-Biotech GmbH). The cells were washed once with 200 μL PBS per well, after which 100 μL diluted MTS solution was added per well. The plate was incubated for approximately 20-30 minutes at 37° C. Subsequently, the optical density at 492 nm was measured on a Thermo Scientific Multiskan FC plate reader (Thermo Scientific). For quantification the background signal of ‘medium only’ wells was subtracted from all other wells, before the ratio of untreated/treated cells was calculated, by dividing the background corrected signal of untreated wells over the background corrected signal of the treated wells.

FACS Analysis

Cells were seeded in DMEM (PAN-Biotech GmbH) supplemented with 10% fetal calf serum (PAN-Biotech GmbH) and 1% penicillin/streptomycin (PAN-Biotech GmbH), at 500,000 c/plate in 10 cm dishes and incubated for 48 hrs (5% CO₂, 37° C.), until a confluency of 90% was reached. Next, the cells were trypsinized (TrypIE Express, Gibco Thermo Scientific) to single cells. 0.75×10⁶ cells were transferred to a 15 mL falcon tube and centrifuged (1,400 rpm, 3 min). The supernatant was discarded while leaving the cell pellet submerged. The pellet was dissociated by gentle tapping the falcon tube on a vortex shaker and the cells were washed with 4 mL cold PBS (Mg²⁺ and Ca²⁺ free, 2% FBS). After washing the cells were resuspended in 3 mL cold PBS (Mg²⁺ and Ca²⁺ free, 2% FBS) and divided equally over 3 round bottom FACS tubes (1 mL/tube). The cells were centrifuged again and resuspended in 200 μL cold PBS (Mg²⁺ and Ca²⁺ free, 2% FBS) or 200 μL antibody solution; containing 5 μL antibody in 195 μL cold PBS (Mg²⁺ and Ca²⁺ free, 2% FBS). APC Mouse IgG1, K APC anti-human EGFR (#352906, Biolegend) was used to stain the EGFR receptor. PE anti-human HER2 APC anti-human CD340 (erbB2/HER-2) (#324408 Biolegend) was used to stain the HER2 receptor, PE Mouse IgG2a, K Isotype Ctrl FC (#400212, Biolegend) was used as its matched isotype control. PE anti-human CD71 (#334106, Biolegend) was used to stain the CD71 receptor, PE Mouse IgG2a, K Isotype Ctrl FC (#400212, Biolegend) was used as its matched isotype control. Samples were incubated for 30 min at 4° C. on a tube roller mixer. Afterwards, the cells were washed 3× with cold PBS (Mg²⁺ and Ca²⁺ free, 2% FBS) and fixated for 20 min at room temperature using a 2% PFA solution in PBS. Cells were washed 2× with cold PBS, and resuspended in 250-350 μL cold PBS for FACS analysis. Samples were analyzed with a BD FACSCanto II flow cytometry system (BD Biosciences) and FlowJo software. Results are displayed in Table 1.

TABLE 1
Expression levels of EGFR, HER2 and CD71 of various cells

| | EGFR | HER2 | CD71 |
|---|---|---|---|
| | expression | expression | expression |
| Cell line | level (MFI) | level (MFI) | level (MFI) |
| MDA-MB-468 | 1656 | 1 | 186 |
| A431 | 1593 | 10 | 322 |
| CaSki | 481 | 12 | 189 |
| SK-BR-3 | 28 | 1162 | 331 |
| JIMT-1 | 58 | 74 | 107 |
| HeLa | 91 | 7 | 312 |
| A2058 | 1 | 5 | 59 |

Hemolysis Assay

Red blood cells (RBCs) were isolated from a buffy coat using a Ficoll gradient. The obtained RBC pellet (→4-5 ml) was washed 2× with 50 ml DPBS (without Ca²⁺/Mg²⁺, PAN-Biotech GmbH). Cells were pelleted by centrifugation for 10 min, 800×g at RT. RBC were counted and resuspended at 500,000,000 c/ml in DPBS (without Ca²⁺/Mg²⁺), based on total cell count. SO1861-linker dilutions were prepared in DPBS (with Ca²⁺/Mg²⁺, PAN-Biotech GmbH), at 1.11× final strength. For positive lysis control a 0.02% Triton-X100 solution was prepared in DPBS^{+/+}. Of all compound solutions 135 μl was dispensed/well in a 96 well V-bottom plate. To this 15 μl RBC suspension was added and mixed shortly (10 sec-600 rpm). The plate was incubated 30 min at RT, with gentle agitation. Afterwards the plate was spun for 10 min at 800×g to pellet the RBC and 100-120 μl supernatant was transferred to a standard 96 wp. Subsequently, the OD at 405 nm was measured on a Thermo Scientific Multiskan FC plate reader (Thermo Scientific). For quantification the background signal of ‘DPBS^{+/+} only’ wells was subtracted from all other wells before the percentage of hemolysis was calculated in comparison to 0.02% Triton-X100, by dividing the background corrected signal of treated wells over the background corrected signal of the 0.02% Triton-X100 wells (×100).

CD71mab-Saporin conjugates

Custom CD71 mab-saporin conjugates were produced and purchased from Advanced Targeting Systems (San Diego, CA).

Antibody-Lys-(L-501861)₄ synthesis with a DAR4

Trastuzumab and Cetuximab are referred hereafter as “Ab”. Ab was conjugated to an NHS-conjugates SO1861-L-NHS linker via an active ester approach conducting an amide bond formation reaction between the lysine residues of the Ab and the NHS function site of SO1861-L-NHS. The procedure is exemplary described for Trastuzumab-Lys-(L-SO1861)₄:
To an aliquot of Trastuzumab (10.0 mg, 6.70×10⁻⁵ mmol, 2.50 mg/ml) was added an aliquot of freshly prepared SO1861-L-NHS solution (20.0 mg/ml, 4.0 mole equivalents, 26.8×10⁻⁵ mmol) in DMSO, the mixture homogenised by repeat pipetting and vortex mixing, then incubated for 60 minutes at 20° C. with roller-mixing. After, the reaction was quenched by the addition of an aliquot of a freshly prepared glycine solution (10.0 mg/ml, 5.0 mole equivalents, 134×10⁻⁵ mmol) in DPBS pH 7.5. The crude conjugate then purified by gel filtration using a 1.6×35 cm Sephadex G50M column eluting with DPBS pH 7.5. The product fraction was collected and pooled, then analysed by BCA colorimetric assay to ascertain antibody concentration. The product was then analysed by UV-vis spectrophotometry to ascertain a new mass ε280 for the conjugate (1.834 (mg/ml)⁻¹ cm⁻¹), concentrated by diafiltration using a vivaspin T4 centrifugal filter tube (3,000 g, 20° C., 5 minute intervals) to >2.50 mg/ml, spin-filtered to 0.2 μm then normalised to 2.50 mg/ml. The result was Tras-L-501861 (2.50 mg/ml, 2.84 ml, 71%, Tras:SO1861=0). The product was characterised by TNBS assay (SO1861 incorporation), aSEC (% purity), SDS-PAGE (apparent MW) and WB (anti-human antibody recognition). Results are displayed in Table 2.

TABLE 2
Summary of antibody-L-(NHS-SO1861)ⁿ conjugate characterization data

| | | | | Average | Average | |
|---|---|---|---|---|---|---|
| | | | | SO1861 | SO1861 | |
| | | | SO1861-L- | incorporation | incorporation | |
| | | | NHS molar | via | via | |
| | | | feed | electrophoresisª | colorimetry^{b} | |
| | | Yield | equivalents | (SO1861 to Ab | (SO1861 to Ab | Purity^{c} |
| batch | Conjugate | (%) | to Ab | ratio) | ratio) | (%) |
| DV265/22- | Trastuzumab | — | — | — | — | 100 |
| Tras | | | | | | |
| STB41/1-1 | Trastuzumab- | 71 | 4 | 8.9 | 0 | 98.2 |
| | Lys-L-SO1861 | | | | | |
| STB41/1-2 | Trastuzumab- | 66 | 6 | 9.5 | 1.5 | 96.6 |
| | Lys-L-SO1861 | | | | | |
| STB41/1-3 | Trastuzumab- | 56 | 8 | 9.5 | 4.0 | 96.6 |
| | Lys-L-SO1861 | | | | | |
| STB41/3-1 | Trastuzumab- | 72 | 4* | 10.2 | 25.4 | 99.8 |
| | Lys-L-SO1861 | | | | | |
| STB41/3-2 | Trastuzumab- | 73 | 6* | 9.5 | 26.9 | 99.8 |
| | Lys-L-SO1861 | | | | | |
| STB41/3-3 | Trastuzumab- | 64 | 8* | 9.5 | 28.9 | 99.6 |
| | Lys-L-SO1861 | | | | | |
| STB34/1-1 | Trastuzumab- | 81 | 8.6 | 5.4 | 15.9 | 99.4 |
| | Lys-L-SO1861 | | | | | |
| STB34/1-2 | Trastuzumab- | 54 | 16.8 | 8.7 | 23.3 | 99.1 |
| | Lys-L-SO1861 | | | | | |
| STB34/1-3 | Trastuzumab- | 11 | 33.3 | 9.2 | 25.6 | 98.3 |
| | Lys-L-SO1861 | | | | | |
| DV248/168- | Cetuximab | — | — | — | — | 100 |
| Cet | | | | | | |
| STB41/2-1 | Cetuximab-Lys- | 71 | 4 | 9.5 | 6.3 | 99.2 |
| | L-SO1861 | | | | | |
| STB41/2-2 | Cetuximab-Lys- | 72 | 6 | 6.8 | 9.2 | 98.6 |
| | L-SO1861 | | | | | |
| STB41/2-3 | Cetuximab-Lys- | 67 | 8 | 8.2 | 5.3 | 97.9 |
| | L-SO1861 | | | | | |
| STB34/2-1 | Cetuximab-Lys- | 99 | 9.4 | 5.4 | 35.2 | 99.1 |
| | L-SO1861 | | | | | |
| STB34/2-2 | Cetuximab-Lys- | 99 | 18.5 | 10.7 | 42.4 | 98.0 |
| | L-SO1861 | | | | | |
| STB34/2-3 | Cetuximab-Lys- | 66 | 33.4 | 9.1 | 42.6 | 97.2 |
| | L-SO1861 | | | | | |

^{a}SO1861 incorporation estimated from mass-shift of conjugate band with respect to native antibody on SDS-PAGE;
^{b}SPT001-L incorporations were estimated by colorimetric TNBS assay.
^{c}% purity with respect to aggregate by SEC.
*SO1861-L-NHS solution was added in a 2 mg/mL concentration, 10 x less than in all other batches.

Anti body-(EMCH-SO1861)ⁿ

Trastuzumab, Cetuximab, are referred hereafter as “Ab”. Ab was conjugated to SO18161-EMCH or via Michael-type thiol-ene conjugation reaction at DAR 4. The SO1861-EMCH molecule obtains a labile (L) pH sensitive bond between its structure and its maleimide function generating a labile bond between the SO1861 and Ab. The procedure is exemplary described for Cetuximab-(EMCH-SO1861)⁴: To a solution of Cetuximab (40 mg, 8.0 ml) was added 10 μl/ml each of Tris concentrate (127 mg/ml, 1.05 M), Tris.HCl concentrate (623 mg/ml, 3.95M) and EDTA-Naz concentrate (95 mg/ml, 0.26 M) to give a 50 mM TBS, 2.5 mM EDTA buffer pH 7.5. To Cetuximab divided into four portions (each of 9.73 mg, 4.864 mg/ml, 65 nmol) was added an aliquot of freshly prepared TCEP solution (0.5-2.0 mg/ml, 1.15-7.02 mole equivalents, 75-455 nmol), the mixtures vortexed briefly then incubated for 300 minutes at 20° C. with roller-mixing. After incubation (prior to addition of SO1861-EMCH), a ca. 1 mg (0.210 ml) aliquot of Ab-SH was removed from each mixture and purified by gel filtration using a zeba spin desalting column into TBS pH 7.5. These aliquots were characterized by UV-vis analysis and Ellman's assay. To each of the bulk Ab-SH was added an aliquot of freshly prepared SO1861-EMCH solution (2 mg/ml, 1.3 mole equivalents per ‘thiol’, 0.15-0.61 μmol, 0.16-0.63 ml), the mixtures vortexed briefly then incubated for 120 minutes at 20° C. Besides each conjugation reaction, two aliquots of desalted Ab-SH (0.25 mg, 1.67 nmol) were reacted with NEM (1.3 mole equivalents per ‘thiol’, 4.3-17.4 nmol, 2.2-8.7 μl of a 0.25 mg/ml solution) or TBS pH 7.5 buffer (2.2-8.7 μl) for 120 minutes at 20° C., as positive and negative controls, respectively. After incubation (prior to addition of NEM), a 0.200 ml aliquot of Ab-EMCH-SO1861 mixture was removed and purified by gel filtration using zeba spin desalting column into TBS pH 7.5. This aliquot was characterized by UV-vis and alongside positive and negative controls were characterized by Ellman's assay to obtain SO1861-EMCH incorporations. To the bulk Ab-EMCH-SO1861 mixture was added an aliquot of freshly prepared NEM solution (2.5 mg/ml, 2.5 -mole equivalents, 0.15-0.58 μmol) and the mixtures purified by zeba spin desalting columns eluting with DPBS pH 7.5 to give purified Cetuximab—(EMCH-SO1861) conjugates. The products were normalized to 2.5 mg/ml and filtered to 0.2 μm prior to dispensing for biological evaluation.

### Example 1

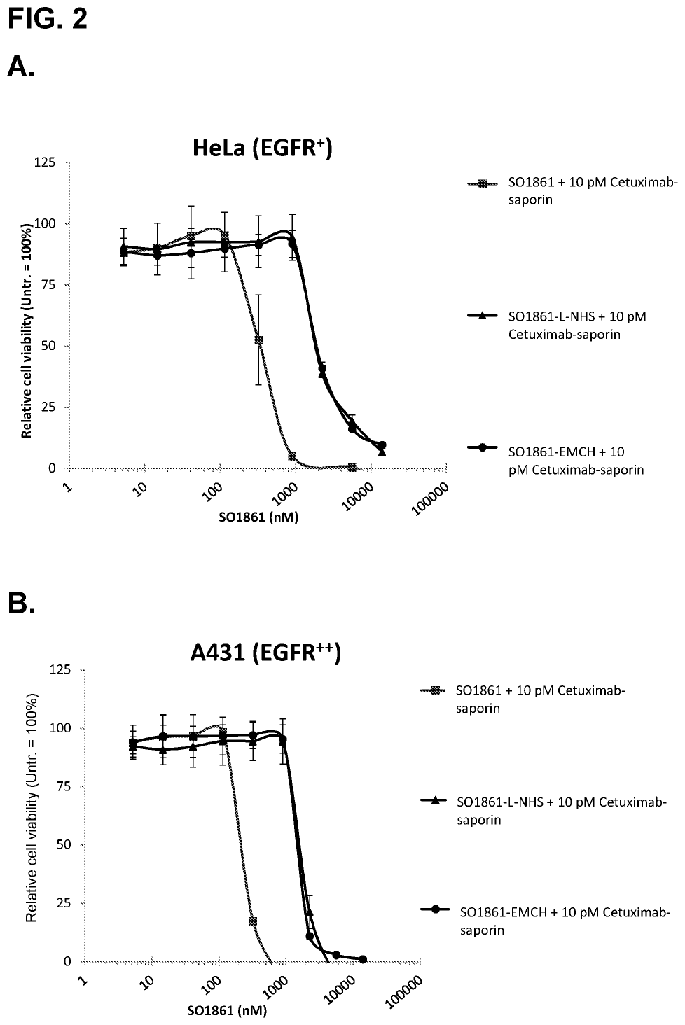

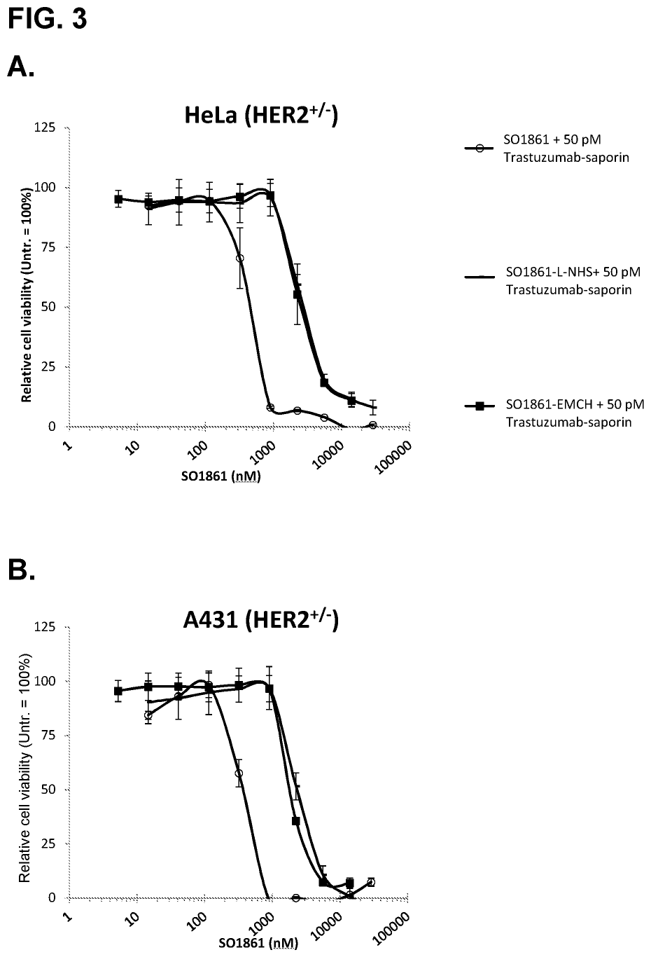

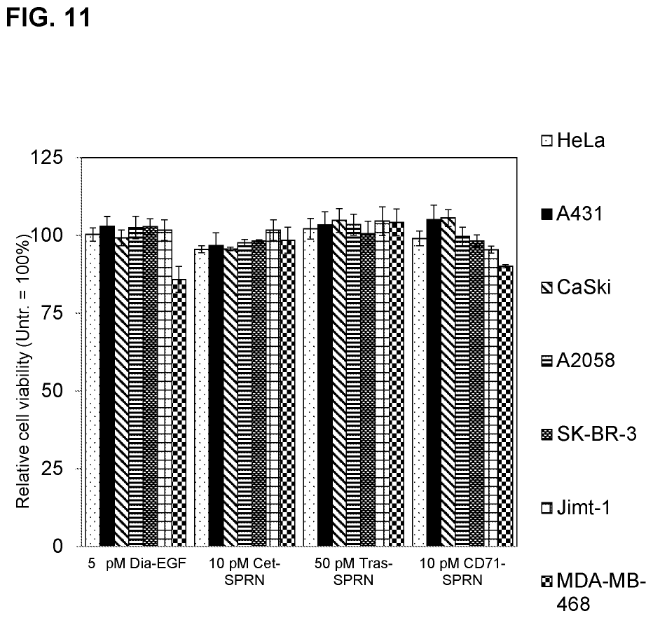

SO1861-L-NHS was tested for endosomal escape enhancing activity. For this, SO1861, SO1861-EMCH and SO1861-L-NHS were titrated in the presence of a non-effective fixed concentration (FIG. 11) of 5 pM EGFdianthin (10 pM EGFdianthin is shown in FIG. 11), 50 pM Trastuzumab-saporin or 10 pM Cetuximab-saporin on EGFR/HER2 expressing cells (HeLa and A431, Table 1). This revealed that SO1861-L-NHS combined with 5 pM EGFdianthin (1050=4000 nM (HeLa); IC50=3000 nM (A431); FIG. 1, Table 3, 4) or 10 pM Cetuximab-saporin (1050=2000 nM (HeLa); IC50=2000 nM (A431); FIG. 2) or 50 pM Trastuzumab-saporin (1050=3000 nM (HeLa); 1050=3000 nM (A431); FIG. 3) showed similar activity compared to SO1861-EMCH (FIG. 1, 2, 3). SO1861+5 pM EGFdianthin or 10 pM trastuzumab-saporin or 10 pM cetuximab-saporin showed strongest activity (FIG. 1, 2, 3).

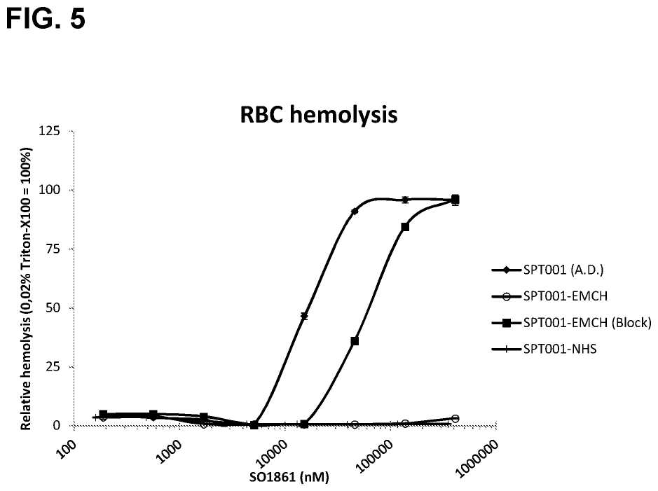

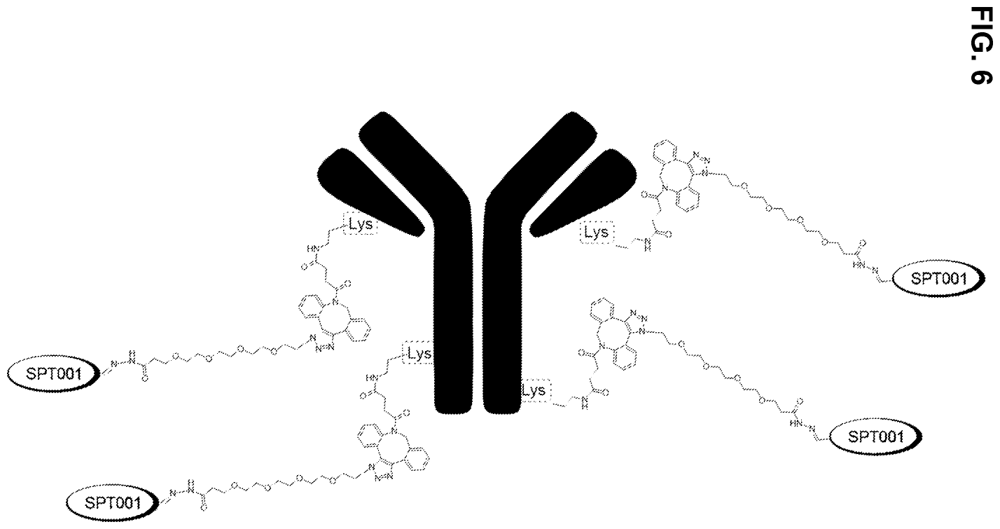

Next, toxicity was determined. For this, SO1861, SO1861-EMCH and SO1861-L-NHS were titrated on HeLa and A431 cells. This revealed that SO1861-L-NHS showed toxicity at 1050 >100,000 nM in HeLa and IC50>30,000 in A431, comparable with the toxicity observed with SO1861-EMCH (FIG. 4, Table 3, 4). SO1861 showed strongest toxicity (FIG. 4, Table 3, 4). Next, hemolysis assay was performed, for this SO1861, SO1861-EMCH, SO1861-EMCH (blocked) and SO1861-L-NHS (molecule VIII) were tested on fresh Human Red blood cells and this revealed comparable hemolytic activity of SO1861-EMCH and SO1861-L-NHS (FIG. 5, Table 3, 4). SO1861-EMCH (blocked): the maleimide functional group is conjugated with mercapto-ethanol.

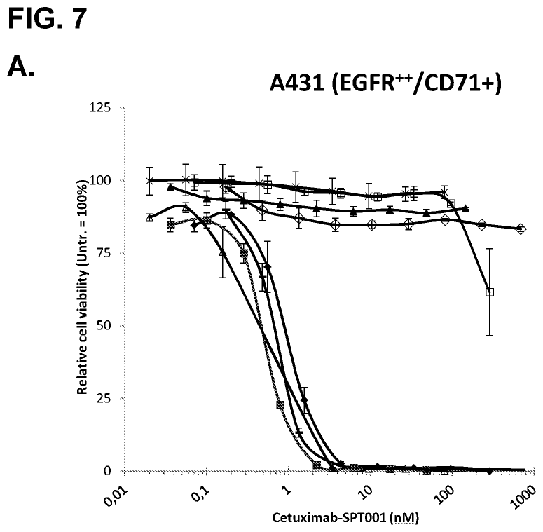

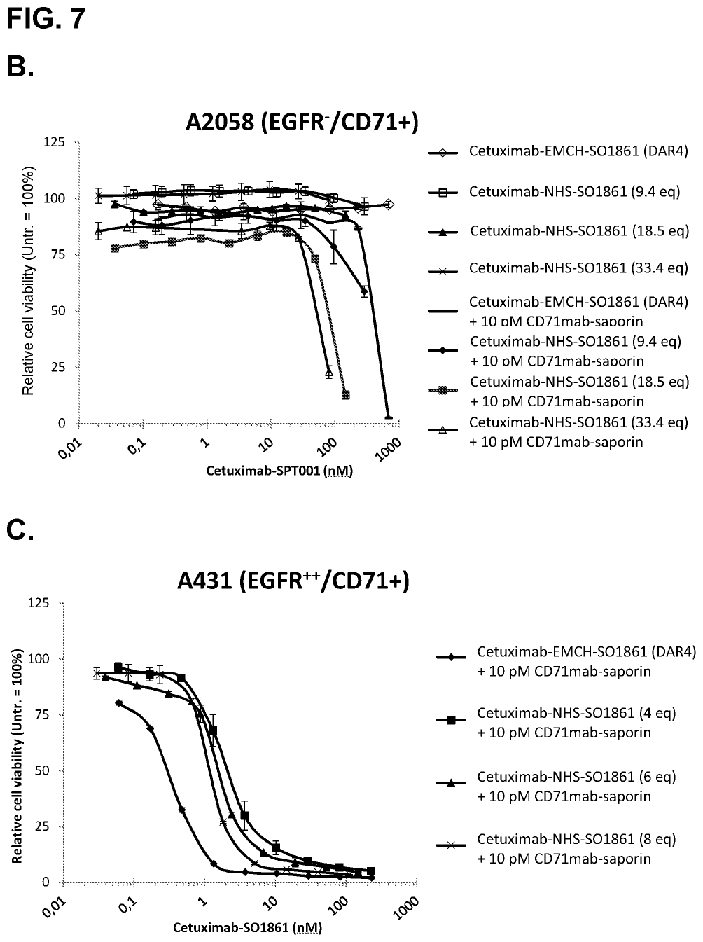

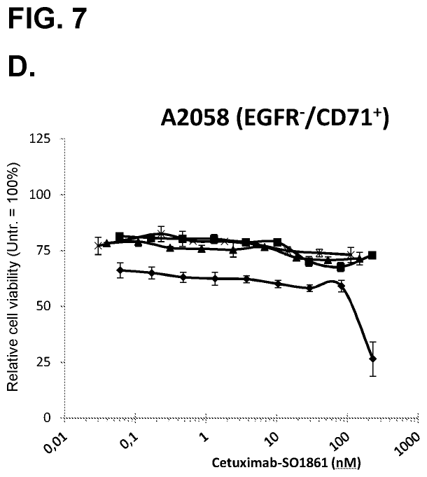

Next, SO1861-L-NHS was conjugated via lysine residues to cetuximab (monoclonal antibody recognizing and binding human EGFR, (4, 6, 8, 9.4, 18.5, 33.4 Mol equivalents, see Table 2) as depicted in FIG. 6. Cetuximab-(L-NHS-SO1861) was titrated on a fixed non-effective concentration of 10 pM CD71 mab-saporin (monoclonal antibody recognizing human CD71; OKT-9) (FIG. 11), conjugated to the protein toxin, saporin, and targeted protein-toxin mediated cell killing on EGFR++/CD71+(A431) and EGFRICD71+(A2058) expressing cells (Table 1) was determined. This revealed strong cell killing at low concentrations of Cetuximab-(L-NHS-SO1861) at 4, 6, 8, 9.4, 18.5 and 33.4 equivalents of L-NHS-SO1861 (A431: IC50 (9.4 eq; 18.5 eq; 33.4 eq)=0,5; 1050 (4 eq, 6 eq, 8 eq)=2 nM (FIG. 7A, 7C)). Compared to Cetuximab-(EMCH-SO1861) 4, the activity of Cetuximab-(L-NHS-SO1861)^{4 eq, 6 eq, 8 eq} was comparable whereas Cetuximab-(L-NHS-SO1861)^{9.4 eq, 18.5 eq, 33.4 eq} showed equal activity. Equivalent concentrations Cetuximab-(L-N HS-SO1861)^{9.4 eq, 18.5 eq, 33.4 eq} or Cetuximab-(EMCH-SO1861)⁴ could not induce any cell killing activity in EGFR⁺⁺/CD71⁺ expressing cells. Similar experiments in cells that lack EGFR expression (A2058; EGFR⁻/CD71⁺) revealed no activity at low nM Cetuximab-(L-NHS-SO1861)^{eq} in combination with 10 pM CD71 mab-saporin (IC50>50 nM; FIG. 7B, 7D).

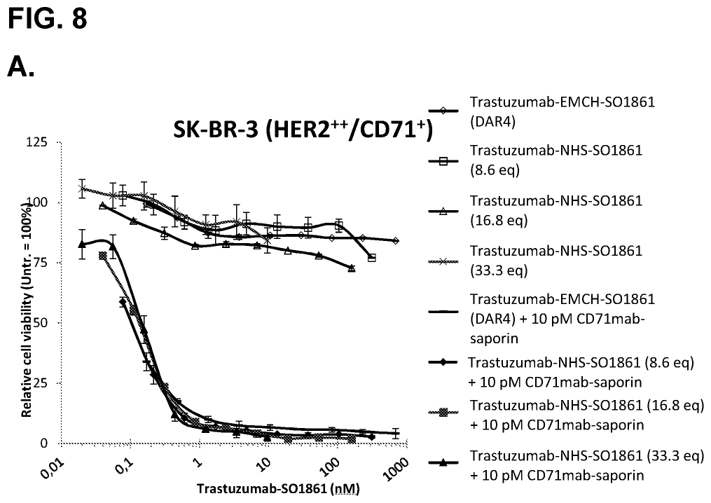

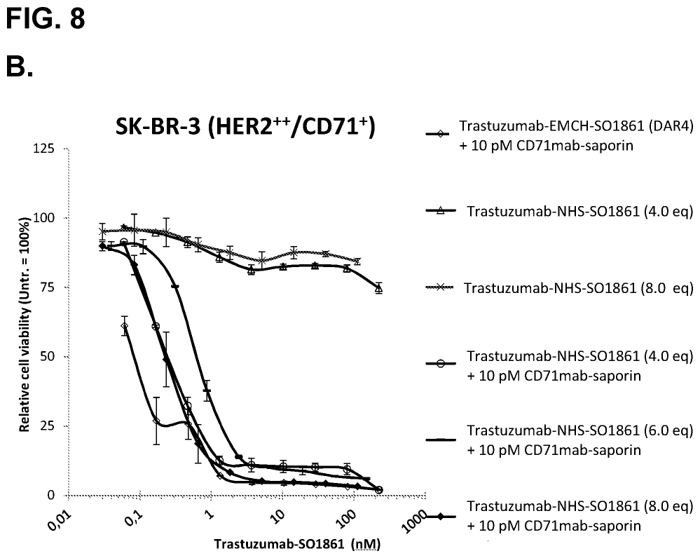

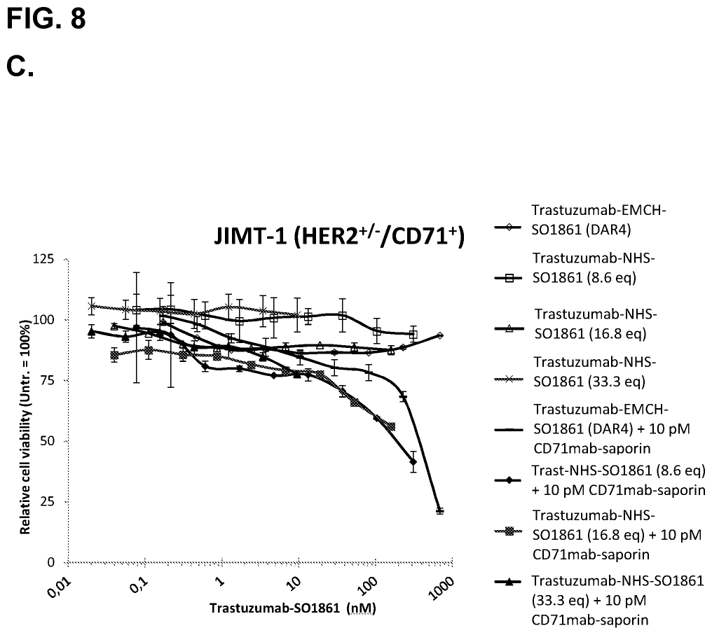

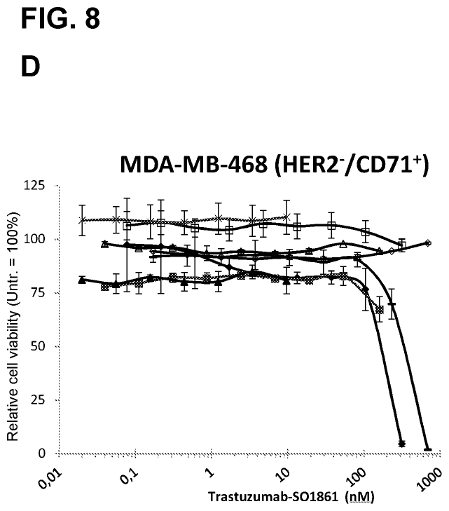

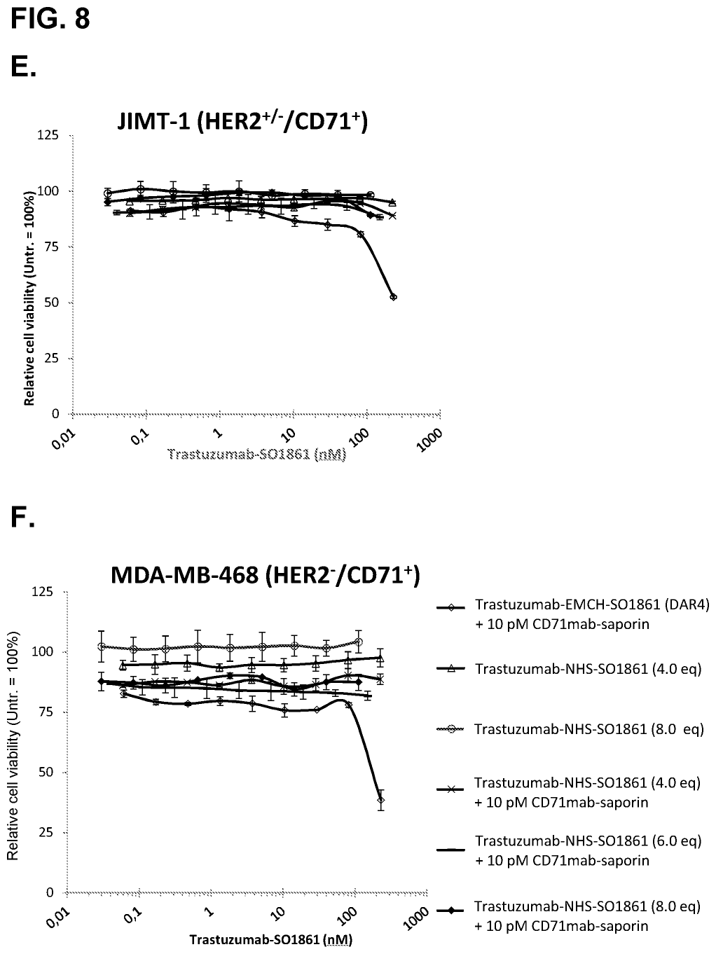

Next, SO1861-L-NHS was conjugated via lysine residues to Trastuzumab (monoclonal antibody recognizing and binding human HER2, (4, 6, 8, 8.6, 16.8, 33.4 Mole equivalents), Trastuzumab -(L-NHS-SO1861)^{eq} was titrated on a fixed non-effective concentration (FIG. 11) of 10 pM CD71 mab-saporin (monoclonal antibody recognizing human CD71; OKT-9), conjugated to the protein toxin, saporin, and targeted protein toxin mediated cell killing on HER2++/CD71+(A431), HER2^{+/-}/CD71⁺ (JIMT-1) and HER2⁻/CD71⁺ (MDA-MB-468) expressing cells (Table 1) was determined. This revealed strong cell killing at low concentrations of Trastuzumab-(L-NHS-SO1861) at 4, 6, 8, 8.6, 16.8, 33.4 equivalents of L-NHS-SO1861 (A431: IC50 (9.4 eq; 18.5 eq; 33.4 eq)=0,5; IC50 (4 eq, 6 eq, 8 eq)=2 nM (FIG. 8A, 8B)). Compared to Trastuzumab-(EMCH-SO1861)⁴, Trastuzumab-(L-NHS-SO1861)^{4 eq, 6 eq, 8 eq, 8.6 eq, 16.8 eq, 33.3 eq} showed equal activity. Equivalent concentrations Trastuzumab-(L-NHS-SO1861)^{4 eq, 8 eq 8.6 eq, 16.8 eq, 33.3 eq} or Trastuzumab-(EMCH-SO1861)⁴ could not induce any cell killing activity in HER2⁺⁺/CD71⁺ expressing cells. Similar experiments in cells that have low HER2 expression (JIMT-1; EGFR^{+/-}/CD71⁺) or lack HER2 expression (MDA-MB-468; EGFR⁻/CD71⁺) revealed only at high concentrations Trastuzumab-(L-NHS-SO1861)^{4 eq, 6 eq, 8 eq, 8.6 eq, 16.8 eq, 33.3 eq}, cell killing activity in combination with 10 pM CD71mab-saporin (IC50>100 nM; FIG. 8C-8E).

TABLE 3
IC50 overview of modified SO1861 activity (Hela cells)/toxicity (Hela cells)/hemolysis
(red blood cells)/CMC, for indicated SO1861 derivatives and SO1861.

| | | IC50 nM | | | | | | |
|---|---|---|---|---|---|---|---|---|
| | | (Activity; | | | | | Ratio: IC50 | Ratio: IC50 |
| Conjugate | modified | +5 pM | IC50 | | IC50 | | toxicity/IC50 | hemolysis/IC50 |
| Sample | group | EGFdianthin) | (Toxicity) | | (hemolysis) | | activity | activity |
| SO1861 | none | 500 nM | 9000 | nM | 10.000 | nM | 18 | 20 |
| SO1861- | Aldehyde | 4000 nM | >100.000 | nM | >1.000.000 | nM | >25 | >250 |
| EMCH | | | | | | | | |
| SO1861- | Aldehyde | 4000 nM | >100.000 | nM | >1.000.000 | nM | >25 | >250 |
| NHS | | | | | | | | |

TABLE 4
IC50 overview of modified SO1861 activity (A431 cells)/toxicity (A431 cells)/hemolysis
(red blood cells)/CMC, for indicated SO1861 derivatives and SO1861.

| | | IC50 nM | | | | | | |
|---|---|---|---|---|---|---|---|---|
| | | (Activity; +5 | | | | | Ratio: IC50 | Ratio: IC50 |
| Conjugate | modified | pM | IC50 | | IC50 | | toxicity/IC50 | hemolysis/IC50 |
| Sample | group | EGFdianthin) | (Toxicity) | | (hemolysis) | | activity | activity |
| SO1861 | none | 300 nM | 2000 | nM | 10.000 | nM | 7 | 33 |
| SO1861- | Aldehyde | 3000 nM | >30.000 | nM | >1.000.000 | nM | >10 | >333 |
| EMCH | | | | | | | | |
| SO1861- | Aldehyde | 3000 nM | >30.000 | nM | >1.000.000 | nM | >10 | >333 |
| NHS | | | | | | | | |

1-42. (canceled)

43. A saponin derivative based on a saponin comprising a triterpene aglycone core structure and at least one of a first saccharide chain ‘R¹’ and a second saccharide chain ‘R²’ linked to the aglycone core structure,
wherein the saponin derivative comprises an aglycone core structure comprising a hydrazone functional group according to formula (A) 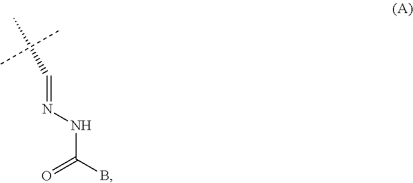
wherein B is a LINKER A,
or
a hydrazone functional group according to formula (I) 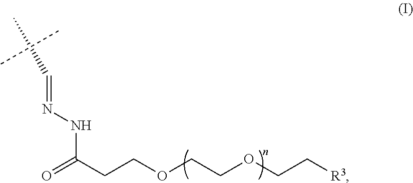
wherein n is an integer selected from 0-15,
R³ is azide, OH, or a linker selected from (IV)a-j: 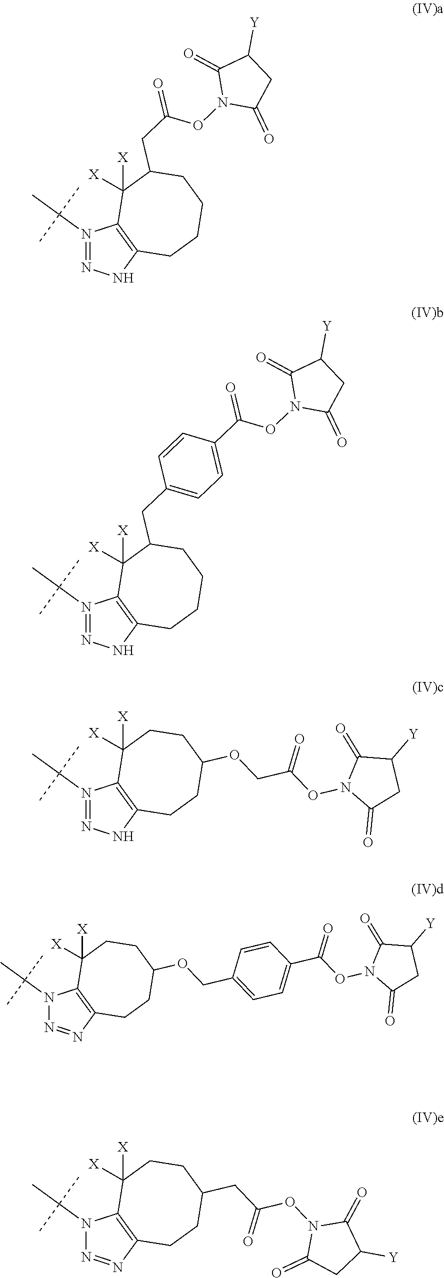 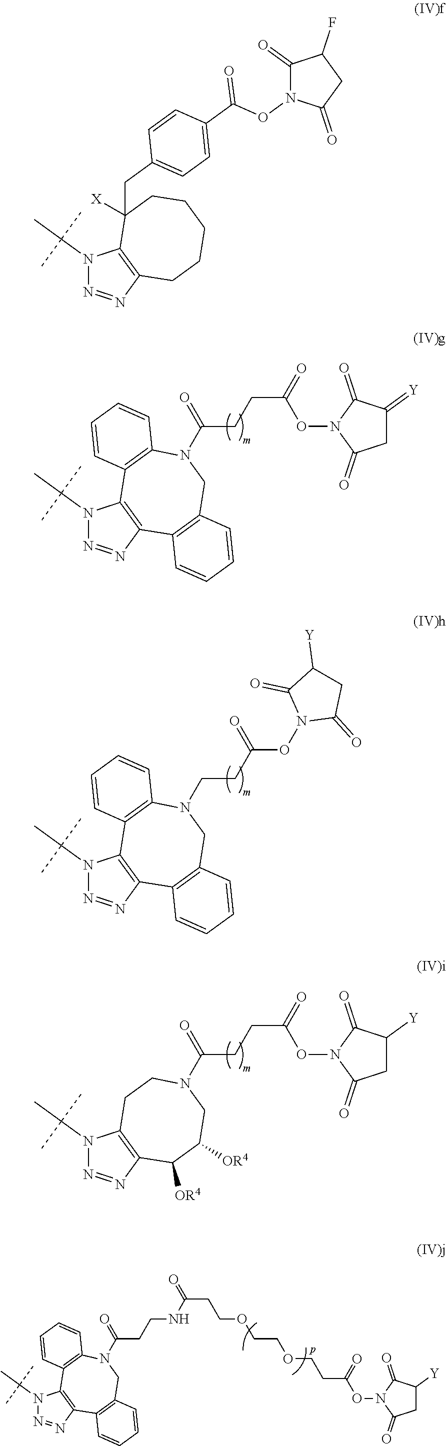
wherein
X is H or F;
Y is H or SO₂ONa;
m is 1 or 2;
p is an integer selected from 0-5 and
R⁴ is a C1-C3 alkyl chain.

44. A saponin derivative according to claim 43, wherein the saponin on which the saponin derivative is based is a mono-desmosidic or bi-desmosidic triterpene saponin belonging to the type of a 12,13-dehydrooleanane with the aldehyde group in position C-23 and optionally comprising a glucuronic acid group in a carbohydrate substituent at the C-3beta-OH group of the saponin.

45. A saponin derivative according to claim 43, wherein n is an integer selected from 1-12.

46. A saponin derivative according to claim 43, wherein
R¹ is selected from:
H,
GlcA-,
Glc-,
Gal-,
Rha-(1→2)-Ara-,
Gal-(1→2)-[Xyl-(1→3)]-GlcA-,
Glc-(1→2)-[Glc-(1→4)]-GlcA-,
Glc-(1→2)-Ara-(1→3)-[Gal-(1→2)]-GlcA-,
Xyl-(1→2)-Ara-(1→3)-[Gal-(1→2)]-GlcA-,
Glc-(1→3)-Gal-(1→2)-[Xyl-(1→3)]-Glc-(1→4)-Gal-,
Rha-(1→2)-Gal-(1→3)-[Glc-(1→2)]-GlcA-,
Ara-(1→4)-Rha-(1→2)-Glc-(1→2)-Rha-(1→2)-GlcA-,
Ara-(1→4)-Fuc-(1→2)-Glc-(1→2)-Rha-(1→2)-GlcA-,
Ara-(1→4)-Rha-(1→2)-Gal-(1→2)-Rha-(1→2)-GlcA-,
Ara-(1→4)-Fuc-(1→2)-Gal-(1→2)-Rha-(1→2)-GlcA-,
Ara-(1→4)-Rha-(1→2)-Glc-(1→2)-Fuc-(1→2)-GlcA-,
Ara-(1→4)-Fuc-(1→2)-Glc-(1→2)-Fuc-(1→2)-GlcA-,
Ara-(1→4)-Rha-(1→2)-Gal-(1→2)-Fuc-(1→2)-GlcA-,
Ara-(1→4)-Fuc-(1→2)-Gal-(1→2)-Fuc-(1→2)-GlcA-,
Xyl-(1→4)-Rha-(1→2)-Glc-(1→2)-Rha-(1→2)-GlcA-,
Xyl-(1→4)-Fuc-(1→2)-Glc-(1→2)-Rha-(1→2)-GlcA-,
Xyl-(1→4)-Rha-(1→2)-Gal-(1→2)-Rha-(1→2)-GlcA-,
Xyl-(1→4)-Fuc-(1→2)-Gal-(1→2)-Rha-(1→2)-GlcA-,
Xyl-(1→4)-Rha-(1→2)-Glc-(1→2)-Fuc-(1→2)-GlcA-,
Xyl-(1→4)-Fuc-(1→2)-Glc-(1→2)-Fuc-(1→2)-GlcA-,
Xyl-(1→4)-Rha-(1→2)-Gal-(1→2)-Fuc-(1→2)-GlcA-,
Xyl-(1→4)-Fuc-(1→2)-Gal-(1→2)-Fuc-(1→2)-GlcA-, and
derivatives thereof, and/or
R² is selected from:
H,
Glc-,
Gal-,
Rha-(1→2)-[Xyl-(1→4)]-Rha-,
Rha-(1→2)-[Ara-(1→3)-Xyl-(1→4)]-Rha-,
Ara-,
Xyl-,
Xyl-(1→4)-Rha-(1→2)-[R¹-(→4)]-Fuc- wherein R¹ is 4E-Methoxycinnamic acid,
Xyl-(1→4)-Rha-(1→2)-[R²-(→4)]-Fuc- wherein R² is 4Z-Methoxycinnamic acid,
Xyl-(1→4)- [Gal-(1→3)]-Rha-(1→2)-4-OAc-Fuc-,
Xyl-(1→4)- [Glc-(1→3)]-Rha-(1→2)-3,4-di-OAc-Fuc-,
Xyl-(1→4)- [Glc-(1→3)]-Rha-(1→2)-[R⁵-(→4)]-3-OAc-Fuc- wherein R⁵ is 4E-Methoxycinnamic acid,
Glc-(1→3)-Xyl-(1→4)- [Glc-(1→3)]-Rha-(1→2)-4-OAc-Fuc-,
Glc-(1→3)-Xyl-(1→4)-Rha-(1→2)-4-OAc-Fuc-,
(Ara- or Xyl-)(1→3)-(Ara- or Xyl-)(1→4)-(Rha- or Fuc-)(1→2)[4-OAc-(Rha- or Fuc-)(1→4)]-(Rha- or Fuc-),
Xyl-(1→3)-Xyl-(1→4)-Rha-(1→2)-[Qui-(1→4)]-Fuc-,
Api-(1→3)-Xyl-(1→4)-[Glc-(1→3)]-Rha-(1→2)-Fuc-,
Xyl-(1→4)-[Gal-(1→3)]-Rha-(1→2)-Fuc-,
Xyl-(1→4)-[Glc-(1→3)]-Rha-(1→2)-Fuc-,
Ara/Xyl-(1→4)-Rha/Fuc-(1→4)-[Glc/Gal-(1→2)]-Fuc-,
Api-(1→3)-Xyl-(1→4)-[Glc-(1→3)]-Rha-(1→2)-[R⁶-(→4)]-Fuc- wherein R⁶ is 5-O-[5-O-Ara/Api-3,5-dihydroxy-6-methyl-octanoyl]-3,5-dihydroxy-6-methyl-octanoic acid,
Api-(1→3)-Xyl-(1→4)-Rha-(1→2)-[R⁷-(→4)]-Fuc- wherein R⁷ is 5-O-[5-O-Ara/Api-3,5-dihydroxy-6-methyl-octanoyl]-3,5-dihydroxy-6-methyl-octanoic acid,
Api-(1→3)-Xyl-(1→4)-Rha-(1→2)-[Rha-(1→3)]-4-OAc-Fuc-,
Api-(1→3)-Xyl-(1→4)-[Glc-(1→3)]-Rha-(1→2)-[Rha-(1→3)]-4-OAc-Fuc-,
6-OAc-Glc-(1→3)-Xyl-(1→4)-Rha-(1→2)-[3-OAc-Rha-(1→3)]-Fuc-,
Glc-(1→3)-Xyl-(1→4)-Rha-(1→2)-[3-OAc-Rha-(1→3)]-Fuc-,
Xyl-(1→3)-Xyl-(1→4)-Rha-(1→2)-[Qui-(1→4)]-Fuc-,
Glc-(1→3)-[Xyl-(1→4)]-Rha-(1→2)-[Qui-(1→4)]-Fuc-,
Glc-(1→3)-Xyl-(1→4)-Rha-(1→2)-[Xyl-(1→3)-4-OAc-Qui-(1→4)]-Fuc-,
Xyl-(1→3)-Xyl-(1→4)-Rha-(1→2)-[3,4-di-OAc-Qui-(1→4)]-Fuc-,
Glc-(1→3)-[Xyl-(1→4)]-Rha-(1→2)-Fuc-,
6-OAc-Glc-(1→3)-[Xyl-(1→4)]-Rha-(1→2)-Fuc-,
Glc-(1→3)-[Xyl-(1→3)-Xyl-(1→4)]-Rha-(1→2)-Fuc-,
Xyl-(1→3)-Xyl-(1→4)-Rha-(1→2)-[Xyl-(1→3)-4-OAc-Qui-(1→4)]-Fuc-,
Api/Xyl-(1→3)-Xyl-(1→4)-[Glc-(1→3)]-Rha-(1→2)-[Rha-(1→3)]-40Ac-Fuc-,
Api-(1→3)-Xyl-(1→4)-[Glc-(1→3)]-Rha-(1→2)-[Rha-(1→3)]-40Ac-Fuc-,
Api/Xyl-(1→3)-Xyl-(1→4)-[Glc-(1→3)]-Rha-(1→2)-[R⁸-(→4)]-Fuc- wherein R⁸ is 5-O-[5-O-Rha-(1→2)-Ara/Api-3,5-dihydroxy-6-methyl-octanoyl]-3,5-dihydroxy-6-methyl-octanoic acid,
Api/Xyl-(1→3)-Xyl-(1→4)-[Glc-(1→3)]-Rha-(1→2)-[R⁹-(→4)]-Fuc- wherein R⁹ is 5-O-[5-O-Ara/Api-3,5-dihydroxy-6-methyl-octanoyl]-3,5-dihydroxy-6-methyl-octanoic acid,
Api/Xyl-(1→3)-Xyl-(1→4)-[Glc-(1→3)]-Rha-(1→2)-[R¹⁰-(→4)]-Fuc- wherein R¹⁰ is 5-O-[5-O-Ara/Api-3,5-dihydroxy-6-methyl-octanoyl]-3,5-dihydroxy-6-methyl-octanoic acid,
Api-(1→3)-Xyl-(1→4)-Rha-(1→2)-[R¹¹-(→4)]-Fuc- wherein R¹¹ is 5-O-[5-O-Ara/Api-3,5-dihydroxy-6-methyl-octanoyl]-3,5-dihydroxy-6-methyl-octanoic acid,
Xyl-(1→3)-Xyl-(1→4)-Rha-(1→2)-[R¹²-(→4)]-Fuc- wherein R¹² is 5-O-[5-O-Ara/Api-3,5-dihydroxy-6-methyl-octanoyl]-3,5-dihydroxy-6-methyl-octanoic acid,
Api-(1→3)-Xyl-(1→4)-Rha-(1→2)-[R¹³-(→3)]-Fuc- wherein R¹³ is 5-O-[5-O-Ara/Api-3,5-dihydroxy-6-methyl-octanoyl]-3,5-dihydroxy-6-methyl-octanoic acid,
Xyl-(1→3)-Xyl-(1→4)-Rha-(1→2)-[R¹⁴-(→3)]-Fuc- wherein R¹⁴ is 5-O-[5-O-Ara/Api-3,5-dihydroxy-6-methyl-octanoyl]-3,5-dihydroxy-6-methyl-octanoic acid
Glc-(1→3)-[Glc-(1→6)]-Gal-, and
derivatives thereof.

47. A saponin derivative according to claim 43, wherein
R¹ is selected from:
Gal-(1→2)-[Xyl-(1→3)]-GlcA-; and/or
R² is selected from:
Glc-(1→3)-Xyl-(1→4)-Rha-(1→2)-[Xyl-(1→3)-4-OAc-Qui-(1→4)]-Fuc-,
Xyl-(1→3)-Xyl-(1→4)-Rha-(1→2)-[3,4-di-OAc-Qui-(1→4)]-Fuc-,
Api-(1→3)-Xyl-(1→4)-Rha-(1→2)-[R¹¹-(→4)]-Fuc- wherein R¹¹ is 5-O-[5-O-Ara/Api-3,5-dihydroxy-6-methyl-octanoyl]-3,5-dihydroxy-6-methyl-octanoic acid,
Xyl-(1→3)-Xyl-(1→4)-Rha-(1→2)-[R¹²-(→4)]-Fuc- wherein R¹² is 5-O-[5-O-Ara/Api-3,5-dihydroxy-6-methyl-octanoyl]-3,5-dihydroxy-6-methyl-octanoic acid,
Api-(1→3)-Xyl-(1→4)-Rha-(1→2)-[R¹³-(→3)]-Fuc- wherein R¹³ is 5-O-[5-O-Ara/Api-3,5-dihydroxy-6-methyl-octanoyl]-3,5-dihydroxy-6-methyl-octanoic acid, and
Xyl-(1→3)-Xyl-(1→4)-Rha-(1→2)-[R¹⁴-(→3)]-Fuc- wherein R¹⁴ is 5-O-[5-O-Ara/Api-3,5-dihydroxy-6-methyl-octanoyl]-3,5-dihydroxy-6-methyl-octanoic acid,
preferably R¹ is Gal-(1→2)-[Xyl-(1→3)]-GlcA- and R² is Glc-(1→3)-Xyl-(1→4)-Rha-(1→2)-[Xyl-(1→3)-4-OAc-Qui-(1→4)]-Fuc-.

48. A saponin derivative according to claim 43, wherein the saponin derivative is a derivative of a saponin selected from the group of saponins consisting of: Quillaja bark saponin, NP-017777, NP-017778, NP-017774, NP-018110, NP-017772, NP-018109, NP-017888, NP-017889, NP-018108, SA1641, AE X55, NP-017674, NP-017810, AG1, NP-003881, NP-017676, NP-017677, NP-017706, NP-017705, NP-017773, NP-017775, SA1657, AG2, 501861, GE1741, 501542, 501584, 501658, 501674, 501832, 501904, 501862, QS-7, QS1861, QS-7 api, QS1862, QS-17, QS-18, QS-21 A-apio, QS-21 A-xylo, QS-21 B-apio, QS-21 B-xylo and Agrostemmoside E (AG1856).

49. A saponin derivative according to claim 43, wherein Y=H.

50. A saponin derivative according to claim 43, wherein the linker is selected from the group consisting of linkers (IV)g-(IV)j.

51. A saponin derivative according to claim 43, wherein m=1.

52. A saponin derivative according to claim 43, wherein the saponin derivative is a compound according to formula (VIII)

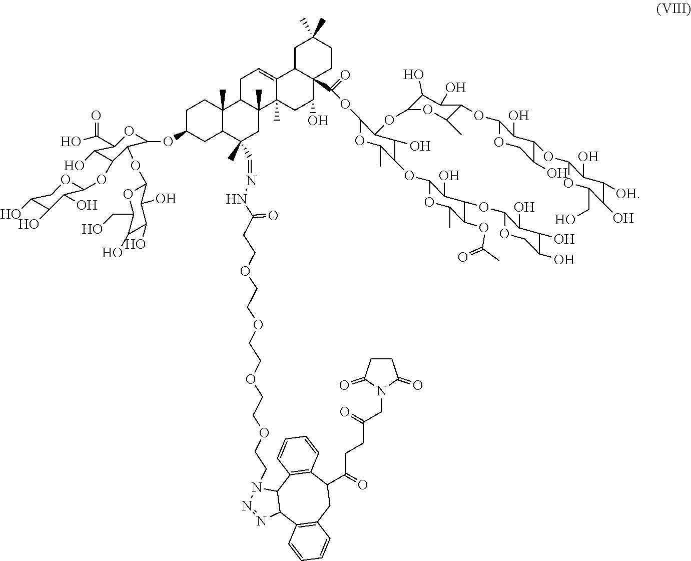

53. A pharmaceutical composition comprising a saponin derivative according to claim 43.

54. A kit comprising:
(a) the pharmaceutical composition of claim 53; and
(b) a second pharmaceutical composition comprising any one or more of: a conjugate of a cell-surface molecule binding-molecule and an effector moiety, an antibody-effector moiety conjugate, a receptor-ligand—effector moiety conjugate, an antibody-toxin conjugate, a receptor-ligand—toxin conjugate, an antibody-drug conjugate, a receptor-ligand—drug conjugate, an antibody-oligonucleotide conjugate or a receptor-ligand—oligonucleotide conjugate.

55. The pharmaceutical composition of claim 53, further comprising a conjugate of a cell-surface molecule binding-molecule and an effector moiety, a receptor-ligand—effector moiety conjugate, an antibody-toxin conjugate, a receptor-ligand—toxin conjugate, an antibody-drug conjugate, a receptor-ligand—drug conjugate, an antibody-nucleic acid conjugate or a receptor-ligand—nucleic acid conjugate.

56. An in vitro, ex vivo, or in vivo method for transferring a molecule from outside a cell to inside said cell comprising:
a) providing a cell;
b) providing the molecule for transferring from outside the cell into the cell provided in step a);
c) providing a saponin derivative according to claim 43;
d) contacting the cell of step a) in vitro or ex vivo with the molecule of step b) and the saponin derivative of step c), therewith establishing the transfer of the molecule from outside the cell into said cell.

57. A saponin conjugate based on a saponin derivative according to claim 43, wherein the N-hydroxy succinimide active ester functional group 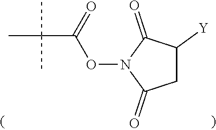
is transformed to an amide functional group (—C(O)—N(H)—) through reaction with a second proteinaceous molecule (‘proteinaceous molecule 2’) comprising an amine functional group according to formula (XI)

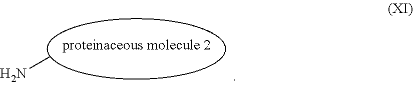

58. A pharmaceutical combination comprising:
(a) a pharmaceutical composition comprising the saponin conjugate of claim 57; and
(b) a pharmaceutical composition comprising a conjugate comprising a third proteinaceous molecule (‘proteinaceous molecule 3’), and an effector moiety, wherein the proteinaceous molecule 3 is the same or different from the proteinaceous molecule 2 present in the saponin conjugate, the proteinaceous molecule 3 comprising a third binding site for binding to a third epitope of a third cell-surface molecule, wherein the third cell-surface molecule, if different from the first cell surface molecule and/or the second cell surface molecule, is present on the same cell as the first cell surface.

59. A pharmaceutical combination comprising:
(a) a pharmaceutical composition comprising the saponin conjugate of claim 57 which comprises the first binding site for binding to the first epitope on the first cell-surface molecule; and
(b) a pharmaceutical composition comprising a conjugate comprising a fourth proteinaceous molecule (‘proteinaceous molecule 4’) and an effector moiety, wherein the proteinaceous molecule 4 comprises the first binding site for binding to the first epitope on the cell-surface molecule of (a),
wherein the first binding site of the proteinaceous molecule 2 and the first binding site of the proteinaceous molecule 4 are the same, and wherein the first cell-surface molecule and the first epitope on the first cell-surface molecule, to which the proteinaceous molecule 2 can bind, and the first cell-surface molecule and the first epitope on the first cell-surface molecule, to which the proteinaceous molecule 4 can bind, are the same.

60. A method of treating cancer or rheumatoid arthritis in a patient in need thereof comprising administering an effective dose of the pharmaceutical combination of claim 58 to the patient.

61. A method of treating cancer or rheumatoid arthritis in a patient in need thereof comprising administering an effective dose of the pharmaceutical combination of claim 59 to the patient.

62. A process for preparing a saponin derivative comprising reacting a saponin derivative of claim 43 with a compound with a free amine group.
